(19) United States
(12) Patent Application Publication
(10) Pub. No.: US 2026/0132218 A1
(43) Pub. Date: May 14, 2026

(54) FULLY HUMAN MONOCLONAL ANTIBODIES AGAINST HUMAN HER-2

(71) Applicant: A & G Pharmaceutical, Inc., Columbia, MD (US)

(72) Inventor: Ginette Serrero, Ellicott City, MD (US)

(21) Appl. No.: 19/120,405

(22) PCT Filed: Oct. 13, 2023
(86) PCT No.: PCT/US2023/076877

§ 371 Date: Apr. 11, 2025

Related U.S. Application Data
(60) Provisional application No. 63/416,270, filed on Oct. 14, 2022.

(51) Int. Cl. C07K 16/32; A61P 35/00

(52) U.S. Cl. CPC C07K 16/32; A61P 35/00; C07K 2317/55; C07K 2317/565; C07K 2317/73; C07K 2317/77; C07K 2317/92

(57) ABSTRACT

Described herein are antibodies, particularly monoclonal antibodies, that specifically bind to human HER2 and are useful for the treatment of cancer in patients.

### RELATED APPLICATIONS

This application claims priority to U.S. Ser. No. 63/416,270 filed on Oct. 14, 2022, which is incorporated herein by reference in its entirety.

### FIELD OF THE DISCLOSURE

This disclosure relates to antibodies, including fully human monoclonal antibodies (mAbs), that specifically bind to and internalize human epidermal growth factor-2, also known as HER-2, as well as methods of manufacturing (e.g., cell lines) and using the antibodies (e.g., treating cancer).

### BACKGROUND OF THE DISCLOSURE

The human epidermal growth factor receptor (HER) family of receptors plays a central role in the pathogenesis of several human cancers. They are involved in cellular proliferation and differentiation by acting through multiple signaling pathways. The superfamily is composed of four major members HER1 (also known as Epidermal Growth Factor Receptor EGFR), HER2, HER3 and HER4 also call erbB1, erbB2, ErbB3 and ErbB4. All the members are characterized by a cysteine-rich extracellular domain including the ligand binding site, a transmembrane lipophilic region and an intracellular domain with a tyrosine kinase enzymatic activity. The HER receptors exist as monomers on the cell surface. Upon ligand binding, the receptors homo or heterodimerize with other HER members leading to phosphorylation. HER2 is a 125 amino-acid, 185 kDa transmembrane glycoprotein located on chromosome 17q12. It is overexpressed in many tissues to facilitate excessive/uncontrolled growth and tumorigenesis. HER2 has no known direct activating ligand and maybe constitutively activated or become active upon heterodimerization with other HER members leading to autophosphorylation of tyrosine residues within the intracellular domain triggering a variety of signaling pathways namely MAP-K, PI3K resulting in cellular proliferation, survival, migration, and angiogenesis.

HER2 is overexpressed in several cancers. Most studies have been carried out with breast cancers after it was found that HER2 induced mammary carcinogenesis in vitro and in vivo. Overexpression with or without gene amplification occurs in 15-30% of breast cancers and this has both prognostic and predictive implications. HER2 overexpression and amplification is associated with shorter disease-free and overall survivals and with resistance to certain hormonal agents as well as increased risk of metastasis to the brain.

In addition to being overexpressed in breast cancers, HER2 overexpression has been reported in gastric cancer, esophageal cancer, ovarian cancer and endometrial cancer. In lung cancer, overexpression as well as HER2 mutation has been reported in 30% of adenocarcinomas. Similar observation has been reported with bladder carcinomas. However clinical trials of HER2 therapy for lung and bladder cancers have had limited success so far.

Two categories of treatment targeting HER2 and its biological activity have been developed, FDA approved and are used in the standard of care: monoclonal antibodies raised against HER2 and small molecule tyrosine kinase inhibitors targeting HER2 and EGFR such as lapatinib and neratinib and targeting all members of the HER family such as Afatinib.

Trastuzumab also named Herceptin is a humanized anti-HER2 monoclonal antibody that binds to domain IV of the extracellular segment of the HER2, blocking its signaling. Its mechanisms of action include inhibition of HER2 dependent cell signaling, inhibition of PI3K-AKT pathway, inhibition of angiogenesis and antibody-dependent cellular toxicity. Trastuzumab was approved along with a companion diagnostic for breast cancer patients overexpressing Her2 as 3+ by immunohistochemistry (IHC) or positive by Fluorescence In Situ Hybridization (FISH). It is administered in combination therapy with standard of care chemotherapeutic agents. Trastuzumab was also approved as combination therapy for treatment of HER2 overexpressing metastatic gastric or gastroesophageal junction adenocarcinoma.

Pertuzumab is a humanized anti-HER2 monoclonal antibody that blocks the activation of HER2 receptor by inhibiting its dimerization. It elicits action at a different ligand binding site from trastuzumab. It is approved for in combination with trastuzumab and docetaxel in Her2 positive metastatic breast cancer patients.

The antibody drug conjugate Ado-trastuzumab Emtansine was developed based on the fact that anti-HER2 antibody trastuzumab is internalizing and that as such it can deliver a cytotoxic payload to the cells leading to cell killing. Ado-Trastuzumab-Emtansine (Kadcyla) consists of trastuzumab conjugated to the drug mertansine DM1 (“TDM1”). TDM1 is approved as a single agent for treatment of HER2-positive metastatic breast cancer patients who have already received trastuzumab and a taxane.

The antibody drug conjugate (fam-trastuzumab-deruxtecan-nhki (Enhertu)) is another antibody drug conjugate using Trastuzumab to deliver a topoisomerase inhibitor deruxtecan. It is approved for unresectable or metastatic HER2-positive breast cancer who have received a prior anti-HER2-based regimen and for locally advanced or metastatic HER2-positive gastric or gastroesophageal junction adenocarcinoma who have received a prior trastuzumab-based regimen.

The most developed anti-HER2 targeted therapies are based on trastuzumab and pertuzumab antibodies. Both of these antibodies are derived from mouse monoclonal antibodies that have been humanized by molecular biology recombinant methods. Thus, there is an art-recognized opportunity to develop a new class of anti-Her2 antibody, particularly fully human anti-HER2 monoclonal antibodies that can be developed by immunizing fully human mice. Such anti-HER2 antibodies, as well as methods for making and using the same are provided by this disclosure.

### SUMMARY OF THE DISCLOSURE

Provided herein are monoclonal antibodies (mAbs), including humanized anti-HER2 antibodies, as well as antigen binding fragments such as polypeptides including complementarity determining regions (CDRs) which bind HER-2. In some embodiments, this disclosure describes herein is an isolated antibody, or antigen binding fragment thereof, that binds human HER2 and further competes for binding with antibodies 11D7, 2A2, 12E3, 2D7, 3G7, 8C1, 2A1, 7F9, 9E4, 11C9, or 12A6; the heavy and light chain variable regions thereof; and/or a polypeptide comprising the CDRs of such antibodies (see Tables 1 and 2); and/or derivatives thereof (e.g., comprising conservative amino acid substitutions thereto (see, e.g., Table 4)), any of which would be considered an equivalent thereof. In particular, the mAbs herein include the anti-human HER2 antibodies having the amino acid sequences for the respective CDRs recited in Table 1. In some embodiments, this disclosure provides fully human anti-HER2 antibodies that are internalized into cells expressing HER2 and compete with trastuzumab for binding to HER2, as well as methods for using such antibodies for their neutralizing as well as internalizing properties. In preferred embodiments, such antibodies include those referred to herein as 11D7, 2A2, 12E3, 2D7, 3G7, 8C1, and/or the CDRs comprising by such antibodies (see Table 1); the heavy and light chain variable regions thereof (see Table 3); and/or derivatives thereof (e.g., comprising conservative amino acid substitutions thereto (see, e.g., Table 4)). In some embodiments, this disclosure provides fully human anti-HER2 antibodies that are internalized and do not compete with trastuzumab for binding to HER2 receptor, as well as methods for studying their internalizing properties and biological properties by examining their ability to be cytotoxic (e.g., kill cells) as antibody drug conjugates. In preferred embodiments, such antibodies include those referred to herein as 2A1, 7F9, 9E4, 11C9, 12A6; comprising the heavy and light chain variable regions thereof (see Table 3); and/or comprising the CDRs of Table 2; and/or derivatives thereof (e.g., comprising conservative amino acid substitutions thereto (see, e.g., Table 4)). The antibodies can further include the complete heavy and/or light chain of the antibodies shown in Table 3, and/or a derivative thereof, that include the CDRs shown in Tables 1 and 2. In some preferred embodiments, such antibodies can have the amino acid sequences of the variable heavy (“VH”) or variable light (“VL”) polypeptides (VH or VL “chains”, respectively) shown below for the antibody or equivalent thereof shown in Table 3. In some embodiments, the isolated antibodies, or antigen binding fragments, described herein, bind human HER2 with a sufficient K_{D}, such as from about 10⁻⁸ molar (M) to about 10⁻¹⁰ molar (M), preferably up to about 3.6×10⁻⁹ M, or any value in between that provides sufficient (e.g., detectable) binding to human HER2, as determined using any suitable method used in field, such as but not limited to the scouting assay by Biolayer-Interferometry using an Octet Red 96 machine (see, e.g., the Examples section herein), or as measured by any assay available to the skilled artisan.

In some embodiments, this disclosure provides nucleotide sequences encoding a 11D7, 2A2, 12E3, 2D7, 3G7, 8C1, 2A1, 7F9, 9E4, 11C9, or 12A6 antibody (or functional fragment thereof such as a CDR and/or variable region), and/or a particular amino acid sequence of an equivalent to an, wherein equivalents may be easily derived from the amino acid sequence of any of SEQ ID NOS. 62-83 and the information presented in Table 4, as well as using the functional and other assays disclosed herein, and/or as may be otherwise available to those of ordinary skill in the art. In some embodiments, this disclosure also provides expression vectors including an isolated nucleic acid comprising and/or consisting of such nucleotide sequences (in preferred embodiments any of SEQ ID NOS. 62-83 or derivatives thereof) as well as host cells (e.g., a cell line) containing such an expression vector.

In some embodiments, this disclosure provides methods for using the antibodies and/or fragments thereof (e.g., CDRs) (which are referred to collectively as “antibodies” herein unless otherwise indicated) for treating cancers involving HER2. In some embodiments, this disclosure also provides methods of treating cancer in a patient by identifying a patient having cancerous cells expressing HER2 and administering to the patient an antibody or antigen binding fragment as described herein. A variety of human cancers are known to express HER2, including ovarian, breast, multiple myelomas, lung, renal carcinoma, prostate, hepatocellular carcinoma, uterine, bladder, biliary, esophageal, gastric, laryngeal, brain, leukemia and glioblastoma.

Unless defined otherwise, all technical and scientific terms used herein have the same meaning as commonly understood by those of ordinary skill in the art. It is to be understood that both the foregoing general description and the following detailed description are exemplary and explanatory only and are not restrictive. Additional features will be set forth in part in the description which follows or may be learned by practice as described herein. The foregoing and other features will become apparent to one skilled in the art upon consideration of the following description of exemplary embodiments.

### BRIEF DESCRIPTION OF THE DRAWINGS

The accompanying drawings and photographs, which are incorporated in and constitute a part of this specification, illustrate several embodiments and together with the description serve to explain the principles herein.

FIG. 1 illustrates the blocking activity of antibodies 11D7, 8C1, 2A2, 2D7, 12E3, and 3G7 with respect to trastuzumab for binding to HER2.

FIG. 2. Antibodies 12E3 and 12A6 were each separately conjugated with DM1 and the two resulting ADCs 12E3-DM1 and 12A6-DM1 were examined for their ability to inhibit proliferation of HER2 overexpressing breast cancer cells in vitro alone or in combination on two HER2 overexpressing cell line AU565.

FIG. 3. Antibodies 12E3 and 12A6 were each separately conjugated with DM1 and the two resulting ADCs 12E3-DM1 and 12A6-DM1 were examined for their ability to inhibit proliferation of HER2 overexpressing breast cancer cells in vitro alone or in combination on two HER2 overexpressing cell line SKBR3.

### DETAILED DESCRIPTION

This disclosure relates to antibodies, including but not limited to fully human monoclonal antibodies, that specifically bind to human HER2 and which can be used to treat cancer, such as breast cancer. In some embodiments, this disclosure provides an isolated antibody, one of more antigen binding fragment(s) thereof, that binds human HER2 and further competes for binding with antibodies 11D7, 2A2, 12E3, 2D7, 3G7, 8C1, 2A1, 7F9, 9E4, 11C9, or 12A6; the heavy and light chain variable regions thereof (see Table 3); and/or a polypeptide comprising the CDRs of such antibodies (see Tables 1 and 2); and/or derivatives thereof (e.g., comprising conservative amino acid substitutions thereto (see, e.g., Table 4)), any of which would be considered an equivalent thereof. In particular, the mAbs herein include the anti-human HER2 antibodies having the amino acid sequences for the respective CDRs recited in Table 1. In some embodiments, this disclosure provides fully human anti-HER2 antibodies that are internalized into cells expressing HER2 and compete with trastuzumab for binding to HER2, as well as methods for using such antibodies for their neutralizing as well as internalizing properties. In preferred embodiments, such antibodies include those referred to herein as 11D7, 2A2, 12E3, 2D7, 3G7, 8C1, and/or the CDRs comprising by such antibodies (see Table 1); the heavy and light chain variable regions thereof (see Table 3); and/or derivatives thereof (e.g., comprising conservative amino acid substitutions thereto (see, e.g., Table 4)). In some embodiments, this disclosure provides fully human anti-her2 antibodies that are internalized and do not compete with trastuzumab for binding to HER2 receptor, as well as methods for studying their internalizing properties. In preferred embodiments, such antibodies include those referred to herein as 2A1, 7F9, 9E4, 11C9, 12A6; comprising the heavy and light chain variable regions thereof (see Table 3); and/or comprising the CDRs of Table 2; and/or derivatives thereof (e.g., comprising conservative amino acid substitutions thereto (see, e.g., Table 4)). The antibodies can further include at least a portion of (most preferably including the CDRs thereof) and/or the complete heavy and/or light chain of the antibodies shown in Table 3, and/or a derivative thereof, that include the CDRs shown in Tables 1 and 2. In some preferred embodiments, such antibodies can have the amino acid sequences of the variable heavy (“VH”) or variable light (“VL”) polypeptides (VH or VL “chains”, respectively) shown below for the antibody or equivalent thereof shown in Table 3. In some embodiments, the isolated antibodies, or antigen binding fragments, described herein, bind human HER2 with a K_{D} of up to 3.6×10⁻⁹ molar (M), as measured by Octet assay described below (see, e.g., the Examples section herein), or as measured by any assay available to the skilled artisan. In some embodiments, this disclosure provides nucleotide sequences encoding a particular amino acid sequence of an equivalent to an 11D7, 2A2, 12E3, 2D7, 3G7, 8C1, 2A1, 7F9, 9E4, 11C9, or 12A6 antibody may be easily derived from the amino acid sequence of any of SEQ ID NOS. 62-83 and the information presented in Table 4, and/or equivalents/derivatives thereof. In some embodiments, this disclosure also provides expression vectors including an isolated nucleic acid comprising and/or consisting of such nucleotide sequences (in preferred embodiments any of SEQ ID NOS. 62-83 or derivatives thereof) as well as host cells (e.g., a cell line) containing such an expression vector. In some embodiments, this disclosure provides methods for using the antibodies and/or fragments thereof (e.g., CDRs) (which are referred to collectively as “antibodies” herein unless otherwise indicated) for treating cancers involving HER2. In some embodiments, this disclosure also provides methods of treating cancer in a patient by identifying a patient having cancerous cells expressing HER2 and administering to the patient an antibody or antigen binding fragment as described herein. A variety of human cancers are known to express HER2, including ovarian, breast, multiple myelomas, lung, renal carcinoma, prostate, hepatocellular carcinoma, uterine, bladder, biliary, esophageal, gastric, laryngeal, brain, leukemia and glioblastoma. The use of combinations of antibodies, such as one or more described herein with another available to those of ordinary skill in the art, are also contemplated herein. For instance, in some embodiments, the combinations may be identified to provide statistically significant differences from results (e.g., neutralization assays) obtained using only one or more of the antibodies and not others. In some embodiments, combinations exhibit additive and/or, preferably synergistic, activity. In some embodiments, the combination may comprise a an 11D7, 2A2, 12E3, 2D7, 3G7, 8C1, 2A1, 7F9, 9E4, 11C9, or 12A6 antibody (or derivative thereof) and trastuzumab, pertuzumab, the antibody drug conjugate Ado-trastuzumab Emtansine (Kadcyla consisting of trastuzumab conjugated to the drug mertansine (DM1) (TDM1)), and/or the antibody drug conjugate (fam-trastuzumab-deruxtecan-nhki (Enhertu). Mertansine (DM1; N₂′-deacetyl-N₂′-(3-mercapto-1-oxopropyl)-maytansine) is shown below:

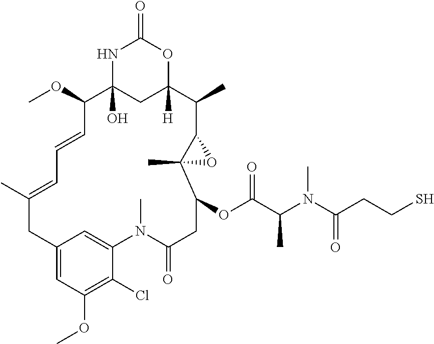

DM1 can be conjugated to a antibody (“mab” in the graphic below) as shown below using the linker 4-mercaptovaleric acid as shown below:

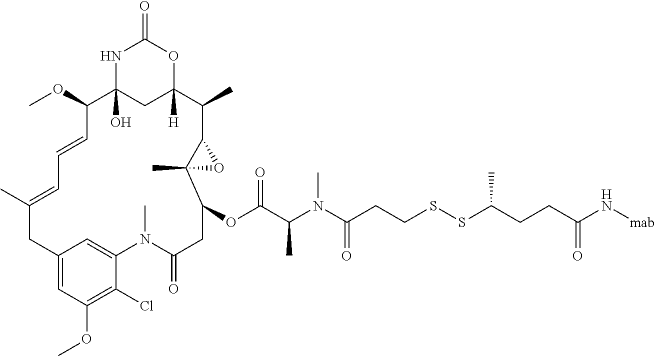

DM1 can also be linked to an antibody using 4-(3-mercapto-2,5-dioxo-1-pyrrolidinylmethyl)-cylohexanecarboxylic acid (SMCC), which is indicated by the term emtansine, as shown below:

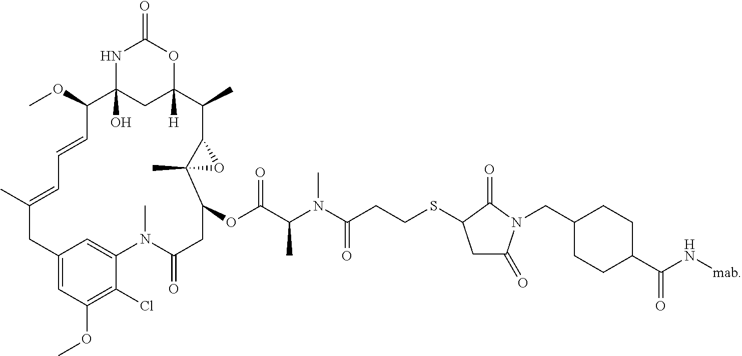

The antibodies of this disclosure can also be combined with chemotherapeutic agents used in the standard of care some of which are used in combination with anti-HER2 therapies. The antibodies of such compositions may be different entities such as two or more different monoclonal antibodies or derivatives thereof or may be found on the same entity such as a bi-functional antibody (a single antibody or derivative thereof comprising multiple binding specificities). Such combinations as described herein may also be combined with one or more other agents that may affect immune cell function such as antibodies against CTLA-4, and the like. One of ordinary skill in the art would recognize that many such combinations may be suitable for use as described herein.

The term “antibody” as used herein means a whole antibody and any antigen binding fragment (i.e., “antigen-binding portion”) or single chain thereof. A whole human antibody is a glycoprotein comprising at least two heavy (H) chains and two light (L) chains inter-connected by di-sulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “isolated antibody” refers to an antibody and/or fragment thereof that is substantially free of other antibodies and/or fragments thereof having different antigenic specificities (e.g., an isolated antibody that specifically binds HER2, is substantially free of antibodies that specifically bind antigens other than HER2). An isolated antibody that specifically binds HER2 may, however, have cross-reactivity to other antigens, e.g., HER2 from species other than human. Moreover, an isolated antibody may be substantially free of other cellular material and/or chemicals. The term “antigen binding portion” or “antigen binding fragment” of an antibody, as used herein, refers to one or more fragments of an intact antibody that retain the ability to specifically bind to a given antigen (e.g., human granulin). Antigen binding functions of an antibody can be performed by fragments of an intact antibody. Examples of binding fragments encompassed within the term antigen binding portion or antigen binding fragment of an antibody include a Fab fragment, a monovalent fragment consisting of the V_{L}, V_{H}, CL and CH1 domains; a F(ab)₂ fragment, a bivalent fragment comprising two Fab fragments linked by a disulfide bridge at the hinge region; an Fd fragment consisting of the V_{H} and CH1 domains; an Fv fragment consisting of the V_{L} and V_{H} domains of a single arm of an antibody; a single domain antibody (dAb) fragment (Ward et al., 1989 Nature 341:544-546), which consists of a V_{H} domain or a V_{L} domain; and an isolated complementarity determining region (CDR). Furthermore, although the two domains of the Fv fragment, V_{L} and V_{H}, are coded for by separate genes, they can be joined, using recombinant methods, by an artificial peptide linker that enables them to be made as a single protein chain in which the V_{L} and V_{H} regions pair to form monovalent molecules (known as single chain Fv (scFv); see, e.g., Bird et al., 1988 Science 242:423-426; and Huston et al., 1988 Proc. Natl. Acad. Sci. 85:5879-5883). Such single chain antibodies include one or more antigen binding portions or fragments of an antibody. These antibody fragments are obtained using conventional techniques known to those of skill in the art, and the fragments are screened for utility in the same manner as are intact antibodies. Antigen binding fragments can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology, 23, 9, 1126-1136). Antigen binding portions of antibodies can be grafted into scaffolds based on polypeptides such as Fibronectin type III (Fn3) (see U.S. Pat. No. 6,703,199, which describes fibronectin polypeptide monobodies). Antigen binding fragments can be incorporated into single chain molecules comprising a pair of tandem Fv segments (V_{H}-CH1-V_{H}-CH1) which, together with complementary light chain polypeptides, form a pair of antigen binding regions (Zapata et al., 1995 Protein Eng. 8 (10): 1057-1062; and U.S. Pat. No. 5,641,870). The term “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. For example, a mouse antibody can be modified by replacing its constant region with the constant region from a human immunoglobulin. Due to the replacement with a human constant region, the chimeric antibody can retain its specificity in recognizing the antigen while having reduced antigenicity in human as compared to the original mouse antibody. The terms “monoclonal antibody” or “monoclonal antibody composition” as used herein refer to a preparation of antibody molecules of single molecular composition. A monoclonal antibody composition displays a single binding specificity and affinity for a particular epitope. The term “human antibody”, as used herein, is intended to include antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences. The human antibodies may include amino acid residues not encoded by human sequences (e.g., mutations introduced by random or site-specific mutagenesis in vitro or by somatic mutation in vivo). The term “human monoclonal antibody” refers to antibodies displaying a single binding specificity which have variable regions in which both the framework and CDR regions are derived from human sequences. In one embodiment, the human monoclonal antibodies are produced by hybridomas which include (i) a B cell obtained from a transgenic non-human animal, e.g., a transgenic mouse, having a genome comprising a human heavy chain transgene and a light chain transgene (ii) fused to an immortalized cell. A “humanized” antibody is an antibody that retains the reactivity of a non-human antibody while being less immunogenic in humans. This can be achieved, for instance, by retaining the non-human CDR regions and replacing the remaining parts of the antibody with their human counterparts (i.e., the constant region as well as the framework portions of the variable region). See, e.g., Morrison et al., Proc. Natl. Acad. Sci. USA, 81:6851-6855, 1984; Morrison and Oi, Adv. Immunol., 44:65-92, 1988; Verhoeyen et al., Science, 239:1534-1536, 1988; Padlan, Molec. Immun., 28:489-498, 1991; and Padlan, Molec. Immun., 31:169-217, 1994. Other examples of human engineering technology include, but are not limited to, Xoma technology disclosed in U.S. Pat. No. 5,766,886.

The term “isotype” refers to the antibody class (e.g., IgM, IgE, IgG such as IgG1 or IgG4) that is provided by the heavy chain constant region genes. Isotype also includes modified versions of one of these classes, where modifications have been made to alter the Fc function, for example, to enhance or reduce effector functions or binding to Fc receptors. Isotype also refers to the antibody class (e.g., kappa, lambda) that is provided by the light-chain constant regions. The antibody may contain an Fc region including one or more mutations that influence one or more antibody properties, such as stability, pattern of glycosylation or other modifications, effector cell function, pharmacokinetics, and so forth. In some embodiments, an antibody has reduced or minimal glycosylation. In some embodiments, an antibody has ablated or reduced effector function. Exemplary Fc mutations include without limitation (i) a human IgG1 Fc region mutations L234A, L235A, G237A, and N297A; (ii) a human IgG2 Fc region mutations A330S, P331S and N297A; and (iii) a human IgG4 Fc region mutations S228P, E233P, F234V, L235A, delG236, and N297A (EU numbering). In some embodiments, the human IgG2 Fc region comprises A330S and P331S mutations. In some embodiments, the human IgG4 Fc region comprises an S288P mutation. In some embodiments, the human IgG4 Fc region comprises S288P and L235E mutations. Antibodies that target cell surface antigens can trigger immunostimulatory and effector functions that are associated with Fc receptor (FcR) engagement on immune cells. There are a number of Fc receptors that are specific for particular classes of antibodies, including IgG (gamma receptors), IgE (eta receptors), IgA (alpha receptors) and IgM (mu receptors). Binding of the Fc region to Fc receptors on cell surfaces can trigger a number of biological responses including phagocytosis of antibody-coated particles (antibody-dependent cell-mediated phagocytosis, or ADCP), clearance of immune complexes, lysis of antibody-coated cells by killer cells (antibody-dependent cell-mediated cytotoxicity, or ADCC) and release of inflammatory mediators, placental transfer, and control of immunoglobulin production. Additionally, binding of the C1 component of complement to antibodies can activate the complement system. Activation of complement can be important for the lysis of cellular pathogens. However, the activation of complement can also stimulate the inflammatory response and can also be involved in autoimmune hypersensitivity or other immunological disorders. Variant Fc regions with reduced or ablated ability to bind certain Fc receptors are useful for developing therapeutic antibodies and Fc-fusion polypeptide constructs which act by targeting, activating, or neutralizing ligand functions while not damaging or destroying local cells or tissues. An Fc domain monomer refers to a polypeptide chain that includes second and third antibody constant domains (e.g., CH2 and CH3). In some embodiments, an Fc domain monomer also includes a hinge domain. In some embodiments, the Fc domain monomer is of any immunoglobulin antibody isotype, including IgG, IgE, IgM, IgA, and IgD. Additionally, in some embodiments, an Fc domain monomer is of any IgG subtype (e.g., IgG1, IgG2, IgG2a, IgG2b, IgG2c, IgG3, and IgG4). Additional mutations in the Fc domain and the biological consequences of those mutations are well known in the art and can be applied to the antibodies herein. See, e.g., US Patent Application Publication No. 20220002434.

The term “binding specificity” as used herein refers to the ability of an individual antibody combining site to react with only one antigenic determinant (e.g., an epitope). The term “epitope” means a protein determinant capable of specific binding to an antibody. Epitopes usually consist of chemically active surface groupings of molecules such as amino acids or sugar side chains and usually have specific three-dimensional structural characteristics, as well as specific charge characteristics. Conformational and non-conformational epitopes are distinguished in that the binding to the former but not the latter is lost in the presence of denaturing solvents. The phrase “specifically (or selectively) binds” to an antibody (e.g., an human HER2-binding antibody) refers to a binding reaction that is determinative of the presence of a cognate antigen in a heterogeneous population of proteins and other biologics. The phrases “an antibody recognizing an antigen” and “an antibody specific for an antigen” are used interchangeably herein with the term “an antibody which binds specifically to an antigen”.

The antibodies can be used as a treatment for cancer (e.g., breast cancer, including advanced metastatic breast cancer or other diseases which exhibit an increased expression of HER2. By the term “neutralizing” it shall be understood that the antibody has the ability to inhibit or block any biological activity of HER2 that leads to tumorigenesis, including its ability to stimulate cell proliferation, survival or to induce tumor growth in experimental animals and in humans. An effective amount of anti-HER2 antibody is administered to a mammal, including humans, by various routes.

As used herein, the term “affinity” refers to the strength of interaction between antibody and antigen at single antigenic sites. Within each antigenic site, the variable region of the antibody “arm” interacts through weak non-covalent forces with antigen at numerous sites (e.g., epitopes); wherein the higher the number of interactions between the antibody and the epitope, the stronger the affinity between the two. The term “K_{assoc}” or “Ka”, as used herein, is intended to refer to the association rate of a particular antibody-antigen interaction, whereas the term “Kdis” or “Kd,” as used herein, is intended to refer to the dissociation rate of a particular antibody-antigen interaction. The term “K_{D}”, as used herein, is intended to refer to the dissociation constant, which is obtained from the ratio of Kd to Ka (i.e. Kd/Ka) and is expressed as a molar concentration (M). K_{D} values for antibodies can be determined using methods well established in the art. Methods for determining the K_{D} of an antibody include measuring surface plasmon resonance using a biosensor system such as a Biacore system or measuring affinity in solution-by-solution equilibrium titration (SET) or Octet Red 96 by Biolayer Interferometry. As used herein, the term “high affinity” for an antibody or antigen binding fragment thereof (e.g., a Fab fragment) generally refers to an antibody, or antigen binding fragment, having a K_{D} of 10⁻⁹ M or less.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, Y-carboxyglutamate, and O-phosphoserine. Amino acid analogs refer to compounds that have the same basic chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “conservatively modified variant” applies to both amino acid and nucleic acid sequences (e.g., see Tables 4 and 5). With respect to particular nucleic acid sequences, conservatively modified variants refer to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid that encodes a polypeptide is implicit in each described sequence.

The terms “identical” or 100% percent “identity,” in the context of two or more nucleic acids or polypeptide sequences, refer to two or more sequences or subsequences that are the same. Two sequences are “substantially identical” if two sequences have a specified percentage of amino acid residues or nucleotides that are the same (i.e., 60% identity, optionally 65%, 70%, 75%, 80%, 85%, 90%, 95%, or 99% identity over a specified region, or, when not specified, over the entire sequence), when compared and aligned for maximum correspondence over a comparison window, or designated region as measured using one of the following sequence comparison algorithms or by manual alignment and visual inspection. Optionally, the identity exists over a region that is at least about 50 nucleotides (or 10 amino acids) in length, or over a region that is 100 to 500 or 1000 or more nucleotides (or 20, 50, 200 or more amino acids) in length. For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch, J. Mol. Biol. 48:443, 1970, by the search for similarity method of Pearson and Lipman, Proc. Nat'l. Acad. Sci. USA 85:2444, 1988, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Brent et al., Current Protocols in Molecular Biology, John Wiley & Sons, Inc. (Ringbou ed., 2003)). Two examples of algorithms that are suitable for determining percent sequence identity and sequence similarity are the BLAST and BLAST 2.0 algorithms, which are described in Altschul et al., Nuc. Acids Res. 25:3389-3402, 1977; and Altschul et al., J. Mol. Biol. 215:403-410, 1990, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=4 and a comparison of both strands. For amino acid sequences, the BLASTP program uses as defaults a wordlength of 3, and expectation (E) of 10, and the BLOSUM62 scoring matrix (see Henikoff and Henikoff, Proc. Natl. Acad. Sci. USA 89:10915, 1989) alignments (B) of 50, expectation (E) of 10, M=5, N=−4, and a comparison of both strands. The BLAST algorithm also performs a statistical analysis of the similarity between two sequences (see, e.g., Karlin and Altschul, Proc. Natl. Acad. Sci. USA 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, or less than about 0.01, or less than about 0.001. The percent identity between two amino acid sequences can also be determined using the algorithm of E. Meyers and W. Miller (Comput. Appl. Biosci., 4:11-17, 1988) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol, Biol. 48:444-453, 1970) algorithm which has been incorporated into the GAP program in the GCG software package (available on the world wide web at gcg.com), using either a Blossom 62 matrix or a PAM250 matrix, and a gap weight of 16, 14, 12, 10, 8, 6, or 4 and a length weight of 1, 2, 3, 4, 5, or 6.

Other than percentage of sequence identity noted above, another indication that two nucleic acid sequences or polypeptides are substantially identical is that the polypeptide encoded by the first nucleic acid is immunologically cross reactive (e.g., blocking) with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be achieved by generating sequences in which the third position of one or more selected (or all) codons is substituted with mixed-base and/or deoxyinosine residues (Batzer et al., Nucleic Acid Res. 19:5081, 1991; Ohtsuka et al., J. Biol. Chem. 260:2605-2608, 1985; and Rossolini et al., Mol. Cell. Probes 8:91-98, 1994).

The term “operably linked” refers to a functional relationship between two or more polynucleotide (e.g., DNA) segments. Typically, the term refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system. Generally, promoter transcriptional regulatory sequences that are operably linked to a transcribed sequence are physically contiguous to the transcribed sequence, i.e., they are cis-acting. However, some transcriptional regulatory sequences, such as enhancers, need not be physically contiguous or located in close proximity to the coding sequences whose transcription they enhance.

As used herein, the term, “optimized” means that a nucleotide sequence has been altered to encode an amino acid sequence using codons that are preferred in the production cell or organism, generally a eukaryotic cell, for example, a cell of Pichia, a Chinese Hamster Ovary cell (CHO) or a human cell. The optimized nucleotide sequence is engineered to retain completely or as much as possible the amino acid sequence originally encoded by the starting nucleotide sequence, which is also known as the “parental” sequence. The optimized sequences herein have been engineered to have codons that are preferred in mammalian cells. However, optimized expression of these sequences in other eukaryotic cells or prokaryotic cells is also envisioned herein. The amino acid sequences encoded by optimized nucleotide sequences are also referred to as optimized.

The terms “polypeptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. Unless otherwise indicated, a particular polypeptide sequence also implicitly encompasses conservatively modified variants thereof.

The term “recombinant human antibody”, as used herein, includes all human antibodies that are prepared, expressed, created or isolated by recombinant means, such as antibodies isolated from an animal (e.g., a mouse) that is transgenic or transchromosomal for human immunoglobulin genes or a hybridoma prepared therefrom, antibodies isolated from a host cell transformed to express the human antibody, e.g., from a transfectoma, antibodies isolated from a recombinant, combinatorial human antibody library, and antibodies prepared, expressed, created or isolated by any other means that involve splicing of all or a portion of a human immunoglobulin gene, sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “recombinant host cell” (or simply “host cell”) or “cell line” refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” or “cell line” as used herein. A cultured cell comprising the vector is also provided. In some embodiments, the cultured cell may be a cultured cell transfected with the vector or a progeny of the cell, wherein the cell expresses the immunogenic polypeptide. Suitable cell lines are known to those of skill in the art and are commercially available, for example, through the American Type Culture Collection (ATCC). The transfected cells can be used in a method of producing an antibody. The method comprises culturing a cell comprising the vector under conditions that allow expression of the antibody, optionally under the control of an expression sequence. The immunogenic polypeptide can be isolated from the cell or the culture medium using standard protein purification methods.

The term “subject” includes human and non-human animals. Non-human animals include all vertebrates (e.g.: mammals and non-mammals) such as, non-human primates (e.g.: cynomolgus monkey), rodent, sheep, dog, cow, chicken, llama (no light chain), alpaca (no light chain), camel (no light chain), shark (no light chain), amphibians, and reptiles. Except when noted, the terms “patient” or “subject” are used herein interchangeably.

As used herein, the term “treating” or “treatment” of any disease or disorder (e.g., breast cancer) refers in one embodiment, to ameliorating the disease or disorder (i.e., slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to preventing or delaying the onset or development or progression of the disease or disorder. “Prevention” as it relates to indications described herein, including, conditions or disorders associated with cancers that express HER2.

The term “vector” is intended to refer to a polynucleotide molecule capable of transporting another polynucleotide to which it has been linked. One type of vector is a “plasmid”, which refers to a circular double stranded DNA loop into which additional DNA segments may be ligated. Another type of vector is a viral vector, such as an adeno-associated viral vector (AAV, or AAV2), wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, it is intended to include such other forms of expression vectors, such as viral vectors (e.g., replication defective retroviruses, adenoviruses and adeno-associated viruses), which serve equivalent functions. Various viral vectors that have been successfully utilized for introducing a nucleic acid to a host include retrovirus, adenovirus, adeno-associated virus (AAV), herpes virus, and poxvirus, among others. The vectors may be constructed using standard recombinant techniques widely available to one skilled in the art. Such techniques may be found in common molecular biology references such as Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), and PCR Protocols: A Guide to Methods and Applications (Innis, et al. 1990. Academic Press, San Diego, ca). “Non-viral” plasmid vectors may also be suitable in certain embodiments. Preferred plasmid vectors are compatible with bacterial, insect, and/or mammalian host cells. Such vectors include, for example, PCR-ii, PCR3, and pcDNA3.1 (Invitrogen, San Diego, CA), pBSii (Stratagene, La Jolla, CA), pet15 (Novagen, Madison, WI), pGEX (Pharmacia Biotech, Piscataway, NJ), pEGFp-n2 (Clontech, Palo Alto, CA), pETI (Bluebacii, Invitrogen), pDSR-alpha (PCT pub. No. WO 90/14363) and pFASTBACdual (Gibco-BRL, Grand island, NY) as well as Bluescript® plasmid derivatives (a high copy number COLe1-based phagemid, Stratagene Cloning Systems, La Jolla, CA), PCR cloning plasmids designed for cloning TAQ-amplified PCR products (e.g., TOPO™ TA Cloning® kit, PCR2.1 plasmid derivatives, Invitrogen, Carlsbad, CA). Bacterial vectors may also be used. These vectors include, for example, Shigella, Salmonella, Vibrio cholerae, Lactobacillus, Bacille Calmette Guérin (BCG), and Streptococcus (see for example, WO 88/6626; WO 90/0594; WO 91/13157; WO 92/1796; and WO 92/21376). Many other non-viral plasmid expression vectors and systems are known in the art and may be use. Other delivery techniques may also suffice including, for example, DNA-ligand complexes, adenovirus-ligand-DNA complexes, direct injection of DNA, CaPO₄ precipitation, gene gun techniques, electroporation, and colloidal dispersion systems. Colloidal dispersion systems include macromolecule complexes, nanocapsules, microspheres, beads, and lipid-based systems including oil-in-water emulsions, micelles, mixed micelles, and liposomes. The preferred colloidal system is a liposome, which are artificial membrane vesicles useful as delivery vehicles in vitro and in vivo. RNA, DNA and intact virions can be encapsulated within the aqueous interior and be delivered to cells in a biologically active form (Fraley, R., et al., 1981, Trends Biochem. Sci., 6:77). The composition of the liposome is usually a combination of phospholipids, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Particularly useful are diacylphosphatidylglycerols, where the lipid moiety contains from 14-18 carbon atoms, particularly from 16-18 carbon atoms, and is saturated. Illustrative phospholipids include egg phosphatidylcholine, dipalmitoylphosphatidylcholine and distearoylphosphatidylcholine.

Antibodies can be conjugated with drugs to form antibody-drug conjugates (ADCs). Typically, the ADC contains a linker between the drug and the antibody. The linker can be a degradable or a non-degradable linker, cleavable or non-cleavable linker. Degradable linkers are typically easily degraded in the intracellular environment, for example, the linker is degraded at the target site, so that the drug is released from the antibody. Suitable degradable linkers include, for example, enzymatically degraded linkers, including peptidyl-containing linkers that can be degraded by intracellular proteases (such as lysosomal proteases or endosomal proteases), or sugar linkers, for example, a glucuronide-containing linker that can be degraded by glucuronidase. The peptidyl linker may include, for example, dipeptides such as valine-citrulline, phenylalanine-lysine or valine-alanine. Other suitable degradable linkers include, for example, pH-sensitive linkers (for example, linkers that are hydrolyzed at a pH of less than 5.5, such as hydrazone linkers) and linkers that degrade under reducing conditions (for example, disulfide bond linkers). Non-degradable linkers typically release the drug under conditions where the antibody is hydrolyzed by a protease.

Before being connected to the antibody, the linker has a reactive group capable of reacting with certain amino acid residues, and the connection is achieved through the reactive group. Sulfhydryl-specific reactive groups are preferred and include, for example, maleimide compounds, halogenated amides (such as iodine, bromine, or chloro); halogenated esters (such as iodine, bromine, or chloro); halogenated methyl ketones (such as iodine, bromine or chloro), benzyl halides (such as iodine, bromine or chloro); vinyl sulfone, pyridyl disulfide; mercury derivatives such as 3,6-Di-(mercury methyl) dioxane, and the counter ion is acetate, chloride or nitrate; and polymethylene dimethyl sulfide thiosulfonate. The linker may include, for example, maleimide linked to the antibody via thiosuccinimide.

The drug can be any cytotoxic, inhibiting cell growth or immunosuppressive drug. In embodiments, the linker connects the antibody and the drug, and the drug has a functional group that can be bonded to the linker. For example, the drug may have an amino group, a carboxyl group, a sulfhydryl group, a hydroxyl group, or a ketone group that can form a bond with the linker. In the case where the drug is directly connected to the linker, the drug has a reactive active group before being connected to the antibody. The antibodies disclosed herein and/or derivatives thereof may also be adjoined to and/or conjugated to functional agents for in vitro and/or in vivo use. Useful drug categories include, for example, anti-tubulin drugs, DNA minor groove binding reagents, DNA replication inhibitors, alkylating reagents, antibiotics, folate antagonists, antimetabolites, chemotherapy sensitizers, topoisomerase inhibitors, Vinca Alkaloids, etc. Typical cytotoxic drugs include, for example, auristatins, camptothecins, duocarmycins, etoposides, maytansines and maytansinoids (e.g., DM1 and DM4), taxanes, benzodiazepines or benzodiazepine containing drugs (e.g., pyrrolo[1,4]benzodiazepines (PBDs), indolinobenzodiazepines and oxazolidinobenzodiazepines and vinca alkaloids. In some embodiments, the antibodies could be adjoined to and/or conjugated to functional moieties such as cytotoxic drugs or toxins, and/or active fragments thereof such as diphtheria A chain, exotoxin A chain, ricin A chain, abrin A chain, curcin, crotin, phenomycin, enomycin, among others. Suitable functional moieties may also include radiochemicals. Antibodies may be adjoined to and/or conjugated to the one or more functional agents using standard techniques in the art.

As described herein, the drug-linker can be used to form ADC in one simple step. In other embodiments, bifunctional linker compounds can be used to form ADCs in a two-step or multi-step process. For example, the cysteine residue reacts with the reactive part of the linker in the first step, and in the subsequent step, the functional group on the linker reacts with the drug to form ADC. Many suitable methods for creating a drug-linker used to form an ADC are known to those of ordinary skill in the art and would be suitable for use with the reagents disclosed herein (e.g., the antibodies and derivatives thereof) as well as others (e.g., cytotoxic agents not explicitly listed herein) available to such persons.

Generally, the functional group on the linker is selected to facilitate the specific reaction with the appropriate reactive group on the drug moiety. As a non-limiting example, the azide-based moiety can be used to specifically react with the reactive alkynyl group on the drug moiety. The drug is covalently bound to the linker through the 1,3-dipolar cycloaddition between the azide and alkynyl groups. Other useful functional groups include, for example, ketones and aldehydes (suitable for reacting with hydrazides and alkoxyamines), phosphines (suitable for reacting with azides); isocyanates and isothiocyanates (suitable for reaction with amines and alcohols); and activated esters, such as N-hydroxysuccinimide ester (suitable for reaction with amines and alcohols). These and other ligation strategies, such as those described in “Bioconjugation Technology”, Second Edition (Elsevier), are well known to those skilled in the art. Those skilled in the art can understand that for the selective reaction between the drug moiety and the linker, when a complementary pair of reactive functional groups is selected, each member of the complementary pair can be used for both linkers and drugs.

Provided herein are monoclonal antibodies, including humanized anti-HER2 antibodies, as well as antigen binding fragments such as polypeptides including complementarity determining regions (CDRs) which bind HER-2. In some embodiments, this disclosure provides fully human anti-HER2 antibodies that are internalized into cells expressing HER2 and compete with trastuzumab for binding to HER2, as well as methods for using such antibodies for their neutralizing as well as internalizing properties. In some embodiments, this disclosure provides fully human anti-HER2 antibodies that are internalized and do not compete with trastuzumab for binding to HER2 receptor, as well as methods for studying their internalizing and cell growth inhibiting properties. In some embodiments, this disclosure provides methods for using the antibodies and/or fragments thereof (e.g., CDRs) (which are referred to collectively as “antibodies” herein unless otherwise indicated) for treating cancers involving HER2.

The skilled artisan has many suitable techniques for using the antibodies (e.g., antibodies) described herein to identify biological samples containing proteins that bind thereto. For instance, antibodies may be utilized to isolate HER2 or cells expressing HER2 and/or expressing HIV antigens using, for example, immunoprecipitation or other capture-type assay. This well-known technique is performed by attaching the antibody to a solid support or chromatographic material (e.g., a bead coated with Protein A, Protein G and/or Protein L). The bound antibody is then introduced into a solution either containing or believed to contain HER2 antigens (e.g., an HER2-expressing cell). The HER2 antigen(s) may then bind to the antibody and non-binding materials are washed away under conditions in which the HIV antigen(s) remains bound to the antibody. The bound protein may then be separated from the antibody and analyzed as desired. Similar methods for isolating a protein using an antibody are well-known in the art. The antibodies (e.g., antibodies) may also be utilized to detect HIV or HIV antigens within a biological sample. For instance, the antibodies may be used in assays such as, for example, flow cytometric analysis, ELISA, immunoblotting (e.g., western blot), in situ detection, immunocytochemistry, and/or immunohistochemistry. Methods of carrying out such assays are well-known in the art. In some embodiments, the antibodies may be adjoined to and/or conjugated to one or more detectable labels. For instance, suitable detectable labels may include, for instance, fluorosceins (e.g., DyLight, Cy3, Cy5, FITC, HiLyte Fluor 555, HiLyte Fluor 647; 5-carboxy-2,7-dichlorofluorescein; 5-Carboxyfluorescein (5-FAM); 5-HAT (Hydroxy Tryptamine); 5-Hydroxy Tryptamine (HAT); 6-JOE; 6-carboxyfluorescein (6-FAM); FITC; 6-carboxy-1,4-dichloro-2′,7′-dichlorofluorescein (TET); 6-carboxy-1,4-dichloro-2′,4′, 5′, 7′-tetrachlorofluorescein (HEX); 6-carboxy-4′,5′-dichloro-2′, 7′-dimethoxyfluorescein (JOE); Alexa fluors (e.g., 350, 405, 430, 488, 500, 514, 532, 546, 555, 568, 594, 610, 633, 635, 647, 660, 680, 700, 750); BODIPY fluorophores (e.g., 492/515, 493/503, 500/510, 505/515, 530/550, 542/563, 558/568, 564/570, 576/589, 581/591, 630/650-X, 650/665-X, 665/676, FL, FL ATP, FI-Ceramide, R6G SE, TMR, TMR-X conjugate, TMR-X, SE, TR, TR ATP, TR-X SE)), rhodamines (e.g., 110, 123, B, B 200, BB, BG, B extra, 5-carboxytetramethylrhodamine (5-TAMRA), 5 GLD, 6-Carboxyrhodamine 6G, Lissamine, Lissamine Rhodamine B, Phallicidine, Phalloidine, Red, Rhod-2, ROX (6-carboxy-X-rhodamine), 5-ROX (carboxy-X-rhodamine), Sulphorhodamine B can C, Sulphorhodamine G Extra, TAMRA (6-carboxytetramethylrhodamine), Tetramethylrhodamine (TRITC), WT), Texas Red, and/or Texas Red-X. Other detectable labels known in the art may also be suitable for use. Antibodies, such as antibodies, may be adjoined to and/or conjugated to the one or more detectable labels using standard techniques in the art.

The antibodies described herein may also be used to determine the presence of a disease state in a patient, to predict prognosis, or to determine the effectiveness of a chemotherapeutic or other treatment regimen. Expression profile assays, performed as described herein or as is otherwise known in the art, may be used to determine the relative level of expression of HER2 in a cell, for instance. The level of expression may then be correlated with base (e.g., control) levels to determine whether a particular disease is present within the patient, the patient's prognosis, or whether a particular treatment regimen is effective. For example, if the patient is being treated with a particular anti-infective regimen, an increased or decreased level of expression of HER2 in the patient's tissues (e.g., in plasma) may indicate the regimen is worsening or improving the load of HER2-expressing cells (e.g., cancer) in that host. The increase or decrease in expression may indicate the regimen is having or not having the desired effect and another therapeutic modality may therefore be selected.

It is also possible to use the antibodies described herein as reagents in drug screening assays to test, for example, new drug candidates. The reagents may be used to ascertain the effect of a drug candidate on the expression of the immunogenic target in a cell line, or a cell or tissue of a patient. The expression profiling technique may be combined with high throughput screening techniques to allow rapid identification of useful compounds and monitor the effectiveness of treatment with a drug candidate (see, for example, Zlokarnik, et al., Science 279, 84-8 (1998)). Drug candidates may be chemical compounds, nucleic acids, proteins, antibodies, or derivatives therefrom, whether naturally occurring or synthetically derived. Drug candidates thus identified may be utilized, among other uses, as pharmaceutical compositions for administration to patients or for use in further screening assays.

The antibodies (e.g., polypeptides) and nucleic acids described herein may also be combined with one or more pharmaceutically acceptable carriers prior to administration to a host. A pharmaceutically acceptable carrier is a material that is not biologically or otherwise undesirable, e.g., the material may be administered to a subject, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art. Suitable pharmaceutical carriers and their formulations are described in, for example, Remington's: The Science and Practice of Pharmacy, 21^{st} Edition, David B. Troy, ed., Lippicott Williams & Wilkins (2005). Typically, an appropriate amount of a pharmaceutically-acceptable salt is used in the formulation to render the formulation isotonic. Examples of the pharmaceutically-acceptable carriers include, but are not limited to, sterile water, saline, buffered solutions like Ringer's solution, and dextrose solution. The pH of the solution is generally from about 5 to about 8 or from about 7 to about 7.5. Other carriers include sustained-release preparations such as semipermeable matrices of solid hydrophobic polymers containing polypeptides or fragments thereof. Matrices may be in the form of shaped articles, e.g., films, liposomes or microparticles. It will be apparent to those persons skilled in the art that certain carriers may be more preferable depending upon, for instance, the route of administration and concentration of composition being administered. Carriers are those suitable for administration of polypeptides and/or fragments thereof to humans or other subjects. Pharmaceutical compositions may also include carriers, thickeners, diluents, buffers, preservatives, surface active agents, adjuvants, immunostimulants, in addition to the immunogenic polypeptide. Pharmaceutical compositions may also include one or more active ingredients such as antimicrobial agents, anti-inflammatory agents and anesthetics. The pharmaceutical composition may be administered orally, parentally, by inhalation spray, rectally, intranodally, or topically in dosage unit formulations containing conventional pharmaceutically acceptable carriers, adjuvants, and vehicles. The term “pharmaceutically acceptable carrier” or “physiologically acceptable carrier” as used herein refers to one or more formulation materials suitable for accomplishing or enhancing the delivery of a nucleic acid, polypeptide, or peptide as a pharmaceutical composition. A “pharmaceutical composition” is a composition comprising a therapeutically effective amount of a nucleic acid or polypeptide. The terms “effective amount” and “therapeutically effective amount” each refer to the amount of an antibody, nucleic acid or the like used to observe the desired therapeutic effect (e.g., eliminating HER2-expressing cells, e.g., cancerous HER2-expressing cells).

Methods for treating one or more disease conditions (e.g., cancer) in a mammalian host comprising administering to the mammal at least one or more effective doses of one or more antibodies (and/or derivative(s) thereof) described herein are also provided. In some embodiments, the antibody is a monoclonal antibody or fragment or derivative thereof comprising one or more of SEQ ID NOS. 1-61; the amino acid sequences shown in Tables 1 and 2 and/or encoded by a nucleotide sequence of Table 3; and/or substituted derivatives and/or fragments thereof; as well as in some embodiments conservatively substituted variants thereof. The one or more antibodies may be administered in a dosage amount of about 1 to about 50 mg/kg, about 1 to about 30 mg/kg, or about 5 to about 30 mg/kg (e.g., about any of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, or 40 mg/kg). In certain embodiments, the one or more antibodies may be administered to the mammal (e.g., intradermally, intravenously, orally, rectally) at about 10 mg/kg one or more times. When multiple doses are administered, the doses may comprise about the same or different amount of antibody in each dose. The doses may also be separated in time from one another by the same or different intervals. For instance, the doses may be separated by about any of 6, 12, 24, 36, 48, 60, 72, 84, or 96 hours, one week, two weeks, three weeks, one month, two months, three months, four months, five months, six months, seven months, eight months, nine months, 10 months, 11 months, 12 months, 1.5 years, 2 years, 3 years, 4 years, 5 years, or any time period before, after, and/or between any of these time periods. In some embodiments, the antibodies may be administered in conjunction with other agents (e.g., anti-infective agents and/or chemotherapeutic agent). Such other agents may be administered about simultaneously with the antibodies, or at a different time and/or frequency. Other embodiments of such methods may also be appropriate as could be readily determined by one of ordinary skill in the art.

To assist the skilled artisan in using the antibodies such as antibodies described herein, the same may be provided in kit format. A kit including one or more of such antibodies and optionally other components necessary for using the same to detect cells expressing HIV is also provided. The antibodies of the kit may be provided in any suitable form, including frozen, lyophilized, or in a pharmaceutically acceptable buffer such as TBS or PBS. The kit may also include other reagents required for utilization of the antibodies in vitro or in vivo such as buffers (e.g., TBS, PBS), blocking agents (solutions including nonfat dry milk, normal sera, Tween-20 Detergent, BSA, or casein), and/or detection reagents (e.g., goat anti-mouse IgG biotin, streptavidin-HRP conjugates, allophycocyanin, B-phycoerythrin, R-phycoerythrin, peroxidase, detectable labels, and other labels and/or staining kits (e.g., ABC Staining Kit, Pierce)). The kits may also include other reagents and/or instructions for using the antibodies in commonly utilized assays described above such as, for example, flow cytometric analysis, ELISA, immunoblotting (e.g., western blot), in situ detection, immunocytochemistry, and/or immunohistochemistry. In one embodiment, the kit provides an antibody in purified form. In another embodiment, the antibody may be provided in biotinylated form either alone or along with an avidin-conjugated detection reagent (e.g., antibody). In another embodiment, the kit includes an antibody comprising one or more detectable labels that may be used to directly detect HER2. Buffers and the like required for using any of these systems are well-known in the art and/or may be prepared by the end-user or provided as a component of the kit. The kit may also include a solid support containing positive- and negative-control protein and/or tissue samples. For example, kits for performing spotting or western blot-type assays may include control cell or tissue lysates for use in SDS-PAGE or nylon or other membranes containing pre-fixed control samples with additional space for experimental samples. Kits for visualization of HIV in cells on slides may include pre-formatted slides containing control cell or tissue samples with additional space for experimental samples. Other embodiments of kits are also contemplated herein as would be understood by those of ordinary skill in the art.

The isolated antibodies, or antigen binding fragments, described herein, bind human HER2 with a K_{D} of up to 3.6×10⁻⁹ molar (M)). This is within the range of Kd measured for commercially available trastuzumab which is having a Kd of 3.1×10⁻¹⁰M in our experiments and the reported manufactured trastuzumab reported to have a Kd of 5×10⁻⁹M as measured by Octet assay described below (see, e.g., the Examples section herein), or as measured by any assay available to the skilled artisan.

In some embodiments, this disclosure provides fully human anti-HER2 antibodies that are internalized into cells expressing HER2 and compete with trastuzumab for binding to HER2, as well as methods for using such antibodies for their neutralizing as well as internalizing properties. In preferred embodiments, such antibodies include those referred to herein as 11D7, 2A2, 12E3, 2D7, 3G7, 8C1, and/or the CDRs comprising by such antibodies (see Table 1); the heavy and light chain variable regions thereof (see Table 3); and/or derivatives thereof (e.g., comprising conservative amino acid substitutions thereto (see, e.g., Table 4)).

In some embodiments, this disclosure provides fully human anti-HER2 antibodies that are internalized into cells expressing HER2 and do not compete with trastuzumab for binding to HER2 receptor, as well as methods for studying their internalizing and biological properties, including as ADCs. In preferred embodiments, such antibodies include those referred to herein as 2A1, 7F9, 9E4, 11C9, 12A6; comprising the heavy and light chain variable regions thereof (see Table 3); and/or comprising the CDRs of Table 2; and/or derivatives thereof (e.g., comprising conservative amino acid substitutions thereto (see, e.g., Table 4)).

In some embodiments, this disclosure describes herein is an isolated antibody, or antigen binding fragment thereof, that binds human HER2 and further competes for binding with antibodies 11D7, 2A2, 12E3, 2D7, 3G7, 8C1, 2A1, 7F9, 9E4, 11C9, or 12A6; the heavy and light chain variable regions thereof; and/or a polypeptide comprising the CDRs of such antibodies (see Tables 1 and 2); and/or derivatives thereof (e.g., comprising conservative amino acid substitutions thereto (see, e.g., Table 4)), any of which would be considered an equivalent thereof. Antibodies can also be compared to and identified as equivalents to antibodies 11D7, 2A2, 12E3, 2D7, 3G7, 8C1, 2A1, 7F9, 9E4, 11C9, or 12A6 by ELISA binding to HER-ECD (HER2 extracellular domain), flow cytometric binding to SKBRIII, competition or no competition with trastuzumab for binding to HER2 (e.g., indicating a difference in epitope specificity of the antibodies and/or combinations of CDRs thereof), internalization assays, Octet scouting and epitope binning, a killing assay cytotoxic payload, and/or amino acid and/or nucleotide sequencing. For instance, the antibodies of this disclosure can be identified by referencing the amino acid and/or nucleic acid sequences corresponding to the variability and/or complementarity determining regions (“CDRs”) thereof, and/or the activity thereof (e.g., one antibody blocking of binding of another antibody to HER2). A CDR comprises amino acid residues within the variable region identified in accordance with the definitions of the Kabat, Chothia, the accumulation of both Kabat and Chothia, AbM, contact, and/or conformational definitions or any method of CDR determination well known in the art. antibody modeling software (now Accelrys®), or the “contact definition” of CDRs based on observed antigen contacts described by MacCallum et al., 1996, J. Mol. Biol., 262:732-745. In the “conformational definition” of CDRs, the positions of the CDRs may be identified as the residues that make enthalpic contributions to antigen binding (Makabe et al., 2008, Journal of Biological Chemistry, 283:1156-1166). Still other CDR boundary definitions may not strictly follow one of the above approaches but may nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions. For example, the Chothia and Kabat numbering systems for CDR residues are well known in the art and are further described in the art (see, e.g., Dondelinger, M., et al., Front Immunol 9:2278, 2018). In particular, the mAbs herein include the anti-human HER2 antibodies having the amino acid sequences for the respective CDRs recited in Table 1. For the purposes of this disclosure, the Kabat method has been used to determine the CDRs of the antibodies disclosed herein.

TABLE 1
Antibody Complementarity Determining Regions (CDRs)
Trastuzumab Competitors

| | H-CDR1 | H-CDR2 | H-CDR3 | L-CDR1 | L-CDR2 | L-CDR3 |
|---|---|---|---|---|---|---|
| CDRs | (Kabat) | (Kabat) | (Kabat) | (Kabat) | (Kabat) | (Kabat) |
| 11D7 (SEQ ID | DYAMH (SEQ | GIAWNGGILG | DTGSYSHWYF | RASQSVRSNLA | GASTRAT | QQYNNWPT |
| NOS. 1-6) | ID NO: 1) | YADSVKG | DL (SEQ ID | (SEQ ID NO: | (SEQ ID NO: | (SEQ ID NO: |
| | | (SEQ ID | NO: 3) | 4) | 5 | 6) |
| | | NO: 2) | | | | |
| 2A2 (SEQ ID | DYAMH (SEQ | GISWNGGTLG | DTGSYSHWYF | RASQSVRSNLA | GASTRAT | QQYNNWPT |
| NOS. 1, 7, | ID NO: 1) | YADSVKG | DL (SEQ ID | (SEQ ID NO: | SEQ ID NO: | (SEQ ID NO: |
| 3-6) | | (SEQ ID | NO: 3) | 4) | 5) | 6) |
| | | NO: 7) | | | | |
| 12E3 (SEQ ID | DYAMH (SEQ | GISWNGGTLG | DTGSYSHWYF | RASQSVRSNLA | GASTRAT | QQYNNWPT |
| NOS. 1, 9, | ID NO: 1) | YAGSVKG | DL (SEQ ID | (SEQ ID NO: | (SEQ ID NO: | (SEQ ID NO: |
| 3-6) | | (SEQ TD | NO: 3) | 4) | 5) | |
| | | NO: 9) | | | | |
| 2D7 (SEQ ID | DYAMH (SEQ | GISWNSGTMG | DTGSYSHWYF | RASQSVRTNLA | GASTRAT | QQYNNWPT |
| NOS. 1, 10, | ID NO: 1) | YAGSVKG | DL (SEQ ID | (SEQ ID NO: | (SEQ ID NO: | (SEQ ID NO: |
| 3, 11, 5, 6) | | (SEQ ID | NO: 3) | 11) | 5) | 6) |
| | | NO: 10) | | | | |
| 3G7 (SEQ ID | DYAMH (SEQ | GVSWNGGTMG | DTGSYSHWYF | RASQSVRSNLA | GASTRAT | QQYNNWPT |
| NOS. 1, 12, | ID NO: 1) | YAGSVKG | DL (SEQ ID | (SEQ ID NO: | (SEQ ID NO: | (SEQ ID NO: |
| 3-6) | | (SEQ ID | NO: 3) | 4) | 5) | 6) |
| | | NO: 12) | | | | |
| 8C1 (SEQ ID | DYAMH (SEQ | GISWNGGTLG | DTGSYSHWYF | RASQSVRSNLA | GASTRAT | QQYNNWPT |
| NOS. 1, 13, | ID NO: 1) | YADSVKG | DL (SEQ ID | (SEQ ID NO: | (SEQ ID NO: | (SEQ ID NO: |
| 3-6) | | {SEQ ID | NO: 3) | 4) | 5) | 6) |
| | | NO: 13) | | | | |

TABLE 2
Antibody Complementarity Determining Regions (CDRs)
Trastuzumab Non-Competitors

| | H-CDR1 | H-CDR2 | H-CDR3 | L-CDR1 | L-CDR2 | L-CDR3 |
|---|---|---|---|---|---|---|
| CDRs | (Kabat) | (Kabat) | (Kabat) | (Kabat) | (Kabat) | (Kabat) |
| 2A1 (SEQ ID | SYWMS (SEQ | NIKQDGSEKY | EVGPGDY | RASQSISNWVA | TASSLES | QQYYSYSPT |
| NOS. 14-19) | ID NO: 14) | YVDSVKG | (SEQ ID | (SEQ ID NO: | (SEQ ID NO: | (SEQ ID NO: |
| | | (SEQ ID | NO: 16) | 17) | 18) | 19) |
| | | NO: 15) | | | | |
| 7F9 (SEQ ID | SSAVQ (SEQ | WIVVGSGNTN | GYYGSGSPYY | RASQGISSWLA | AASSLQS | QQVDSFPFT |
| NOS. 20-25) | ID NO: 20) | YAQKFQE | YYYGMDV | (SEQ ID NO: | (SEQ ID NO: | (SEQ ID NO: |
| | | (SEQ ID | (SEQ ID | 23) | 24) | 25) |
| | | NO: 21) | NO: 22) | | | |
| 9E4 (SEQ ID | DYYMS (SEQ | YISSSGITIY | DGQQLADYYY | RASQSVSSSYL | GASSRAT | QQYGSSLT |
| NOS. 26-31) | ID NO: 26) | YADSVKG | YGMDV (SEQ | T (SEQ ID | (SEQ ID NO: | (SEQ ID NO: |
| | | (SEQ ID | ID NO: 28) | NO: 29) | 30) | 31) |
| | | NO: 27) | | | | |
| 11C9 (SEQ ID | SYGMH (SEQ | VIWYDGGNKD | VGQLAHYYYG | RSSQSLLHSNG | LGSNRAS | MQALQSPPT |
| NOS. 32-37) | ID NO: 32) | YADSVKG | MDV (SEQ | YNYLD (SEQ | (SEQ ID NO: | (SEQ ID NO: |
| | | (SEQ ID | ID NO: 34) | ID NO: 35) | 36) | 37) |
| | | NO: 33) | | | | |
| 12A6 (SEQ ID | SYGMH (SEQ | VIWYDGSDDY | ELVRGASFDY | RSSQSLLHSNG | LGSNRAS | MQALQTPLT |
| NOS. 32, 38, | ID NO: 32) | YADSVKG | (SEQ ID | YNYLD (SEQ | (SEQ ID NO: | (SEQ ID NO: |
| 39, 35, 36, 39) | | (SEQ ID | NO: 39) | ID NO: 35) | 36) | 40) |
| | | NO: 30) | | | | |

The antibodies can further include the complete heavy and/or light chain of the antibodies shown in Table 3, and/or a derivative thereof, that include the CDRs shown in Tables 1 and 2. In some preferred embodiments, such antibodies can have the amino acid sequences of the variable heavy (“VH”) including heavy chain framework regions (HFR1, HFR2, HFR3, HFR4 (included in the sequences below and listed separately in the Examples section)) or variable light (“VL”) polypeptides including light chain framework regions (LFR1, LFR2, LFR3, LFR4 (included in the sequences below below listed separately in the Examples section)) (VH or VL “chains”, respectively) shown below for the antibody or equivalent thereof shown in Table 3:

TABLE 3

| Antibody | |
|---|---|
| VH or VL | Amino Acid Sequence (CDRs highlighted in underline) |
| 11D7 VH | EVQLVESGGGLVQPGRSLRLSCAASGFTFDDYAMHWVRQAPGKGLEWVSGIAWNGGIL |
| | GYADSVKGRFTISRDNAKNSLYLQMNSLRAEDTALYYCAKDTGSYSHWYFDLWGRGTL |
| | VTVSS (SEQ ID NO: 41) |
| 11D7 VL | EIVMTQSPATLSVSPGERATLSCRASQSVRSNLAWYQQKPGQAPRLLIYGASTRATGI |
| | PARESGSGSGTEFTLTISSLQSEDFAVYCCQQYNNWPTFGQGTKVEIK (SEQ ID |
| | NO: 42) |
| 2A2 VH | EVQLVESGGGLVQPGRSLRLSCAASGFTFDDYAMHWVRQAPGKGLEWVSGISWNGGTL |
| | GYADSVKGRFTISRDNAKNSLYLQMNSLRAEDTALYYCAKDTGSYSHWYFDLWGRGTL |
| | VTVSS (SEQ ID NO: 43) |
| 2A2 VL | EIVMTQSPATLSVSPGERATLSCRASQSVRSNLAWYQQKPGQAPRLLIYGASTRATGI |
| | PARFSGSGSGTEFTLTISSLHSEDFAVYYCQQYNNWPTFGQGTKVEIK (SEQ ID |
| | NO: 44) |
| 12E3 VH | EVQLVGSGGGLVQPGRSLRISCAASGFTFDDYAMHWVRQAPGKGLEWVSGISWNGGTL |
| | GYAGSVKGRFTISRDNAKNSLYLQMNSLRAEDTALYYCAKDTGSYSHWYFDLWGRGTL |
| | VTVSS (SEQ ID NO: 45) |
| 12E3 VL | EIVMTQSPATLSVSPGERATLSCRASQSVRSNLAWYQQKPGQAPRLLIYGASTRATGI |
| | PARCSGSGSGTEFTLTISSLQSEDFAVYYCQQYNNWPTFGQGTKVEIK (SEQ ID |
| | NO: 46) |
| 2D7 VH | EVQLVESGGGLVQPGRSLRISCAASGFTFDDYAMHWVRQAPGKGLEWVSGISWNSGTM |
| | GYAGSVKGRFTISRDNAKNSLYLQMNSLRAEDTALYYCAKDTGSYSHWYFDLWGRGTL |
| | VTVSS (SEQ ID NO: 47) |
| 2D7 VL | EIVMTQSPATLSVSPGERATLSCRASQSVRTNLAWYQQKPGQAPRLLIYGASTRATGI |
| | PARESGSGSGTEFTLTISSLQSEDFAVYYCQQYNNWPTFGQGTKVEIK (SEQ ID |
| | NO: 48) |
| 3G7 VH | EVQLVESGGGLVQPGRSLRLSCAASGFTFDDYAMHWVRQAPGKGLEWVSGVSWNGGTM |
| | GYAGSVKGRFTISRDNAKNSLYLQMNSLRAEDTALYYCAKDTGSYSHWYFDLWGRGTL |
| | VTVSS (SEQ ID NO: 49) |
| 3G7 VL | EIVMTQSPATLSVSPGERATLSCRASQSVRSNLAWYQQKPGQAPRLLIYGASTRATGI |
| | PGRFSGSGSGTEFILTISSLQSEDFAVYYCQQYNNWPTFGQGTKVEIK (SEQ ID |
| | NO: 50) |
| 8C1 VH | EVQLVESGGGLVQPGRSLRLSCTASGFTFDDYAMHWVRQAPGKGLEWVSGISWNGGTL |
| | GYADSVKGRFTISRDNAKNSLYLQMNSLRAEDTALYYCAKDIGSYSHWYFDLWGRGTL |
| | VTVSS (SEQ ID NO: 51) |
| 8C1 VL | EIVMTQSPATLSVSPGERATLSCRASQSVRSNLAWYQQKPGQAPRLLIYGASTRAT |
| | GIPARFSGSGSGTEFTLTISSLHSEDFAVYYCQQYNNWPTFGQGTKVEIK (SEQ |
| | ID NO: 52) |
| 2A1 VH | EVQLVESGGGLVQPGGSLRLSCAASGFTFSSYWMSWVRQAPGKGLEWVANIKQDGSEK |
| | YYVDSVKGRFTISRDNAKNSLYLQMNSLRAEDTAVYYCAREVGPGDYWGQGTLVTVSS |
| | (SEQ ID NO: 53) |
| 2A1 VL | DIQMTQSPSTLSASVGDRVTITCRASQSISNWVAWYQQKPGKAPKLLIHTASSLESGV |
| | PSRESGSGSGTEFTLTISSLQPDDFATYYCQQYYSYSPTEGQGTKVEIK (SEQ ID |
| | NO: 54) |
| 7F9 VH | QMQLVQSGPEVKKPGTSVKVSCKASGFTFTSSAVQWVRQARGQRLEWIGWIVVGSGNT |
| | NYAQKFQERVTITRDMSTSTAYMELSSLRSEDTAVYYCAAGYYGSGSPYYYYYGMDVW |
| | GQGTTVTVSS (SEQ ID NO: 55) |
| 7F9 VL | DIQMTQSPSSVSASVGDRVTITCRASQGISSWLAWYQQKSGKAPKLLIYAASSLQSGV |
| | PSRFSGSGSGTDFTLTINSLQPEDFATYYCQQVDSFPFTFGGGTKVESK (SEQ ID |
| | NO: 56) |
| 9E4 VH | QVQLVESGGGLVKPGGSLRLSCAASGFTFSDYYMSWLRQAPGRGLEWVSYISSSGITI |
| | YYADSVKGRFTISRDNAKNSLYLQMNSLRAEDTAVYYCARDGQQLADYYYYGMDVWGQ |
| | GTTVTVSS (SEQ ID NO: 57) |
| 9E4 VL | EIVLTQSPGTLSLSPGERATLSCRASQSVSSSYLTWYQQKPGQAPRLLIYGASSRATG |
| | IPDRESGSGSGIDFTITISRLEPEDFAVYYCQQYGSSLTFGGGTKVEIK (SEQ ID |
| | NO: 58) |
| 11C9 VH | QVQLVESGGGVVQPGRSLRISCAASGFTFSSYGMHWVRQAPGKGLEWVAVIWYDGGNK |
| | DYADSVKGRFTISRDNSKNTLYLQMNSLRAEDTAVYYCARVGQLAHYYYGMDVWGQGT |
| | TVIVSS (SEQ ID NO: 59) |
| 11C9 VL | DIVMTQSPLSLPVTPGEPASISCRSSQSLLHSNGYNYLDWYLQKPGQSPQLLIYLGSN |
| | RASGVPDRFSGSGSGTDFTLKISRVEAEDVGVYYCMQALQSPPTFGQGTKVEIK |
| | (SEQ ID NO: 60) |
| 12A6 VH | QVQLVESGGGVVQPGRSLRISCAASGFTFSSYGMHWVRQAPGKGLEWVAVIWYDGSDD |
| | YYADSVKGRFTISRDNSKNTLYLQMNSLRAEDTAVYYCARELVRGASFDYWGQGTLVT |
| | VSS (SEQ ID NO: 61) |
| 12A6 VL | EILMTQSPLSLPVTPGEPATISCRSSQSLLHSNGYNYLDWYLQKPGQSPQLLIYLGSN |
| | RASGVPDRESGSGSGTDFTLRISRVEAEDVGVYYCMQALQTPLTFGGGTKVEIK |
| | (SEQ ID NO: 62) |

In preferred embodiments, and in accordance with Table 3, the 11D7 antibody comprises SEQ ID NOS. 41 and 42; the 2A2 antibody comprises SEQ ID NOS. 43 and 44; the 12E3 antibody comprises SEQ ID NOS. 45 and 46; the 2D7 antibody comprises SEQ ID NOS. 47 and 48; the 3G7 antibody comprises SEQ ID NOS. 49 and 50; the 8C1 antibody comprises SEQ ID NOS. 51 and 52; the 2A1 antibody comprises SEQ ID NOS. 53 and 54; the 7F9 antibody comprises SEQ ID NOS. 55 and 56; the 9E4 antibody comprises SEQ ID NOS. 57 and 58; the 11C9 antibody comprises SEQ ID NOS. 59 and 60; and the 12A6 antibody comprises SEQ ID NOS. 61 and 62.

For polypeptide sequences, “conservatively modified variants” include individual substitutions, deletions or additions to a polypeptide sequence which result in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles. In some embodiments, the term “conservative sequence modifications” are used to refer to amino acid modifications that do not significantly affect or alter the binding characteristics of the antibody containing the amino acid sequence. It is preferred that the antibody, or the antigen binding fragment thereof, comprises one or more amino acid sequences having at least 70%, at least 75%, at least 80%, at least 85%, at least 88%, at least 90%, at least 92%, at least 95%, at least 96%, at least 97%, at least 98% or at least 99% identity to at least one of SEQ ID NOs. 1-62 (i.e., the CDR sequences, the VH sequence and/or the VL sequences shown in Tables 1-3). In some embodiments, an equivalent to an 11D7, 2A2, 12E3, 2D7, 3G7, 8C1, 2A1, 7F9, 9E4, 11C9, or 12A6 antibody includes a derivative of one or more of the CDRs of the 11D7, 2A2, 12E3, 2D7, 3G7, 8C1, 2A1, 7F9, 9E4, 11C9, or 12A6 antibodies, preferably including up to three (3) conservative amino acid substitutions of the CDRs thereof (see Table 4), provided the derivatives maintain their ability to bind to HER2. In some embodiments, an equivalent to an 11D7, 2A2, 12E3, 2D7, 3G7, 8C1, 2A1, 7F9, 9E4, 11C9, or 12A6 antibody includes a derivative of one or more of the VH and/or VL chains of the 11D7, 2A2, 12E3, 2D7, 3G7, 8C1, 2A1, 7F9, 9E4, 11C9, or 12A6 antibodies, preferably including up to ten conservative amino acid substitutions outside of the CDRs thereof (see Table 4), provided the derivatives maintain the ability to bind to HER2. In preferred embodiments, any such substitutions allow for conjugation of the antibodies, or do not interfere with conjugation of the antibodies, to one or more detectable label(s), cytotoxic agent(s), and/or other payload (e.g., to provide a bi-specific antibody).

Amino acid substitutions considered conservative and non-conservative using standard three letter or other abbreviations for amino acids as would be understood by those of skill in the art are shown below in Table 4:

TABLE 4

| Original | Exemplary Conservative | Preferred Conservative |
|---|---|---|
| Amino | Substitutions of | Substitution of |
| Acid | the Original | the Original |
| Residue | Amino Acid Residue | Amino Acid Residue |
| Ala | Val, Leu, Ile | Val |
| Arg | Lys, Gln, Asn | Lys |
| Asn | Gln | Gln |
| Asp | Glu | Glu |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, | Leu |
| | Norleucine | |
| Leu | Norleucine, Ile, Val, Met, | Ile |
| | Ala, Phe | |
| Lys | Arg, 1,4 Diamino-butyric | Arg |
| | Acid, Gln, Asn | |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, | Leu |
| | Norleucine | |

In certain embodiments, a nucleic acid molecule encoding one or more antibodies described herein may be inserted into one or more expression vectors, as discussed below in greater detail. In such embodiments, the antibody may be encoded by nucleotides corresponding to the amino acid sequence. The particular combinations of nucleotides (codons) that encode the various amino acids (AA) are well known in the art, as described in various references used by those skilled in the art (e.g., Lewin, B. Genes V, Oxford University Press, 1994). The nucleotide sequences encoding the amino acids of said antibodies may be ascertained with reference to Table 5, for example. Nucleic acid variants may use any combination of nucleotides that encode the antibody.

TABLE 5
Codons Encoding Amino Acids (AA)

| AA | Codon | AA | Codons | AA | Codons | AA | Codons |
|---|---|---|---|---|---|---|---|
| Phe (F) | TTT | Ser (S) | TCT | Tyr (Y) | TAT | Cys (C) | TGT |
| | TTC | | TCC | | TAC | | TGC |
| Leu (L) | TTA | | TCA | TERM | TAA | TERM | TGA |
| | TTG | | TCG | | TAG | Trp (W) | TGG |
| | CTT | Pro (P) | CCT | His (H) | CAT | Arg (R) | CGT |
| | CTC | | CCC | | CAC | | CGC |
| | CTA | | CCA | Gln (Q) | CAA | | CGA |
| | CTG | | CCG | | CAG | | CGG |
| Ile (I) | ATT | Thr (T) | ACT | Asn (N) | AAT | Ser (S) | AGT |
| | ATC | | ACC | | AAC | | AGC |
| | ATA | | ACA | Lys (K) | AAA | Arg (R) | AGA |
| Met (M) | ATG | | ACG | | AAG | | AGG |
| Val (V) | GTT | Ala (A) | GCT | Asp (D) | GAT | Gly (G) | GGT |
| | GTC | | GCC | | GAC | | GGC |
| | GTA | | GCA | Glu (E) | GAA | | GGA |
| | GTG | | GCG | | GAG | | GGG |

In preferred embodiments, the VH and VL amino acid sequences are encoded by the nucleotide sequences shown in Table 6:

TABLE 6

| Antibody | |
|---|---|
| VH or VL | Preferred Nucleotide Sequence |
| 11D7 VH | gaagtgcagctggtggagtctgggggaggcttggtacagcctggcaggtccctgag |
| | actctcctgtgcagcctctggattcacctttgatgattatgccatgcactgggtcc |
| | ggcaagctccagggaagggcctggagtgggtctcaggtattgcttggaatggtggt |
| | atcttaggctatgcggactctgtgaagggccgattcaccatctccagagacaacgc |
| | caagaactccctgtatctgcaaatgaacagtctgagagctgaggacacggccttgt |
| | attactgtgcaaaagatacggggagttattctcactggtacttcgatctctggggc |
| | cgtggcaccctggtcactgtctcctcag (SEQ ID NO: 63) |
| 11D7 VL | gaaatagtgatgacgcagtctccagccaccctgtctgtgtctccaggggaaagagc |
| | caccctctcctgcagggccagtcagagtgttaggagcaacttagcctggtatcagc |
| | agaaacctggccaggctcccaggctcctcatctatcgtgcatccaccagggccact |
| | ggtatcccagccaggttcagtggcagtgggtctgggacagagttcactctcaccat |
| | cagcagcctgcagtctgaagattttgcagtttattgctgtcagcagtataataact |
| | ggcctacgttcggccaagggaccaaggtggaaatcaaac (SEQ ID NO: 64) |
| 2A2 VH | gaagtgcagttggtggagtctgggggaggcttggtacagcctggcaggtccctgag |
| | actctcctgtgcagcctctggattcacctttgatgattatgccatgcactgggtcc |
| | ggcaagctccagggaagggcctggagtgggtctcaggtattagttggaatggtggt |
| | accttaggctatgcggactctgtgaagggccgattcaccatctccagagacaacgc |
| | caagaactccctgtatctgcaaatgaacagtctgagagctgaggacacggccttgt |
| | attactgtgcaaaagatacggggagttattctcactggtacttcgatctctggggc |
| | cgtggcaccctggtcactgtctcctcag (SEQ ID NO: 65) |
| 2A2 VL | gaaatagtgatgacgcagtctccagccaccctgtctgtgtctccaggggaaagagc |
| | caccctctcctgcagggccagtcagagtgttaggagcaacttagcctggtatcagc |
| | agaaacctggccaggctcccaggctcctcatctatggtgcatccaccagggccact |
| | ggtatcccagccaggttcagtggcagtgggtctgggacagagttcactctcaccat |
| | tagcagcctgcactctgaagattttgcagtttattactgtcagcagtataacaact |
| | ggcctacgttcggccaagggaccaaggtggaaatcaaac (SEQ ID NO: 66) |
| 12E3 VH | gaagtgcagctggtggggtctgggggaggtttggtacagcctggcaggtccctgag |
| | actctcctgtgcagcctctggattcacctttgatgattatgccatgcactgggtcc |
| | ggcaagctccagggaagggcctggagtgggtctcaggtattagttggaatggtggt |
| | acgttgggctatgcgggctctgtgaagggccgattcaccatctccagagacaacgc |
| | caagaactccctgtatctgcaaatgaacagtctgagagctgaggacacggccttgt |
| | attactgtgcaaaagatacggggagttattctcactggtacttcgatctctggggc |
| | cgtggcaccctggtcactgtctcctcag (SEQ ID NO: 67) |
| 12E3 VL | gaaatagtgatgacgcagtctccagccaccctgtctgtgtctccaggggaaagagc |
| | caccctctcctgcagggccagtcagagtgttagaagcaacttagcctggtaccagc |
| | agaaacctggccaggctcccaggctcctcatctatggtgcatccaccagggccact |
| | ggtatcccagccaggtgcagtggcagtgggtctgggacagagttcactctcaccat |
| | cagcagcctgcagtctgaggattttgcagtttattactgtcagcagtataataact |
| | ggcctacgttcggccaagggaccaaggtggaaatcaaac (SEQ ID NO: 68) |
| 2D7 VH | gaagtgcagctggtggagtctgggggaggtttggtacagcctggcaggtccctgag |
| | actctcctgtgcagcctctggattcacctttgatgattatgccatgcactgggtcc |
| | ggcaagctccagggaagggcctggagtgggtctcaggtattagttggaatagtggt |
| | accatgggctatgcgggctctgtgaagggccgattcaccatctccagagacaacgc |
| | caagaactccctgtatctgcaaatgaacagtctgagagctgaggacacggccttgt |
| | attactgtgcaaaagatacggggagttattctcactggtacttcgatctctggggc |
| | cgtggcaccctggtcactgtctcctcag (SEQ ID NO: 69) |
| 2D7 VL | gaaatagtgatgacgcagtctccagccaccctgtctgtgtctccaggggaaagagc |
| | caccctctcctgcagggccagtcagagtgttaggaccaacttagcctggtaccagc |
| | agaaacctggccaggctcccaggctcctcatctatggtgcatccaccagggccact |
| | ggcatcccagccaggttcagtggtagtgggtctgggacagagttcactctcaccat |
| | cagcagcctgcagtctgaagattttgcagtttattactgtcagcagtataataact |
| | ggcctacgttcggccaagggaccaaggtggaaatcaaac (SEQ ID NO: 70) |
| 3G7 VH | gaagtgcagctggtggagtctgggggaggtttggtacagcctggcaggtccctgag |
| | actctcctgtgcagcctctggattcacctttgatgattatgccatgcactgggtcc |
| | ggcaagctccagggaagggcctggagtgggtctcaggtgttagttggaatggtggt |
| | accatgggctatgcgggctctgtgaagggccgattcaccatctccagagacaacgc |
| | caagaactccctgtatctgcaaatgaacagtctgagagctgaggacacggccttgt |
| | attactgtgcaaaagatacggggagttattctcactggtacttcgatctctggggc |
| | cgtggcaccctggtcactgtctcctcag (SEQ ID NO: 71) |
| 3G7 VL | gaaatagtgatgacgcagtctccagccaccctgtctgtgtctccaggggaaagagc |
| | caccctctcctgcagggccagtcagagtgttaggagcaacttagcctggtaccagc |
| | agaaacctggccaggctcccaggctcctcatctatggtgcatccaccagggccact |
| | ggtatcccaggcaggttcagtggcagtgggtctgggacagagttcattctcaccat |
| | cagcagcctgcagtctgaagattttgcagtttattactgtcagcagtataataact |
| | ggcctacgttcggccaagggaccaaggtggaaatcaaac (SEQ ID NO: 72) |
| 8C1 VH | gaagtgcagttggtggagtctgggggaggcttggtacagcctggcaggtccctgag |
| | actctcctgtacagcctctggattcacctttgatgattatgccatgcactgggtcc |
| | ggcaagctccagggaagggcctggagtgggtctcaggtattagttggaatggtggt |
| | accttaggctatgcggactctgtgaagggccgattcaccatctccagagacaacgc |
| | caagaactccctgtatctgcaaatgaacagtctgagagctgaggacacggccttgt |
| | attactgtgcaaaagatacggggagttattctcactggtacttcgatctctggggc |
| | cgtggcaccctggtcactgtctcctcag (SEQ ID NO: 73) |
| 8C1 VL | gaaatagtgatgacgcagtctccagccaccctgtctgtgtctccaggggaaagagc |
| | caccctctcctgtagggccagtcagagtgttaggagcaacttagcctggtatcagc |
| | agaaacctggccaggctcccaggctcctcatctatggtgcatccaccagggccact |
| | ggtatcccagccaggttcagtggcagtgggtctgggacagagttcactctcaccat |
| | tagcagcctgcactctgaagattttgcagtttattactgtcagcagtataacaact |
| | ggcctacgttcggccaagggaccaaggtggaaatcaaac (SEQ ID NO: 74) |
| 2A1 VH | gaggtgcagctggtggagtctgggggaggcttggtccagcctggggggtccctgag |
| | actctcctgtgcagcctctggattcacctttagtagctattggatgagctgggtcc |
| | gccaggctccagggaaggggctggagtgggtggccaacataaagcaagatggaagt |
| | gagaaatactatgtggactctgtgaagggccgattcaccatctccagagacaacgc |
| | caagaactcactgtatctgcaaatgaacagcctgagagccgaggacacggctgtgt |
| | attactgtgcgagagaggtggggcccggggactactggggccagggaaccctggtc |
| | accgtctcctcag (SEQ ID NO: 75) |
| 2A1 VL | gacatccagatgacccagtctccttccaccctgtctgcatctgtaggagacagagt |
| | caccatcacttgccgggccagtcagagtattagtaactgggtggcctggtatcagc |
| | agaaaccagggaaagcccctaagctcctgatccatacggcatctagcttagaaagt |
| | ggggtcccatcaaggttcagcggcagtggatctgggacagaattcactctcaccat |
| | cagcagcctgcagcctgatgattttgcaacttattactgccaacagtattatagtt |
| | attctccgacgttcggccaagggaccaaggtggaaatcaaac (SEQ ID NO: 76) |
| 7F9 VH | caaatgcagctggtgcagtctgggcctgaggtgaagaagcctgggacctcagtgaa |
| | ggtctcctgcaaggcttctggattcacctttactagctctgctgtgcagtgggtgc |
| | gacaggctcgtggacaacgccttgagtggataggatggatcgtcgttggcagtggt |
| | aacacaaactacgcacagaagttccaggaaagagtcaccattaccagggacatgtc |
| | cacaagcacagcctacatggagctgagcagcctgagatccgaggacacggccgtgt |
| | attactgtgcggcggggtactatggttcggggagtccttactactactactacggt |
| | atggacgtctggggccaagggaccacggtcaccgtctcctcag (SEQ ID NO: |
| | 77) |
| 7F9 VL | gacatccagatgacccagtctccatcttccgtgtctgcatctgtaggagacagagt |
| | caccatcacttgtcgggcgagtcagggtattagcagctgcttagcctggtatcagc |
| | agaaatcagggaaagcccctaagctcctgatctatgctgcatctagtttgcaaagt |
| | ggggtcccatcaaggttcagcggcagtggatctgggacagatttcactctcaccat |
| | caacagcctgcagcctgaagattttgcaacttactattgtcaacaggttgacagtt |
| | tcccgttcactttcggcggagggaccaaggtggagagcaaac (SEQ ID NO: 78) |
| 9E4 VH | caggtgcagctggtggagtctgggggaggcttggtcaagcctggagggtccctgag |
| | actctcctgtgcagcctctggattcaccttcagtgactactacatgagctggctcc |
| | gccaggctccagggagggggctggagtgggtttcatacattagtagtagtggtatt |
| | accatatactacgcagactctgtgaagggccgattcaccatctccagggacaacgc |
| | caagaactcactgtatctgcaaatgaacagcctgagagccgaggacacggctgtgt |
| | attactgtgcgagagatggacagcagctggccgactactactactacggtatggac |
| | gtctggggccaagggaccacggtcaccgtctcctcag (SEQ ID NO: 79) |
| 9E4 VL | gaaattgtgttgacgcagtctccaggcaccctgtctttgtctccaggggaaagagc |
| | caccctctcctgcagggccagtcagagtgttagcagcagctacttaacctggtacc |
| | agcagaaacctggccaggctcccaggctcctcatctatggtgcatccagcagggcc |
| | actggcatcccagacaggttcagtggcagtgggtctgggacagacttcactctcac |
| | catcagcagactggagcctgaagattttgcagtgtattactgtcagcagtatggta |
| | gctcactcactttcggcggagggaccaaggtggagatcaaac (SEQ ID NO: 80) |
| 11C9 VH | caggtgcagctggtggagtctgggggaggcgtggtccagcctgggaggtccctgag |
| | actctcctgtgcagcgtctggattcacgttcagtagctatggcatgcactgggtcc |
| | gccaggctccaggcaaggggctggagtgggtggcagttatatggtatgatggaggt |
| | aataaagactatgcagactccgtgaagggccgattcaccatctccagagacaattc |
| | caagaacacgctgtatctgcaaatgaacagcctgagagccgaggacacggctgtgt |
| | attactgtgcgagagtagggcagctggcacactactactacggtatggacgtctgg |
| | ggccaagggaccacggtcaccgtctcctcag (SEQ ID NO: 81) |
| 11C9 VL | gatattgtgatgactcagtctccactctccctgcccgtcacccctggagagccggc |
| | ctccatctcctgcaggtctagtcagagcctcctgcatagtaatggatacaactatt |
| | tggattggtacctgcagaagccagggcagtctccacagctcctgatctatttgggt |
| | tctaatcgggcctccggggtccctgacaggttcagtggcagtggatcaggcacaga |
| | ttttacactgaaaatcagcagagtggaggctgaggatgttggggtttattactgca |
| | tgcaagctctacaaagtcctccgacgttcggccaagggaccaaggtggaaatcaaa |
| | C (SEQ ID NO: 82) |
| 12A6 VH | caggtgcaactggtggagtctgggggaggcgtggtccagcctgggaggtccctgag |
| | actctcctgtgcagcgtctggcttcaccttcagtagctatggcatgcactgggtcc |
| | gccaggctccaggcaaggggctggagtgggtggcagttatatggtatgatggaagt |
| | gatgactactatgcagactccgtgaagggccgattcaccatctccagagacaattc |
| | caagaacacgctgtatctgcaaatgaacagcctgagagccgaggacacggctgtgt |
| | attactgtgcgagagagctggttcggggagcctcctttgactactggggccaggga |
| | accctggtcaccgtctcctcag (SEQ ID NO: 83) |
| 12A6 VL | gaaattttgatgactcagtctccactctccctgcccgtcacccctggagagccggc |
| | caccatctcctgcaggtctagtcagagcctcctccatagtaatggatacaactatt |
| | tggattggtacctgcagaagccagggcagtctccacagctcctgatctatttgggt |
| | tctaatcgggcctccggggtccctgacaggttcagtggcagtggatcaggcacaga |
| | ttttacactgagaatcagcagagtggaggctgaggatcttggggtttattactgca |
| | tgcaagctctacaaactccgctcactttcggcggagggaccaaggtggagatcaaa |
| | C (SEQ ID NO: 84) |

Those of ordinary skill in the art understand that the nucleotide sequence encoding a particular amino acid sequence of an equivalent to an 11D7, 2A2, 12E3, 2D7, 3G7, 8C1, 2A1, 7F9, 9E4, 11C9, or 12A6 antibody may be easily derived from the amino acid sequence of any of SEQ ID NOS. 63-84 and the information presented in Table 4. For instance, it may be deduced from the amino acid sequence DYAMH (SEQ ID NO: 1) and the information presented in Table 4 that the amino acid sequence may be encoded by the nucleotide sequence GAT TAT GCT ATG CAT (SEQ ID NO.: 84). Those of ordinary skill in the art would understand that nucleotide sequences encoding SEQ ID NOS. 1-62 and derivatives thereof may be deduced in the same way, and such nucleotide sequences are contemplated herein. This disclosure also provides an expression vector including an isolated nucleic acid comprising and/or consisting of such nucleotide sequences (in preferred embodiments any of SEQ ID NOS. 63-84 or derivatives thereof) as well as host cells (e.g., a cell line) containing such an expression vector.

This disclosure provides, in preferred embodiments an isolated antibody or antigen binding fragment thereof, comprising: a) a heavy chain variable region comprising the CDR sequences SEQ ID NOs: 1, 2, and 3 and a light chain variable region comprising CDR sequences SEQ ID NOs: 4, 5, and 6 (antibody 11D7; Table 1); b) a heavy chain variable region comprising the CDR sequences SEQ ID NOs: 1, 7 and 3 and a light chain variable region comprising CDR sequences SEQ ID NOs: 4, 5, and 6 (antibody 2A2; Table 1); c) a heavy chain variable region comprising the CDR sequences SEQ ID NOs: 1, 9 and 3 and a light chain variable region comprising CDR sequences SEQ ID NOs: 4, 5, and 6 (antibody 12E3; Table 1); d) a heavy chain variable region comprising the CDR sequences SEQ ID NOs: 1, 10 and 3 and a light chain variable region comprising CDR sequences SEQ ID NOs: 4, 5, and 6 (antibody 2D7; Table 1); e) a heavy chain variable region comprising the CDR sequences SEQ ID NOs: 1, 12, and 3 and a light chain variable region comprising CDR sequences SEQ ID NOs: 4, 5, and 6 (antibody 3G7; Table 1); f) a heavy chain variable region comprising the CDR sequences SEQ ID NOs: 1, 13, and 3 and a light chain variable region comprising CDR sequences SEQ ID NOs: 4, 5, and 6 (antibody 8C1; Table 1); g) a heavy chain variable region comprising the CDR sequences SEQ ID NOs: 14, 15 and 16 and a light chain variable region comprising CDR sequences SEQ ID NOs: 17, 18, and 19 (antibody 2A1; Table 2); h) a heavy chain variable region comprising the CDR sequences SEQ ID NOs: 20, 21 and 22 and a light chain variable region comprising CDR sequences SEQ ID NOs: 23, 24, and 25 (antibody 7F9; Table 2); i) a heavy chain variable region comprising the CDR sequences SEQ ID NOs: 26, 27, and 28 and a light chain variable region comprising CDR sequences SEQ ID NOs: 29, 30, and 31 (antibody 9E4; Table 2); j) a heavy chain variable region comprising the CDR sequences SEQ ID NOs: 32, 33, 34 and a light chain variable region comprising CDR sequences SEQ ID NOs: 35, 36 and 37 (antibody 11C9; Table 2); k) a heavy chain variable region comprising the CDR sequences SEQ ID NOs: 32, 38, and 39 and a light chain variable region comprising CDR sequences SEQ ID NOs: 35, 36 and 40 (antibody 12A6; Table 2); l) a heavy chain variable region comprising SEQ ID NO:41 and a light chain variable region comprising SEQ ID NOs: 42 (antibody 11D7; Table 3); m) a heavy chain variable region comprising SEQ ID NO:43 and a light chain variable region comprising SEQ ID NOs: 44 (antibody 2A2; Table 3); n) a heavy chain variable region comprising SEQ ID NO:45 and a light chain variable region comprising SEQ ID NOs: 46 (antibody 12E3; Table 3); o) a heavy chain variable region comprising SEQ ID NO:47 and a light chain variable region comprising SEQ ID NOs: 48 (antibody 2D7; Table 3); p) a heavy chain variable region comprising SEQ ID NO: 49 and a light chain variable region comprising SEQ ID NOs: 50 (antibody 3G7; Table 3); q) a heavy chain variable region comprising SEQ ID NO:51 and a light chain variable region comprising SEQ ID NOs: 52 (antibody 8C1; Table 3); r) a heavy chain variable region comprising SEQ ID NO:53 and a light chain variable region comprising SEQ ID NOs: 54 (antibody 2A1; Table 3); s) a heavy chain variable region comprising SEQ ID NO:55 and a light chain variable region comprising SEQ ID NOs: 56 (antibody 7F9; Table 3); t) a heavy chain variable region comprising SEQ ID NO:57 and a light chain variable region comprising SEQ ID NOs: 58 (antibody 9E4; Table 3); u) a heavy chain variable region comprising SEQ ID NO: 59 and a light chain variable region comprising SEQ ID NOs: 60 (antibody 11C9; Table 3); v) a heavy chain variable region comprising SEQ ID NO:61 and a light chain variable region comprising SEQ ID NOs: 62 (antibody 12A6; Table 3); w) a heavy chain variable region and corresponding a light chain variable region encoded by any of polynucleotides of SEQ ID NOS. 63-84 (Table 6); and/or a conservatively substituted derivative thereof optionally comprising up to three amino acid substitutions in one or more CDRs thereof and/or up to ten amino acid substitutions of a heavy and/or light chain thereof; wherein the antibody or antigen binding fragment thereof specifically binds to human HER2. In some preferred embodiments, this disclosure provides that is internalized into a cell that expresses HER2 in vitro and/or in vivo. In some preferred embodiments, this disclosure provides that competes with trastuzumab for binding to HER2 receptor on the cell (e.g., antibodies 11D7, 2A2, 12E3, 2D7, 3G7, and 8C1). In some preferred embodiments, this disclosure provides that does not compete with trastuzumab for binding to HER2 receptor on the cell (e.g., antibodies 2A1, 7F9, 9E4, 11C9, and 12A6, or in combination with an antibody such as trastuzumab). In some preferred embodiments, this disclosure provides that is an isolated monoclonal antibody, such as a human monoclonal antibody. This disclosure also provides combinations of such antibodies, and compositions of the same (preferably pharmaceutically acceptable compositions) including, but not limited to, at least one antibody that bind to distinct epitopes on human HER2 such as (preferably) any two or more of antibodies 11D7, 2A2, 12E3, 2D7, 3G7, 8C1, 2A1, 7F9, 9E4, 11C9, and 12A6. In some embodiments, the combination can include at least one antibody that competes with trastuzumab for binding to HER2 receptor on the cell (e.g., antibodies 11D7, 2A2, 12E3, 2D7, 3G7, and 8C1) and at least one antibody that does not compete with trastuzumab for binding to HER2 receptor on the cell (e.g., antibodies 2A1, 7F9, 9E4, 11C9, and 12A6). In some embodiments, the combination(s) can include trastuzumab. In some preferred embodiments, the combination of antibodies can be prepared as a composition (preferably a pharmaceutically acceptable composition).

In some preferred embodiments, the antibody is derived from a human antibody, human IgG, human IgG1, human IgG2, human IgG2a, human IgG2b, human IgG3, human IgG4, human IgM, human IgA, human IgA1, human IgA2, human IgD, human IgE, canine antibody, canine IgGA, canine IgGB, canine IgGC, canine IgGD, chicken antibody, chicken IgA, chicken IgD, chicken IgE, chicken IgG, chicken IgM, chicken IgY, goat antibody, goat IgG, mouse antibody, mouse IgG, pig antibody, rat antibody, llaman antibody, alpacan antibody, shark antibody and a camel antibody. In some preferred embodiments, this disclosure provides a derivative of an antibody disclosed herein, optionally selected from the group consisting of an F_{ab}, F_{ab2}, Fab′ single chain antibody, Fv, single chain, mono-specific antibody, bispecific antibody, trimeric antibody, multi-specific antibody, multivalent antibody, chimeric antibody, canine-human chimeric antibody, canine-mouse chimeric antibody, antibody comprising a canine Fc, humanized antibody, human antibody, caninized antibody, CDR-grafted antibody, shark antibody, and a nanobody. In some preferred embodiments, this disclosure provides a derivative of an antibody disclosed herein comprising a detectable label fixably attached thereto, optionally wherein the detectable label is selected from the group consisting of fluorescein, DyLight, Cy3, Cy5, FITC, HiLyte Fluor 555, HiLyte Fluor 647, 5-carboxy-2,7-dichlorofluorescein, 5-carboxyfluorescein, 5-FAM, hydroxy tryptamine, 5-hydroxy tryptamine (5-HAT), 6-carboxyfluorescein (6-FAM), FITC, 6-carboxy-1,4-dichloro-2′,7′-dichlorofluorescein (TET), 6-carboxy-1,4-dichloro-2′,4′,5′,7′-tetrachlorofluorescein (HEX), 6-carboxy-4′,5′-dichloro-2′,7′-dimethoxyfluorescein (6-JOE), an Alexa fluor, Alexa fluor 350, Alexa fluor 405, Alexa fluor 430, Alexa fluor 488, Alexa fluor 500, Alexa fluor 514, Alexa fluor 532, Alexa fluor 546, Alexa fluor 555, Alexa fluor 568, Alexa fluor 594, Alexa fluor 610, Alexa fluor 633, Alexa fluor 635, Alexa fluor 647, Alexa fluor 660, Alexa fluor 680, Alexa fluor 700, Alexa fluor 750, a BODIPY fluorophores, BODIPY 492/515, BODIPY 493/503, BODIPY 500/510, BODIPY 505/515, BODIPY 530/550, BODIPY 542/563, BODIPY 558/568, BODIPY 564/570, BODIPY 576/589, BODIPY 581/591, BODIPY 630/650-X, BODIPY 650/665-X, BODIPY 665/676, FL, FL ATP, FI-Ceramide, R6G SE, TMR, TMR-X conjugate, TMR-X, SE, TR, TR ATP, TR-X SE, a rhodamine, rhodamine 110, rhodamine 123, rhodamine B, rhodamine B 200, rhodamine BB, rhodamine BG, rhodamine B extra, 5-carboxytetramethylrhodamine (5-TAMRA), 5 GLD, 6-carboxyrhodamine 6G, Lissamine, Lissamine Rhodamine B, Phallicidine, Phalloidine, rhodamine red, Rhod-2, 6-carboxy-X-rhodamine (ROX), carboxy-X-rhodamine (5-ROX), Sulphorhodamine B can C, Sulphorhodamine G Extra, 6-carboxytetramethylrhodamine (TAMRA), tetramethylrhodamine (TRITC), rhodamine WT, Texas Red, and Texas Red-X. In some preferred embodiments, this disclosure provides a derivative of an antibody disclosed herein comprising an effector moiety attached thereto, optionally wherein the effector moiety is selected from the group consisting of a cytotoxic drug, toxin, diphtheria A chain, exotoxin A chain, ricin A chain, abrin A chain, curcin, crotin, phenomycin, enomycin, and radiochemical, optionally comprising a cleavable linker positioned between the antibody and the effector moiety, wherein said cleavable linker releases the effector moiety into or within a cell. In some preferred embodiments, this disclosure provides an isolated polynucleotide encoding an antibody of this disclosure, optionally wherein a nucleic acid sequence of at least one of SEQ ID NOS. 63-84, as well as an expression vector and host cells comprising the same. In some preferred embodiments, this disclosure provides a composition comprising at least one antibody or derivative of disclosed herein; at least one isolated polynucleotide encoding such an antibody or derivative; or at least one expression vector comprising such a polynucleotides; and/or, at least one host cell comprising such a polynucleotide and/or expression vector; or a combination thereof; and, a pharmaceutically acceptable carrier.

In some preferred embodiments, this disclosure provides methods for detecting HER2 on a cell, the method comprising contacting a test biological sample with an antibody or derivative of this disclosure, combination thereof, or a composition (preferably a pharmaceutical composition) including any of the same and detecting the antibody (or antibodies) bound to the biological sample or components thereof. In some embodiments, the methods can include comparing the amount of binding to the test biological sample or components thereof to the amount of binding to a control biological sample or components thereof, wherein increased binding to the test biological sample or components thereof relative to the control biological sample or components thereof indicates the presence of a cell expressing HER2 in the test biological sample (e.g., wherein the test biological sample is a mammalian cell, tissue, or blood). The method may be in vivo method or an in vitro method.

In some preferred embodiments, this disclosure provides methods for treating, preventing and/or ameliorating cancer in a mammal by administering to the mammal at least one effective dose of a pharmaceutical composition comprising an antibody, derivative, and/or combination of this disclosure. In some embodiments, this disclosure provides such an antibody comprising a cytotoxic effector moiety attached thereto, optionally wherein the effector moiety is selected from the group consisting of a cytotoxic drug, toxin, diphtheria A chain, exotoxin A chain, ricin A chain, abrin A chain, curcin, crotin, phenomycin, enomycin, and radiochemical. In some embodiments, such antibodies can comprise a cleavable linker positioned between the antibody and the effector moiety, wherein said cleavable linker releases the effector moiety into or within a cell. In some such embodiments, the antibody is administered as an antibody-drug conjugate. In some embodiments, multiple doses are administered to the animal; and/or, the antibody is administered in a dosage amount of about 1 to 50 mg/kg.

In some embodiments, this disclosure provides a kit for detecting the expression of HER2 in or on a cell, the kit including an antibody or derivative of this disclosure and instructions for use. In some embodiments, the antibody or derivative can be in lyophilized form.

Other embodiments are also provided by this disclosure, as would be understood by those of ordinary skill in the art. All references cited within this disclosure are hereby incorporated by reference in their entirety. Certain embodiments are further described in the following examples. These embodiments are provided as examples only and are not intended to limit the scope of the claims in any way.

### EXAMPLES

The following are examples of antibodies described herein. It should be understood by those of ordinary skill in the art that the methods described herein are only exemplary as many other methods for doing the same are available to those persons.

### Example 1: Development of Fully Human Anti-HER2 Monoclonal Antibodies

Described herein are the methods used to produce fully human anti-HER2 monoclonal antibodies. The first group of fully human anti-HER2 antibodies are internalizing and competing with trastuzumab for binding to HER2. (6 antibodies); these antibodies can be used in vitro or in in vivo animal models for, e.g., neutralizing as well as internalizing properties. The second group of fully human anti-HER2 antibodies are internalizing and do not compete with trastuzumab for binding to HER2 receptor (5 antibodies); these antibodies can be used in vitro or in in vivo animal models for their internalizing properties. To produce the fully human antibodies described herein, three humanized mice (TC-mAb™) were immunized with the human recombinant His-tagged HER2 extracellular domain. The characteristics of these mice are published (Moriwaki, T., Abe, S., Oshimura, M. & Kazuki, Y. 2020, Transchromosomic technology for genomically humanized animals. Exp. Cell Res. 390, 111914.)

Human Ab producing TC mice (TC-mAb mice) stably maintain a mouse-derived engineered chromosome containing the entire human Ig heavy and kappa chain loci in a mouse Ig knockout background. Trans-chromosomic (Tc) mice carrying mini-chromosomes with human immunoglobulin (lg) loci can contribute to the development of fully human therapeutic monoclonal antibodies (Abs) when immunized with antigen of interest. In this case, TC-mAb mice were immunized with human recombinant His-tagged HER2 extracellular domain according to a schedule that has been previously validated. After 30 days, titer of sera from immunized mice was checked by EIA with human his-tagged HER2 ECD immobilized to Nickel plates followed by incubation with HRP-conjugated goat anti-human Fc secondary antibody. Mouse with the highest anti-HER2 titer was used to collect spleen and Lymph node B cells that were fused by electroporation to mouse myeloma HL-1 cells. Fused Hybridomas were single cell plated in semi-solid hybridoma culture Clonal Cell™ HY medium D (StemCell Technology) in 10 cm tissue culture plates. After 11 days, 1152 single hybridoma clones were picked from the semi-solid medium plates and transferred to 96 well dishes (one clone per well) in hybridoma culture medium E (StemCell Technology). After 4 days, culture media of the hybridoma clones were assayed by HER2-ECD EIA as described above. At initial screen, the top 313 clones with an OD>2.0 were selected. The clones were transferred to 48 well plates for confirmatory screening by HER2-ECD EIA as described above. 207 clones were confirmed strong positive. These clones were transferred to 6 well plates in duplicate in medium E. Cells were cryo-preserved in appropriate culture conditions for long-term storage in liquid nitrogen while culture media containing secreted antibodies were collected and stored for future evaluation and selection of hybridoma of interest. Antibodies produced by the hybridoma clones were examined for: 1) competition with Trastuzumab for binding to HER2-ECD by competition EIA; 2) internalization on HER2 overexpressing cells such as SKBR3 and AU565 cells; 3) inhibition of cellular signaling in HER2 overexpressing breast cancer cells SKBR3; 4) inhibition of migration of HER2 overexpressing breast cancer cells SKBR3; and, 5) growth inhibition of HER2 overexpressing cells SKBR3 cells by incubation with anti-HER2 antibodies in the presence of Fab-αHFc-NC-DM1. Fab-αHFc-NC-DM1 is a Fab fragment of an anti-human IgG Fc specific antibody conjugated to maytansinoid DM1 with a non-cleavable linker. The antibody portion is a polyclonal antibody which is specific to the Fc region of human IgGs. DM1 is a cytotoxic small molecule which inhibits cell division by blocking the polymerization of tubulin. The non-cleavable linker connecting DM1 to the antibody is stable in extracellular fluid but can be cleaved by unspecified mechanisms upon entering cells. Alternatively, the killing assay has been done with anti-HER2 antibodies directly conjugated via a linker to DM1. As shown in Example 2 below, hybridoma clones selected for these activities were further characterized by nucleotide and amino acid sequencing, and CDR determination.

1. Competition with Trastuzumab for Binding to HER2-ECD by EIA

Rationale: Trastuzumab is the FDA approved anti-Her2 therapeutic antibody that is currently used in the standard of care, either as a naked antibody or as an Antibody drug conjugate. Therefore, one of the screening assays and selection criteria were to examine the ability of the fully human HER2 antibodies to compete with trastuzumab for binding to HER2 protein.

Method: 96 well high binding EIA plates were coated with 10 ng of HER2 protein overnight at 4 C. After washing and blocking the plates with 4% milk proteins for 1 hour at 37 C, the plates were washed 3 times and then incubated for an hour at 37 C with either hybridoma supernatants or fully human purified antibodies from the 202 clones selected by confirmatory EIA. Trastuzumab was used as positive control and human IgG was used as negative control. After 1 hour incubation at 37 C, the plates were washed 6 times then incubated for 1 hour at 37 C with biotin labeled anti-HER2 antibody (anti-HER2 TRAST-biotin), followed by washing and addition of Streptavidin conjugated to Horseradish peroxidase (HRP). After washing, colorimetric substrate 3,3′,5,5′-tetramethylbenzidine (TMB) was added and absorbance at 620 nm was determined after 10 minutes. Results examined the ability of anti-Fully human antibodies to inhibit biding of anti-HER2 trastuzumab conjugated to biotin (anti-HER2TRAST-Biotin) to bind to the coated HER2.

Results: out of the 207 hybridoma clones tested, 27 hybridomas produced antibodies able to compete with anti-HER2 TRAST-biotin.

2. Internalization in Comparison to Trastuzumab Using pHab Probe

Rationale: Trastuzumab is an internalizing antibody. Upon binding to HER2 on the surface of HER2 expressing cells, the complex antibody-HER2 receptor gets internalized inside the cells in a temperature dependent manner. Based on this characteristic, antibody drug conjugate where trastuzumab is conjugated to a cytotoxic payload via a specific linker can deliver a cytotoxic drug to the targeted cells leading to their killing. Two ADCs using trastuzumab have been developed: T-DM1 (Kadcyla) and Trastuzumab conjugated to deruxtecan (Enhertu), which have both have been approved by the United States Food and Drug Administration (FDA). Thus, it was of interest to examine whether there are internalizing fully human anti-HER2 antibodies among those produced by the 207 selected hybridomas.

Method: Goat anti-human IgG (H+L) secondary antibody was conjugated to pH probe (Promega) following the manufacturer instruction manual. Anti-HER2 antibodies at 5 ug/ml were premixed for 30 minutes at 22-25 C with pHab labeled goat anti human antibody at 10 ug/ml (antibody combination). SKBR3 or AU565 cells were detached with 5 mM EDTA and 2-4×10⁵-cells were incubated with the antibody combination and placed in a 5% CO2 incubator for 20 hours. At the end of the incubation, cells were washed twice with cold PBS and resuspended in PBS and fluorescence was measured with flow intellycite as Mean FL2-H channel. If antibody internalizes the fluorescence unit value determined will increase.

Results: Using a cut off value of 10,000 RFU (relative fluorescence unit), we identified 96 hybridoma clones producing internalizing antibodies out of the 207 clones tested. The 27 clones that produced antibodies competing with Trastuzumab were all internalizing antibodies. The 27 internalizing antibodies were subsequently examined for their biological activities including inhibition of HER2 signaling in comparison to trastuzumab, further selection by inhibition of migration assay. Selected antibodies were then examined by killing assay.

3. Inhibition of p-AKT Phosphorylation (Inhibition of Cellular Signaling in HER2 Overexpressing Breast Cancer Cells SKBR3)

Rationale: It is known that in HER2 overexpressing cells such as breast cancer cells SKBR3, HR2 is phosphorylated leading to activation of AKT signaling pathways without affecting ERK1/2 pathway. Thus antibodies that block HER2 function would inhibit phosphorylation of AKT (P-AKT). Trastuzumab (anti-HER2 antibody) is known to block such phosphorylation. Thus, examining the ability of the selected antibodies to inhibit P-AKT is a reasonable approach for further selection of antibodies.

Method: SKBR3 cells which overexpress HER2 were used for these studies. Eight (8)×10⁴ cells were plated in 35 m dishes in 2 ml of McCoy-5A medium supplemented with 10% Fetal bovine serum. After 72 hours, the cells were washed twice with serum-free medium, serum starved for 2 hours before adding the antibodies to be tested at 10 μg/ml and collecting the cells with RIPA buffer in the presence of protease and phosphatase inhibitors. Cell lysates were subjected to SDS-Polyacrylamide gel electrophoresis followed by transfer to pdf membrane in order to perform western blot analysis with phospho-AKT and phospho-ERK1/2. Test antibodies were added with human IgG used as negative control and anti-Her2 antibody trastuzumab used as positive control.

Results: Antibodies able to inhibit P-HER2, P-ERK1/2 and/or P-AKT expression were further selected.

### 4. Inhibition of Migration by HER2 Overexpressing Breast Cancer Cells SKBR3

Rationale: HER2 overexpressing cells have a higher potential for migrating to distance sites. Thus, examining the ability of anti-HER2 antibodies to inhibit the ability of cells to migrate is a reasonable approach to examine and select efficacious anti-HER2 antibodies.

Method: Migration was measured using the Transwell method where cells are plated on top of a collagen coated 8 μm filter through which cells can migrate using the method described in Guha et al, 2020. Antibodies were added at increasing concentrations from 0 to 50 ug/ml with human IgG used as negative control and anti-HER2 antibody trastuzumab as positive control.

Results: Antibodies inhibited migration in a dose-dependent fashion with 50% inhibition at 50 μg/ml.

### 5. Stimulation of Cytotoxicity or Inhibition of Cell Proliferation in the Presence of Fab Anti-Human Fc Conjugated to DM1 Cytotoxic Payload

Rationale: Internalizing antibodies can deliver cytotoxic payload into cells having the target against which the internalizing antibodies have been developed. Measuring the killing efficacy of selected antibodies is a way to determine their internalizing capacity and their ability to deliver cytotoxic payload. Method: The ability of the selected antibodies to deliver to SKBR3 cells the cytotoxic payload DM-1 conjugated to a goat anti-human FAB was examined using method described by (Marquez, J., J. Dong, C. Dong, C. Tian and G. Serrero (2021). “Identification of Prostaglandin F2 Receptor Negative Regulator (PTGFRN) as an internalizable target in cancer cells for antibody-drug conjugate development.” PLOS One 16 (1): e0246197. Briefly 8000 SKBR3 cells were plated in a 96 wells plate in culture medium supplemented with anti-HER2 antibodies along with Fab-αHFc-NC-DM1 at various concentrations. Human IgG with Fab-αHFc-NC-DM1 was used as negative control. After 72 hours, the cell growth and cell killing was determined by using the Cell-Glo assay (Promega) according to the manufacturer instructions.

Results: The results regarding antibodies shown to stimulate cell killing are presented below.

Overall Results and Antibody Selection: Based on the five assays described above, six (6) hybridomas that produced antibodies that displayed the highest activity in internalization, and ability to compete with trastuzumab for binding to HER2 protein were selected. These six selected antibodies are 11D7, 2A2, 12E3, 2D7, 3G7, 8C1.

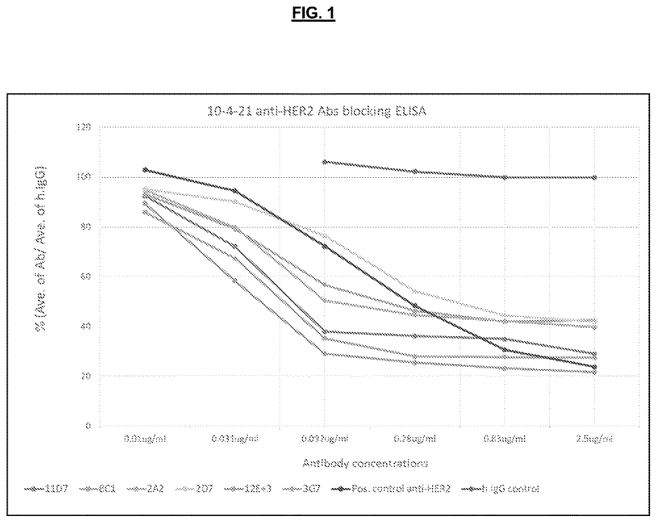

FIG. 1 illustrates the results of the competition assay of binding of trastuzumab to HER2 protein by increasing doses of the selected fully human anti-HER2 antibodies 11D7, 2A2, 12E3, 2D7, 3G7, 8C1 using the method described above. Trastuzumab and human IgG were used as positive and negative controls. FIG. 1 shows the results of a competition assay by the 6 selected fully human antibodies for trastuzumab binding to HER2 protein at concentrations from 0.01 μg/ml to 2.5 ug/ml. Five (5) out of six (6) antibodies had an ED 50. Five (5) out of the six (6) antibodies showed a strong inhibition of trastuzumab-biotin binding to HER2 with ED50 from 0.04 ug/ml for 12E3, 0.06 ug/ml for 11D7 and 3G7 and 0.09 ug/ml and 0.1 ug/ml for 2A2 and 8C1, with 2D7 having a similar ED50 as trastuzumab, respectively.

Antibodies 11D7, 12E3, 2D7, and 3G7 were determined to be of isotype IgG1. Antibodies 2A2 and 8C1 were to be of isotype IgG2a.

The affinity of the 11D7, 2A2, 12E3, 2D7, 3G7, 8C1, along with trastuzumab, for binding to HER2 was examined by Biolayer Interferometry using Octet Red 96 as shown in Table 6. Each of antibodies 11D7, 2A2, 12E3, 2D7, 3G7, 8C1 were found to have excellent Kd ranging up to 3.6×10⁻⁹M suitable for drug development. This is within the range of Kd measured for commercially available trastuzumab which is having a Kd of 3.1×10⁻¹⁰M in these experiments and the reported manufactured trastuzumab reported to have a Kd of 5×10⁻⁹M.

TABLE 6

| | Conc. | | | | | |
|---|---|---|---|---|---|---|
| Antibody | (nM) | KD (M) | kon(1/Ms) | kdis(1/s) | Full X\hat{ }2 | Full R\hat{ }2 |
| 2A2 | 50 | 3.82E−12 | 2.85E+05 | 1.09E−06 | 0.1453 | 0.9757 |
| 3G7 | 50 | 2.66E−10 | 2.54E+05 | 6.78E−05 | 0.1184 | 0.9825 |
| 12E3 | 50 | 2.67E−10 | 3.13E+05 | 8.38E−05 | 0.2183 | 0.9723 |
| 11D7 | 50 | 3.42E−10 | 3.06E+05 | 1.04E−04 | 0.2184 | 0.9772 |
| 8C1 | 50 | 4.64E−10 | 3.08E+05 | 1.43E−04 | 0.1446 | 0.9743 |
| 2D7 | 50 | 3.56E−09 | 1.31E+05 | 4.67E−04 | 0.0407 | 0.9878 |
| huIgG | 50 | NA | NA | NA | NA | NA |
| Anti- | 50 | 3.10E−10 | 2.38E+05 | 7.40E−05 | 0.0839 | 0.9864 |
| HER2 | | | | | | |
| TRAST | | | | | | |

A migration assay which measures the ability of cells to migrate through a Transwell coated with collagen is a well-recognized assay to examine one hallmark of tumorigenic potential and metastasis of cancer cells. Examination whether an antibody can inhibit migration can provide a measure of its future therapeutic potential and help in selection of antibodies to further its progression into pre-clinical studies. The method used has been described previously (Guha, R., B. Yue, J. Dong, A. Banerjee and G. Serrero (2021). “Anti-progranulin/GP88 antibody AG01 inhibits triple negative breast cancer cell proliferation and migration.” Breast Cancer Res Treat 186 (3): 637-653) except that the cells used are the HER2 overexpressing breast cancer SKBR3 cells and the time of migration is 40-48 hours with 75,000 cells in medium supplemented with 2% fetal bovine serum. The 6 selected antibodies were examined for their ability to inhibit migration which is a hallmark of tumorigenicity and metastasis. Table 7 summarizes the data of all assays carried out with the 11D7, 2A2, 12E3, 2D7, 3G7, and 8C1 antibodies, showing inhibition of migration of the SKBR3 cells.

The selected 11D7, 2A2, 12E3, 2D7, 3G7, and 8C1 fully human anti-HER2 antibodies have ability to induce cell killing of SKBR3 cells which overexpress HER2 when compared to trastuzumab, as shown in Table 7A. These data confirm that the 11D7, 2A2, 12E3, 2D7, 3G7, and 8C1 fully human anti-HER2 antibodies are internalizing and that they can deliver a cytotoxic payload and as such are well suited for being the development of antibody-drug conjugate (ADC).

TABLE 7A

| | | | | Killing | | |
|---|---|---|---|---|---|---|
| | Competition | | Western | assay at | | |
| | Trsatuzumab | | blot- | 0.3 ug/ml- | | |
| | binding to | Internalization: | inhibition of | Ab (2 nM), | Migration | |
| | HER2 at 0.1 | Fluorescent | P-AKT in | DMI(6 nM), | assay, at | |
| Clone# | ug/ml | Units | MCF-7wt | 72 hr | 50 μg/ml | Kd (M) |
| 2A2 | 50% | 10,890 | Yes | 55% | 38% | 3.82 × 10⁻¹² |
| 2D7 | 24% | 4500 | Yes | 50% | 59% | 3.56 × 10⁻⁹ |
| 3G7 | 65% | 14,000 | Yes | 67% | 24% | 2.66 × 10⁻¹⁰ |
| 8C1 | 43% | 6700 | Yes | 59% | 32% | 4.64 × 10⁻¹⁰ |
| 11D7 | 62% | 52,000 | Yes | 57% | 32% | 3.06 × 10⁻¹⁰ |
| 12E3 | 71% | 9000 | Yes | 67% | 45% | 2.67 × 10⁻¹⁰ |
| Commercial | 50% | 13,787 | No | 50% | 25% | 3.1 × 10⁻¹⁰ |
| anti-Her-2 | | | | | | |

The 12E3 was directly conjugated to DM1 using Mal-VC-PAB-DM1 linker payload component (MEDCHEM express) and following the manufacturer instructions. Table 7B shows that the 12E3 direct conjugate can inhibit cell proliferation at concentrations as low as 0.01 nM

TABLE 7B
Inhibition of SKBR3 proliferation
by 12E3-DM1 antibody drug conjugate

| 12E3-DM1 (nM) | 0 | 0.01 | 0.1 | 0.5 | 1 | 2.5 | 5 | 10 | 30 |
|---|---|---|---|---|---|---|---|---|---|
| % Control | 100 | 64 | 20 | 18 | 15 | 14 | 14 | 14 | 13 |

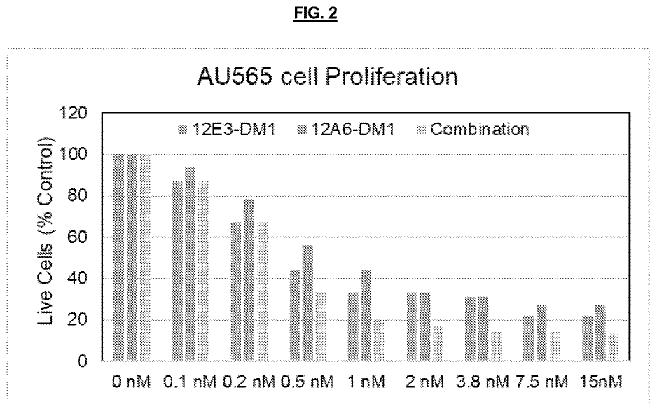

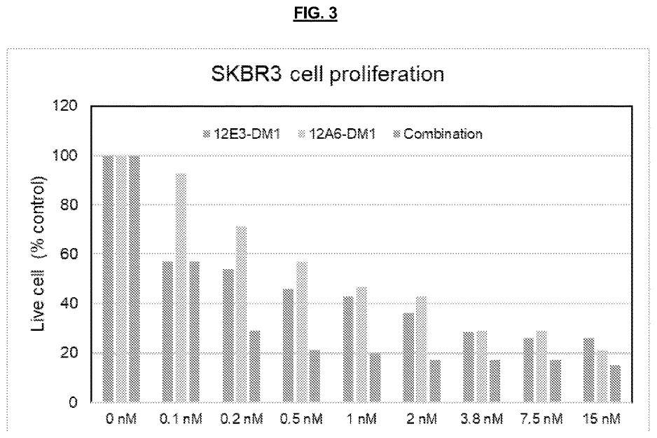

In another set of experiments, six mAbs clones from group A (competing with trastuzumab for binding to Her2) and five clones from group B (not competing with trastuzumab for binding to HER2) were selected. Of these, the biochemical and biological properties of group A 12E3 and group B 12A6 were investigated. 12E3 and 12A6 were each separately conjugated with DM1 to form the ADCs 12E3-DM1 and 12A6-DM1, respectively. These ADCs were examined for their ability to inhibit proliferation of HER2 overexpressing breast cancer cells in vitro alone or in combination on the HER2 overexpressing cell lines AU565 and SKBR3. As shown in FIG. 2, both conjugates, either alone or in combination reduced proliferation of AU565 cells in a dose-dependent fashion. As shown in FIG. 3, both conjugates, either alone or in combination reduced proliferation of SKBR3 cells in a dose-dependent fashion. Thus, this data shows that antibodies of group A (e.g., 12E3-DM1) and group B (e.g., 12A6-DM1) anti-HER2 ADCs inhibit HER2⁺ cell proliferation in a dose dependent fashion. The combination of ADCs from each group had a potentiating effect to inhibit cell proliferation showing advantage of combining anti-HER2 Mabs targeting different HER2 epitopes.

### Example 4: Sequence Analysis of Fully Human Antibodies 11D7, 2A2, 12E3, 2D7, 3G7, and 8C1

The hybridoma cell line was thawed and put in culture. The cells were counted and reverse transcription was performed to converted the RNA to cDNA. Several PCRs were performed to amplify the heavy chain and light chain variable region sequences with several pairs of mouse heavy and light chain specific primers. TA few of PCR products were loaded on an agarose gel to run the electrophoresis and check the PCR bands. The PCR products were mixed and sequenced by Miseq sequencer. The NGS sequencing data was processed with BCR analysis software. DNA sequence data from all constructs are analyzed and consensus sequences for heavy and light chain are determined. The consensus sequences are compared to known variable region sequences to rule out artifacts and/or process contamination. Consensus sequences were then analyzed using an online tool to verify that the sequences could encode a productive immunoglobulin (e.g., comprising heavy and light chains, etc.). After determining the amino acid sequences of such antibodies, the amino acid sequences of the CDRs of the antibodies were determined according to the Kabat method. The amino acid and polynucleotide sequences of the antibodies are shown below.

### 1. Fully Human 11D7 Antibody

AGRD017-11D7-IgG (Variable Heavy Chain (VH))

Amino Acid Sequence (CDRs underlined):
(SEQ ID NO: 41)
EVQLVESGGGLVQPGRSLRLSCAASGFTFDDYAMHWVRQAPGKGL
EWVSGIAWNGGILGYADSVKGRFTISRDNAKNSLYLQMNSLRAED
TALYYCAKDTGSYSHWYFDLWGRGTLVTVSS
DNA Sequence:
(SEQ ID NO: 63)
GAAGTGCAGCTGGTGGAGTCTGGGGGAGGCTTGGTACAGCCTGGC
AGGTCCCTGAGACTCTCCTGTGCAGCCTCTGGATTCACCTTTGAT
GATTATGCCATGCACTGGGTCCGGCAAGCTCCAGGGAAGGGCCTG
GAGTGGGTCTCAGGTATTGCTTGGAATGGTGGTATCTTAGGCTAT
GCGGACTCTGTGAAGGGCCGATTCACCATCTCCAGAGACAACGCC
AAGAACTCCCTGTATCTGCAAATGAACAGTCTGAGAGCTGAGGAC
ACGGCCTTGTATTACTGTGCAAAAGATACGGGGAGTTATTCTCAC
TGGTACTTCGATCTCTGGGGCCGTGGCACCCTGGTCACTGTCTCC
TCAG

| Region | Sequence Fragment | Residues | Length |
|---|---|---|---|
| HER1 | EVQLVESGGGLVQPGRSLRLSC | 1-30 | 30 |
| | AASGETED (SEQ ID NO: 85) | | |
| CDR-H1 | DYAMH (SEQ ID NO: 1) | 31-35 | 5 |
| HER2 | WVRQAPGKGLEWVS | 36-49 | 14 |
| | (SEQ ID NO: 86) | | |
| CDR-H2 | GIAWNGGILGYADSVKG | 50-66 | 17 |
| | (SEQ ID NO: 2) | | |
| HER3 | RFTISRDNAKNSLYLQM | 67-98 | 32 |
| | NSLRAEDTALYYCAK | | |
| | (SEQ ID NO: 87) | | |
| CDR-H3 | DIGSYSHWYFDL | 99-110 | 12 |
| | (SEQ ID NO: 3) | | |
| HER4 | WGRGTLVTVSS | 111-121 | 11 |
| | (SEQ ID NO: 88) | | |
| | | | 121 |

### AGRD017-11D7-IgK ((Variable Light Chain (VL))

Amino Acid Sequence (CDRs underlined):
(SEQ ID NO: 42)
EIVMTQSPATLSVSPGERATLSCRASQSVRSNLAWYQQKPGQAPR
LLIYGASTRATGIPARFSGSGSGTEFTLTISSLQSEDFAVYCCQQ
YNNWPTFGQGTKVEIK
DNA Sequence:
(SEQ ID NO: 64)
GAAATAGTGATGACGCAGTCTCCAGCCACCCTGTCTGTGTCTCCA
GGGGAAAGAGCCACCCTCTCCTGCAGGGCCAGTCAGAGTGTTAGG
AGCAACTTAGCCTGGTATCAGCAGAAACCTGGCCAGGCTCCCAGG
CTCCTCATCTATGGTGCATCCACCAGGGCCACTGGTATCCCAGCC
AGGTTCAGTGGCAGTGGGTCTGGGACAGAGTTCACTCTCACCATC
AGCAGCCTGCAGTCTGAAGATTTTGCAGTTTATTGCTGTCAGCAG
TATAATAACTGGCCTACGTTCGGCCAAGGGACCAAGGTGGAAATC
AAAC

| Region | Sequence Fragment | Residues | Length |
|---|---|---|---|
| LER1 | EIVMTQSPATLSVSPGERATL | 1-23 | 23 |
| | SC (SEQ ID NO: 89) | | |
| CDR-L1 | RASQSVRSNLA | 24-34 | 11 |
| | (SEQ ID NO: 4) | | |
| LER2 | WYQQKPGQAPRLLIY | 35-49 | 15 |
| | (SEQ ID NO: 90) | | |
| CDR-12 | GASTRAT (SEQ ID NO: 5) | 50-56 | 7 |
| LER3 | GIPARESGSGSGTEFTLTIS | 57-88 | 32 |
| | SLQSEDEAVYCC | | |
| | (SEQ ID NO: 91) | | |
| CDR-L3 | QQYNNWPT | 89-96 | 8 |
| | (SEQ ID NO: 6) | | |
| LFR4 | FGQGTKVEIK | 97-106 | 10 |
| | (SEQ ID NO: 92) | | |
| | | | 106 |

### 2. Fully Human 2A2 Antibody

### AGRD017-2A2-IgG ((Variable Heavy Chain (VH)

Amino Acid Sequence (CDRs underlined):
(SEQ ID NO: 43)
EVQLVESGGGLVQPGRSLRLSCAASGFTFDDYAMHWVRQAPGKGL
EWVSGISWNGGTLGYADSVKGRFTISRDNAKNSLYLQMNSLRAED
TALYYCAKDTGSYSHWYFDLWGRGTLVTVSS
DNA Sequence:
(SEQ ID NO: 65)
GAAGTGCAGTTGGTGGAGTCTGGGGGAGGCTTGGTACAGCCTGGC
AGGTCCCTGAGACTCTCCTGTGCAGCCTCTGGATTCACCTTTGAT
GATTATGCCATGCACTGGGTCCGGCAAGCTCCAGGGAAGGGCCTG
GAGTGGGTCTCAGGTATTAGTTGGAATGGTGGTACCTTAGGCTAT
GCGGACTCTGTGAAGGGCCGATTCACCATCTCCAGAGACAACGCC
AAGAACTCCCTGTATCTGCAAATGAACAGTCTGAGAGCTGAGGAC
ACGGCCTTGTATTACTGTGCAAAAGATACGGGGAGTTATTCTCAC
TGGTACTTCGATCTCTGGGGCCGTGGCACCCTGGTCACTGTCTCC
TCAG

| Region | Sequence Fragment | Residues | Length |
|---|---|---|---|
| HER1 | EVQLVESGGGLV | 1-30 | 30 |
| | QPGRSLRLSCA | | |
| | ASGFTED | | |
| | (SEQ ID NO: 93) | | |
| CDR-H1 | DYAMH | 31-35 | 5 |
| | (SEQ ID NO: 1) | | |
| HER2 | WVRQAPGKGLEWVS | 36-49 | 14 |
| | (SEQ ID NO: 94) | | |
| CDR-H2 | GISWNGGTLGYADSVKG | 50-66 | 17 |
| | (SEQ ID NO: 7) | | |
| HFR3 | RFTISRDNAKNSLYLQMN | 67-98 | 32 |
| | SLRAEDTALYYCAK | | |
| | (SEQ ID NO: 95) | | |
| CDR-H3 | DTGSYSHWYEDL | 99-110 | 12 |
| | (SEQ ID NO: 3) | | |
| HER 4 | WGRGTLVTVSS | 111-121 | 11 |
| | (SEQ ID NO: 96) | | |
| | | | 121 |

### AGRD017-2A2-IgK (Variable Light Chain (VL))

Amino Acid Sequence (CDRs underlined):
(SEQ ID NO: 44)
EIVMTQSPATLSVSPGERATLSCRASQSVRSNLAWYQQKPGQAPR
LLIYGASTRATGIPARESGSGSGTEFTLTISSLHSEDFAVYYCQQ
YNNWPTFGQGTKVEIK
DNA Sequence:
(SEQ ID NO: 66)
GAAATAGTGATGACGCAGTCTCCAGCCACCCIGTCTGTGTCTCCA
GGGGAAAGAGCCACCCTCTCCTGCAGGGCCAGTCAGAGIGTTAGG
AGCAACTTAGCCTGGTATCAGCAGAAACCTGGCCAGGCTCCCAGG
CTCCTCATCTATGGTGCATCCACCAGGGCCACTGGTATCCCAGCC
AGGTTCAGTGGCAGTGGGTCTGGGACAGAGTTCACTCTCACCATT
AGCAGCCTGCACTCTGAAGATTTTGCAGTTTATTACTGTCAGCAG
TATAACAACTGGCCTACGTTCGGCCAAGGGACCAAGGTGGAAATC
AAAC

| Region | Sequence Fragment | Residues | Length |
|---|---|---|---|
| LFR1 | EIVMTQSPATLSVSPGERATL | 1-23 | 23 |
| | SC (SEQ ID NO: 97) | | |
| CDR-L1 | RASQSVRSNLA | 24-34 | 11 |
| | (SEQ ID NO: 4) | | |
| LFR2 | WYQQKPGQAPRLLIY | 35-49 | 15 |
| | (SEQ ID NO: 98) | | |
| CDR-L2 | GASTRAT (SEQ ID NO: 5) | 50-56 | 7 |
| LFR3 | GIPARFSGSGSGTEFTLT | 57-88 | 32 |
| | ISSLHSEDFAVYYC | | |
| | (SEQ ID NO: 99) | | |
| CDR-L3 | QQYNNWPT (SEQ ID NO: 6) | 89-96 | 8 |
| LFR4 | FGQGTKVEIK | 97-106 | 10 |
| | (SEQ ID NO: 100) | | |
| | | | 106 |

### 2. Fully Human 12E3 Antibody

### AGRD017-12E3-IgG ((Variable Heavy Chain (VH))

Amino Acid Sequence (CDRs underlined):
(SEQ ID NO: 45)
EVQLVGSGGGLVQPGRSLRLSCAASGFTFDDYAMHWVRQAPGKGL
EWVSGISWNGGTLGYAGSVKGRFTISRDNAKNSLYLQMNSLRAED
TALYYCAKDTGSYSHWYFDLWGRGTLVTVSS
DNA Sequence:
(SEQ ID NO: 67)
GAAGTGCAGCTGGTGGGGTCTGGGGGAGGTTTGGTACAGCCTGGC
AGGTCCCTGAGACTCTCCTGTGCAGCCTCTGGATTCACCTTTGAT
GATTATGCCATGCACTGGGTCCGGCAAGCTCCAGGGAAGGGCCTG
GAGTGGGTCTCAGGTATTAGTTGGAATGGTGGTACGTTGGGCTAT
GCGGGCTCTGTGAAGGGCCGATTCACCATCTCCAGAGACAACGCC
AAGAACTCCCTGTATCTGCAAATGAACAGTCTGAGAGCTGAGGAC
ACGGCCTTGTATTACTGTGCAAAAGATACGGGGAGTTATTCTCAC
TGGTACTTCGATCTCTGGGGCCGTGGCACCCTGGTCACTGTCTCC
TCAG

| Region | Sequence Fragment | Residues | Length |
|---|---|---|---|
| HFR1 | EVQLVGSGGGLVQPGRSL | 1-30 | 30 |
| | RLSCAASGFTED | | |
| | (SEQ ID NO: 101) | | |
| CDR-H1 | DYAMH | 31-35 | 5 |
| | (SEQ ID NO: 1) | | |
| HFR2 | WVRQAPGKGLEWVS | 36-49 | 14 |
| | (SEQ ID NO: 102) | | |
| CDR-H2 | GISWNGGTLGYAGS | 50-66 | 17 |
| | VKG (SEQ ID NO: 9) | | |
| HFR3 | RFTISRDNAKNSLYLQ | 67-98 | 32 |
| | MNSLRAEDTALYYCAK | | |
| | (SEQ ID NO: 103) | | |
| CDR-H3 | DTGSYSHWYFDL | 99-110 | 12 |
| | (SEQ ID NO: 3) | | |
| HER4 | WGRGTLVTVSS | 111-121 | 11 |
| | (SEQ ID NO: 104) | | |
| | | | 121 |

### AGRD017-12E3-IgK (Variable Light Chain (VL))

Amino Acid Sequence (CDRs underlined):
(SEQ ID NO: 46)
EIVMTQSPATLSVSPGERATLSCRASQSVRSNLAWYQQKPGQAPR
LLIYGASTRATGIPARCSGSGSGTEFTLTISSLQSEDFAVYYCQQ
YNNWPTFGQGTKVEIK
DNA Sequence:
(SEQ ID NO: 68)
GAAATAGTGATGACGCAGTCTCCAGCCACCCTGTCTGTGTCTCCA
GGGGAAAGAGCCACCCTCTCCTGCAGGGCCAGTCAGAGTGTTAGA
AGCAACTTAGCCTGGTACCAGCAGAAACCTGGCCAGGCTCCCAGG
CTCCTCATCTATGGTGCATCCACCAGGGCCACTGGTATCCCAGCC
AGGTGCAGTGGCAGTGGGTCTGGGACAGAGTTCACTCTCACCATC
AGCAGCCTGCAGTCTGAGGATTTTGCAGTTTATTACTGTCAGCAG
TATAATAACTGGCCTACGTTCGGCCAAGGGACCAAGGTGGAAATC
AAAC

| Region | Sequence Fragment | Residues | Length |
|---|---|---|---|
| LFR1 | EIVMTQSPATLSVSPGE | 1-23 | 23 |
| | RATLSC | | |
| | (SEQ ID NO: 105) | | |
| CDR-L1 | RASQSVRSNLA | 24-34 | 11 |
| | (SEQ ID NO: 4) | | |
| LFR2 | WYQQKPGQAPRLLI | 35-49 | 15 |
| | Y (SEQ ID NO: 106) | | |
| CDR-L2 | GASTRAT | 50-56 | 7 |
| | (SEQ ID NO: 5) | | |
| LFR3 | GIPARCSGSGSGTEFTLT | 57-88 | 32 |
| | ISSLQSEDFAVYYC | | |
| | (SEQ ID NO: 107) | | |
| CDR-L3 | QQYNNWPT | 89-96 | 8 |
| | (SEQ ID NO: 6) | | |
| LFR4 | FGQGTKVEIK | 97-106 | 10 |
| | (SEQ ID NO: 108) | | |
| | | | 106 |

### 3. Fully Human 207 Antibody

### AGRD017-207-IgG (Variable Heavy Chain (VH))

Amino Acid Sequence (CDRs underlined):
(SEQ ID NO: 47)
EVQLVESGGGLVQPGRSLRLSCAASGFTEDDYAMHWVRQAPGKGL
EWVSGISWNSGTMGYAGSVKGRFTISRDNAKNSLYLQMNSLRAED
TALYYCAKDTGSYSHWYFDLWGRGTLVTVSS
DNA Sequence:
(SEQ ID NO: 69)
GAAGTGCAGCTGGTGGAGTCTGGGGGAGGTTTGGTACAGCCTGGC
AGGTCCCTGAGACTCTCCTGTGCAGCCTCTGGATTCACCTTTGAT
GATTATGCCATGCACTGGGTCCGGCAAGCTCCAGGGAAGGGCCTG
GAGTGGGTCTCAGGTATTAGTTGGAATAGTGGTACCATGGGCTAT
GCGGGCTCTGTGAAGGGCCGATTCACCATCTCCAGAGACAACGCC
AAGAACTCCCTGTATCTGCAAATGAACAGTCTGAGAGCTGAGGAC
ACGGCCTTGTATTACTGTGCAAAAGATACGGGGAGTTATTCTCAC
TGGTACTTCGATCTCTGGGGCCGTGGCACCCTGGTCACTGTCTCC
TCAG

| Region | Sequence Fragment | Residues | Length |
|---|---|---|---|
| HFR1 | EVQLVESGGGLVQPGR | 1-30 | 30 |
| | SLRLSCAASGFTED | | |
| | (SEQ ID NO: 109) | | |
| CDR-H1 | DYAMH | 31-35 | 5 |
| | (SEQ ID NO: 1) | | |
| HFR2 | WVRQAPGKGLEWVS | 36-49 | 14 |
| | (SEQ ID NO: 110) | | |
| CDR-H2 | GISWNSGTMGYAGSV | 50-66 | 17 |
| | KG (SEQ ID NO: 10) | | |
| HFR3 | RFTISRDNAKNSLYLQ | 67-98 | 32 |
| | MNSLRAEDTALYYCAK | | |
| | (SEQ ID NO: 111) | | |
| CDR-H3 | DTGSYSHWYFDL | 99-110 | 12 |
| | (SEQ ID NO: 3) | | |
| HFR4 | WGRGTLVTVSS | 111-121 | 11 |
| | (SEQ ID NO: 112) | | |
| | | | 121 |

### AGRD017-2D7-IgK ((Variable Light Chain (VL))

Amino Acid Sequence (CDRs underlined):
(SEQ ID NO: 48)
EIVMTQSPATLSVSPGERATLSCRASQSVRTNLAWYQQKPGQAPR
LLIYGASTRATGIPARFSGSGSGTEFTLTISSLQSEDFAVYYCQQ
YNNWPTFGQGTKVEIK
DNA Sequence:
(SEQ ID NO: 70)
GAAATAGTGATGACGCAGTCTCCAGCCACCCTGTCTGTGTCTCCA
GGGGAAAGAGCCACCCTCTCCTGCAGGGCCAGTCAGAGTGTTAGG
ACCAACTTAGCCTGGTACCAGCAGAAACCTGGCCAGGCTCCCAGG
CTCCTCATCTATGGTGCATCCACCAGGGCCACTGGCATCCCAGCC
AGGTTCAGTGGTAGTGGGTCTGGGACAGAGTTCACTCTCACCATC
AGCAGCCTGCAGTCTGAAGATTTTGCAGTTTATTACTGTCAGCAG
TATAATAACTGGCCTACGTTCGGCCAAGGGACCAAGGTGGAAATC
AAAC

| Region | Sequence Fragment | Residues | Length |
|---|---|---|---|
| LFR1 | EIVMTQSPATLSVSPG | 1-23 | 23 |
| | ERATLSC | | |
| | (SEQ ID NO: 113) | | |
| CDR-L1 | RASQSVRTNLA | 24-34 | 11 |
| | (SEQ ID NO: 11) | | |
| LFR2 | WYQQKPGQAPRLLIY | 35-49 | 15 |
| | (SEQ ID NO: 114) | | |
| CDR-L2 | GASTRAT | 50-56 | 7 |
| | (SEQ ID NO: 5) | | |
| LFR3 | GIPARFSGSGSGTEFTL | 57-88 | 32 |
| | TISSLQSEDFAVYYC | | |
| | (SEQ ID NO: 115) | | |
| CDR-L3 | QQYNNWPT | 89-96 | 8 |
| | (SEQ ID NO: 6) | | |
| LFR4 | FGQGTKVEIK | 97-106 | 10 |
| | (SEQ ID NO: 116) | | |
| | | | 106 |

### 4. Fully Human 3G7 Antibody

### AGRD017-3G7-IgG ((Variable Heavy Chain (VH))

Amino Acid Sequence (CDRs underlined):
(SEQ ID NO: 49)
EVQLVESGGGLVQPGRSLRLSCAASGFTEDDYAMHWVRQAPGKGL
EWVSGVSWNGGTMGYAGSVKGRFTISRDNAKNSLYLQMNSLRAED
TALYYCAKDTGSYSHWYFDLWGRGTLVTVSS
DNA Sequence:
(SEQ ID NO: 71)
GAAGTGCAGCTGGTGGAGTCTGGGGGAGGTTTGGTACAGCCTGGC
AGGTCCCTGAGACTCTCCTGTGCAGCCTCTGGATTCACCTTTGAT
GATTATGCCATGCACTGGGTCCGGCAAGCTCCAGGGAAGGGCCTG
GAGTGGGTCTCAGGTGTTAGTTGGAATGGTGGTACCATGGGCTAT
GCGGGCTCTGTGAAGGGCCGATTCACCATCTCCAGAGACAACGCC
AAGAACTCCCTGTATCTGCAAATGAACAGTCTGAGAGCTGAGGAC
ACGGCCTTGTATTACTGTGCAAAAGATACGGGGAGTTATTCTCAC
TGGTACTTCGATCTCTGGGGCCGTGGCACCCTGGTCACTGTCTCC
TCAG

| Region | Sequence Fragment | Residues | Length |
|---|---|---|---|
| HFR1 | EVQLVESGGGLVQPGRS | 1-30 | 30 |
| | LRLSCAASGFTFD (SEQ | | |
| | ID NO: 117) | | |
| CDR-H1 | DYAMH | 31-35 | 5 |
| | (SEQ ID NO: 1) | | |
| HER2 | WVRQAPGKGLEWVS | 36-49 | 14 |
| | (SEQ ID NO: 118) | | |
| CDR-H2 | GVSWNGGTMGYAGSVKG | 50-66 | 17 |
| | (SEQ ID NO: 12) | | |
| HFR3 | RFTISRDNAKNSLYLQMN | 67-98 | 32 |
| | SLRAEDTALYYCAK | | |
| | (SEQ ID NO: 119) | | |
| CDR-H3 | DTGSYSHWYFDL | 99-110 | 12 |
| | (SEQ ID NO: 3) | | |
| HFR4 | WGRGTLVTVSS | 111-121 | 11 |
| | (SEQ ID NO: 120) | | |
| | | | 121 |

### AGRD017-3G7-IgK (Variable Light Chain (VL))

Amino Acid Sequence (CDRs underlined):
(SEQ ID NO: 50)
EIVMTQSPATLSVSPGERATLSCRASQSVRSNLAWYQQKPGQAPR
LLIYGASTRATGIPGRFSGSGSGTEFILTISSLQSEDFAVYYCQQ
YNNWPTFGQGTKVEIK
DNA Sequence:
(SEQ ID NO: 72)
GAAATAGTGATGACGCAGTCTCCAGCCACCCTGTCTGTGTCTCCA
GGGGAAAGAGCCACCCTCTCCTGCAGGGCCAGTCAGAGTGTTAGG
AGCAACTTAGCCTGGTACCAGCAGAAACCTGGCCAGGCTCCCAGG
CTCCTCATCTATGGTGCATCCACCAGGGCCACTGGTATCCCAGGC
AGGTTCAGTGGCAGTGGGTCTGGGACAGAGTTCATTCTCACCATC
AGCAGCCTGCAGTCTGAAGATTTTGCAGTTTATTACTGTCAGCAG
TATAATAACTGGCCTACGTTCGGCCAAGGGACCAAGGTGGAAATC
AAAC

| Region | Sequence Fragment | Residues | Length |
|---|---|---|---|
| LFR1 | EIVMTQSPATLSVSPGE | 1-23 | 23 |
| | RATLSC (SEQ ID NO: | | |
| | 121) | | |
| CDR-L1 | RASQSVRSNLA | 24-34 | 11 |
| | (SEQ ID NO: 4) | | |
| LFR2 | WYQQKPGQAPRLLIY | 35-49 | 15 |
| | (SEQ ID NO: 122) | | |
| CDR-L2 | GASTRAT | 50-56 | 7 |
| | (SEQ ID NO: 5) | | |
| LFR3 | GIPGRFSGSGSGTEFILT | 57-88 | 32 |
| | ISSLQSEDFAVYYC | | |
| | (SEQ ID NO: 123) | | |
| CDR-L3 | QQYNNWPT | 89-96 | 8 |
| | (SEQ ID NO: 6) | | |
| LFR4 | FGQGTKVEIK | 97-106 | 10 |
| | (SEQ ID NO: 124) | | |
| | | | 106 |

### 5. Fully Human 8C1 Antibody

### AGRD017-8C1-IgG (Variable Heavy Chain (VH)):

Amino Acid Sequence (CDRs underlined):
(SEQ ID NO: 51)
EVQLVESGGGLVQPGRSLRLSCTASGFTEDDYAMHWVRQAPGKGL
EWVSGISWNGGTLGYADSVKGRFTISRDNAKNSLYLQMNSLRAED
TALYYCAKDTGSYSHWYFDLWGRGTLVTVSS
DNA Sequence:
(SEQ ID NO: 73)
GAAGTTGCAGTTGGTGGAGTCTGGGGGAGGCTTGGTACAGCCTGG
CAGGTCCCTGAGACTCTCCTGTACAGCCTCTGGATTCACCTTTGA
TGATTATGCCATGCACTGGGTCCGGCAAGCTCCAGGGAAGGGCCT
GGAGTGGGTCTCAGGTATTAGTTGGAATGGTGGTACCTTAGGCTA
TGCGGACTCTGTGAAGGGCCGATTCACCATCTCCAGAGACAACGC
CAAGAACTCCCTGTATCTGCAAATGAACAGTCTGAGAGCTGAGGA
CACGGCCTTGTATTACTGTGCAAAAGATACGGGGAGTTATTCTCA
CTGGTACTTCGATCTCTGGGGCCGTGGCACCCTGGTCACTGTCTC
CTCAG

| Region | Sequence Fragment | Residues | Length |
|---|---|---|---|
| HFR1 | EVQLVESGGGLVQPGRS | 1-30 | 30 |
| | LRLSCTASGFTED | | |
| | (SEQ ID NO: 125) | | |
| CDR-H1 | DYAMH | 31-35 | 5 |
| | (SEQ ID NO: 1) | | |
| HFR2 | WVRQAPGKGLEWVS | 36-49 | 14 |
| | (SEQ ID NO: 126) | | |
| CDR-H2 | GISWNGGTLGYADSVKG | 50-66 | 17 |
| | (SEQ ID NO: 13) | | |
| HFR3 | RFTISRDNAKNSLYLQ | 67-98 | 32 |
| | MNSLRAEDTALYYCAK | | |
| | (SEQ ID NO: 127) | | |
| CDR-H3 | DTGSYSHWYFDL | 99-110 | 12 |
| | (SEQ ID NO: 3) | | |
| HER4 | WGRGTLVTVSS | 111-121 | 11 |
| | (SEQ ID NO: 128) | | |
| | | | 121 |

### AGRD017-8C1-IgK (Variable Light Chain (VL)):

Amino Acid Sequence:
(SEQ ID NO: 52)
EIVMTQSPATLSVSPGERATLSCRASQSVRSNLAWYQQKPGQAPR
LLIYGASTRATGIPARESGSGSGTEFTLTISSLASEDFAVYYCQQ
YNNWPTFGQGTKVEIK
DNA Sequence:
(SEQ ID NO: 74)
GAAATAGTGATGACGCAGTCTCCAGCCACCCTGTCTGTGTCTCCA
GGGGAAAGAGCCACCCTCTCCTGTAGGGCCAGTCAGAGTGTTAGG
AGCAACTTAGCCTGGTATCAGCAGAAACCTGGCCAGGCTCCCAGG
CTCCTCATCTATGGTGCATCCACCAGGGCCACTGGTATCCCAGCC
AGGTTCAGTGGCAGTGGGTCTGGGACAGAGTTCACTCTCACCATT
AGCAGCCTGCACTCTGAAGATTTTGCAGTTTATTACTGTCAGCAG
TATAACAACTGGCCTACGTTCGGCCAAGGGACCAAGGTGGAAATC
AAAC

| Region | Sequence Fragment | Residues | Length |
|---|---|---|---|
| LFR1 | EIVMTQSPATLSVSPGE | 1-23 | 23 |
| | RATLSC | | |
| | (SEQ ID NO: 129) | | |
| CDR-L1 | RASQSVRSNLA | 24-34 | 11 |
| | (SEQ ID NO: 4) | | |
| LFR2 | WYQQKPGQAPRLLIY | 35-49 | 15 |
| | (SEQ ID NO: 130) | | |
| CDR-L2 | GASTRAT | 50-56 | 7 |
| | (SEQ ID NO: 5) | | |
| LFR3 | GIPARFSGSGSGTEF | 57-88 | 32 |
| | TLTISSLHSEDFAVYYC | | |
| | (SEQ ID NO: 131) | | |
| CDR-L3 | QQYNNWPT | 89-96 | 8 |
| | (SEQ ID NO: 6) | | |
| LFR4 | FGQGTKVEIK | 97-106 | 10 |
| | (SEQ ID NO: 132) | | |
| | | | 106 |

In order to perform an alignment of the 11D7, 2A2, 12E3, 2D7, 3G7, and 8C1 amino acid sequences with trastuzumab and pertuzumab, the sequences for both antibodies were obtained from the published reports and are shown below:

### Trastuzumab AA Sequence

Anti-HER2 Light chain (1 and 2)
(SEQ ID NO: 133)
DIQMTQSPSSLSASVGDRVTITCRASQDVNTAVAWYQQKPGKAPK
LLIYSASFLYSGVPSRESGSRSGTDFTLTISSLQPEDFATYYCQQ
HYTTPPTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVTKSENRGEC
Anti-HER2 Heavy chain (1 and 2)
(SEQ ID NO: 134)
EVQLVESGGGLVQPGGSLRLSCAASGENIKDTYIHWVRQAPGKGL
EWVARIYPTNGYTRYADSVKGRFTISADTSKNTAYLQMNSLRAED
TAVYYCSRWGGDGFYAMDYWGQGTLVTVSSASTKGPSVFPLAPSS
KSTSGGTAALGCLVKDYFPEPVTVSWNSGALTSGVHTFPAVLQSS
GLYSLSSVVTVPSSSLGTQTYICNVNHKPSNTKVDKKVEPKSCDK
THTCPPCPAPELLGGPSVFLFPPKPKDTLMISRTPEVTCVVVDVS
HEDPEVKENWYVDGVEVHNAKTKPREEQYNSTYRVVSVLTVLHQD
WLNGKEYKCKVSNKALPAPIEKTISKAKGQPREPQVYTLPPSREE
MTKNQVSLTCLVKGFYPSDIAVEWESNGQPENNYKTTPPVLDSDG
SFFLYSKLTVDKSRWQQGNVFSCSVMHEALHNHYTQKSLSLSPGK
Pertuzumab AA sequence
Amino acid sequence for pertuzumab light chain
(SEQ ID NO: 135)
DIQMTQSPSSLSASVGDRVTITCKASQDVSIGVAWYQQKPGKAPK
LLIYSASYRYTGVPSRESGSGSGTDFTLTISSLQPEDFATYYCQQ
YYIYPYTFGQGTKVEIKRTVAAPSVFIFPPSDEQLKSGTASVVCL
LNNFYPREAKVQWKVDNALQSGNSQESVTEQDSKDSTYSLSSTLT
LSKADYEKHKVYACEVTHQGLSSPVIKSENRGEC
Amino acid sequence for pertuzumab heavy chain
(SEQ ID NO: 136)
EVQLVESGGGLVQPGGSLRLSCAASGFTFTDYTMDWVRQAPGKGL
EWVADVNPNSGGSIYNQRFKGRFTLSVDRSKNTLYLQMNSLRAED
TAVYYCARNLGPSFYFDYWGQGTLVTVSSASTKGPSVEPLAPSSK
STSGGTAALGCLVKDYFPEPVTVSWNSGALTSGVHTFPAVLQSSG
LYSLSSVVTVPSSSLGTQTYICNVNHKPSNTKVDKKVEPKSCDKT
HTCPPCPAPELLGGPSVFLFPPKPKDTIMISRTPEVTCVVVDVSH
EDPEVKENWYVDGVEVHNAKTKPREEQYNSTYRVVSVLTVLHQDW
LNGKEYKCKVSNKALPAPIEKTISKAKGQPREPQVYTLPPSREEM
TKNQVSLTCLVKGFYPSDIAVEWESNGQPENNYKTTPPVLDSDGS
FFLYSKLTVDKSRWQQGNVFSCSVMHEALHNHYTQKSLSLSPG

The amino acid sequences of the variable regions of the 11D7, 2A2, 12E3, 2D7, 3G7, and 8C1 fully human antibodies were aligned to trastuzumab and pertuzumab with CDR determination according to Kabat, with the CDR1, CDR2, and CDR3 of each underlined.

Heavy chain alignment

| | |
|---|---|
| | CDR1 CDR2 |
| 12E3 | EVQLVGSGGGLVQPGRSIRLSCAASGFTFDDYAMHWVRQAPGKGLEWVSGISWNGGTLGY 60 |
| 11D7 | EVQLVESGGGLVQPGRSIRLSCAASGFTFDDYAMHWVRQAPGKGLEWVSGIAWNGGILGY 60 |
| 8C1 | EVQLVESGGGLVQPGRSLRLSCTASGFTFDDYAMHWVRQAPGKGLEWVSGISWNGGILGY 60 |
| 2A2 | EVQLVESGGGLVQPGRSLRLSCAASGFTFDDYAMHWVRQAPGKGLEWVSGISWNGGTLGY 60 |
| 2D7 | EVQLVESGGGLVQPGRSLRLSCAASGFTFDDYAMHWVRQAPGKGLEWVSGISWNSGTMGY 60 |
| 3G | EVQLVESGGGLVQPGRSLRLSCAASGFTFDDYAMHWVRQAPGKGLEWVSGVSWNGGTMGY 60 |
| Trastuza | EVQLVESGGGLVQPGGSLRLSCAASGFNIKDTYIHWVRQAPGKGLEWVARIYPINGYTRY 60 |
| Pertuzu | EVQLVESGGGLVQPGGSLRLSCAASGFTFTDYTMDWVRQAPGKGLEWVADVNPNSGGSIY 60 |
| | *** *********** ******:****.: * :.*************: : ..* * |
| | CDR3 |
| 12E3 | AGSVKGRFTISRDNAKNSLYLQMNSLRAEDTALYYCAKDTGSYSHWYFDLWGRGTLVTVS 120 |
| 11D7 | ADSVKGRFTISRDNAKNSLYLQMNSLRAEDTALYYCAKDTGSYSHWYFDLWGRGTLVTVS 120 |
| 801 | ADSVKGRFTISRDNAKNSLYLQMNSLRAEDTALYYCAKDTGSYSHWYFDLNGRGTLVTVS 120 |
| 2A2 | ADSVKGRFTISRDNAKNSLYLQMNSLRAEDTALYYCAKDTGSYSHWYFDLWGRGTLVTVS 120 |
| 207 | AGSVKGRFTISRDNAKNSLYLQMNSLRAEDTALYYCAKDTGSYSHWYFDLWGRGTLVTVS 120 |
| 3G7 | AGSVKGRFTISRDNAKNSLYLQMNSLRAEDTALYYCAKDTGSYSHWYFDLWGRGTLVTVS 120 |
| Trastuzu | ADSVKGRFTISADISKNTYYLQMNSLRAEDTAVYYCSRWGGD-GFYAMDYWGQGTLVTVS 119 |
| Pertuzu | NQRFKGRFTLSVDRSKNTLYLQMNSLRAEDTAVYYCARNLGP-SFY-FDYWGQGILVTVS 118 |
| | .*****:* * :**: *************:***:: * ..: :* **:******* |
| 12E3 | S 121 |
| 11D7 | S 121 |
| 801 | S 121 |
| 2A2 | S 121 |
| 207 | S 121 |
| 3G7 | S 121 |
| Trastuzu | S 121 |
| Pertuzu | S 119 |
| | * |

From these alignments, it is clear that the CDR3 regions (CDRs underlined) of each variable heavy chain (VH) of 11D7, 2A2, 12E3, 2D7, 3G7, and 8C1 are different from those of trastuzumab (“Trastuzu”) and pertuzumab (“Pertuzu”).

Light chain alignment

| | |
|---|---|
| | CDR1 CDR2 |
| 8C1 | EIVMTQSPATLSVSPGERATLSCRASQSVRSNLAWYQQKPGQAPRLLIYGASTRATGIPA 60 |
| 2A2 | EIVMTQSPATLSVSPGERATLSCRASQSVRSNLAWYQQKPGQAPRLLIYGASTRATGIPA 60 |
| 11D7 | EIVMTQSPATLSVSPGERATLSCRASQSVRSNLAWYQQKPGQAPRLLIYGASTRATGIPA 60 |
| 12E3 | EIVMTQSPATLSVSPGERATLSCRASQSVRSNLAWYQQKPGQAPRLLIYGASTRATGIPA 60 |
| 2D7 | EIVMIQSPATLSVSPGERATLSCRASQSVRINLAWYQQKPGQAPRLLIYGASTRATGIPA 60 |
| 367 | EIVMTQSPATLSVSPGEPATLSCRASQSVRSNLAWYQQXPGQAPRLLIYGASTRATGIPG 60 |
| Trastuzu | DIQMTQSPSSLSASVGDRVTITCRASQDVNTAVAWYQQKPGKAPKLLIYSASFLYSGVPS 60 |
| Pertuzu | DIQMTQSPSSLSASVGDRVTITCKASQDVSIGVAWYQQKPGKAPKLLIYSASYRYTGVPS 60 |
| | :* *****::**.* *:*.*::*:***.* :********:**:****.** :*:*. |
| | CDR3 |
| 8C1 | RFSGSGSGTEFTLTISSLASEDFAVYYCQQYNNW-FTFGQGTKVEIK 106 |
| 2A2 | RFSGSGSGTEFTLTISSLHSEDFAVYYCQQYNNW-PTFGQGIKVEIK 106 |
| 11D7 | RFSGSGSGTEFTLTISSLQSEDFAVYCQQQYNNW-PTFGQGIKVEIK 106 |
| 12E3 | RCSGSGSGTEFELTISSIQSEDFAVYYCQQYNNW-PTFGQGTKVEIK 106 |
| 2D7 | RFSGSGSGTEFTLTISSLQSEDEAVYYCQQYNNW-PTFGQGIKVEIK 106 |
| 3G7 | RFSGSGSGTEFILTISSIQSEDFAVYYCQQYNNW-PTFGQGTKVEIK 106 |
| Trastuzu | RFSGSRSGTDFTLTISSLQPEDFATYYCQQHYTTPPTFGQGTKVEIK 107 |
| Pertuzu | RFSGSGSGTDFTLTISSLQPEDFATYYCQQYYTYPYTFGQGTKVEIK 107 |
| | * *** ***:* ******: ****.* ***: *********** |

From these alignments, it is clear that the CDR3 regions of each variable light chain (VH) of 11D7, 2A2, 12E3, 2D7, 3G7, and 8C1 are different from those of trastuzumab and pertuzumab.

AGRD017 6 clones Alignment, H chain

| | |
|---|---|
| 12E3 | EVQLVGSGGGLVQPGRSLRLSCAASGFTFDDYAMHWVRQAPGKGLEWVSGISWNGGTLGY 60 |
| 11D7 | EVQLVESGGGLVQPGRSERLSCAASGETFDDYAMAWVRQAPGKGLEWYSGIAWNGGILGY 60 |
| 8C1 | EVQLVESGGGLVQPGRSLRLSCTASGFTEDDYAMHWYRQAPGKGLEWVSGISWNGGTIGY 60 |
| 2A2 | EVQLVESGGGLVQPGRSERLSCAASGETEDDCAMAWVRQAPGKGLEWVSGISWNGGTLGY 60 |
| 2D7 | EVQLVESGGGLVQPGRSLRISCAASGFIFDDYAMRWVRQAPGKGLEKVSGISWNSGTMGY 60 |
| 3G7 | EVQLVESGGGLVQPGRSERLSCAASGETEDDYAMHWVRQAPGKGLEWVSGVSWNGGINGY 60 |
| | ***** ****************:***************************::**.* :** |
| 12E3 | AGSVKGRETISRDNAKNSLYLQMNSLPAEDTALYYCAKDTGSYSHWYFDLWGRGTLVTVS 120 |
| 1137 | ADSVKGRFTISRDNAKNSLYLQMNSLRAEDTALYYCAKDTGSYSHWYFDLWGRSTLVTYS 120 |
| 8C1 | ADSVKGRETISRDNAKNSLYLQMNSLRAEDTALYYCAKDIGSYSHWYFDLWGRGTLVTVS 120 |
| 2A2 | ADSVKGRFTISRQNAKNSLYLQMNSLRAEDTALYYCAKDTGSYSAKYFDLWGRGTLVTYS 120 |
| 207 | AGSVKGRFTISRDNAKNSLYLQMNSLRAEDTALYYCAKDIGSYSHWYEDLWGRGTLVTVS 120 |
| 3G7 | AGSVRGRFTISRQNAKNSLYLQMNSLRAEDTALYYCAKDTGSYSHWYFDLWGRGTLVTVS 120 |
| 12E3 | S 121 |
| 1137 | S 121 |
| 8C1 | S 121 |
| 2A2 | S 121 |
| 207 | S 121 |
| 3G7 | S 121 |
| 12E3 | S 121 |
| | * |

AGRD017 6 clones Alignment, L chain

| | |
|---|---|
| 12E3 | EIVMTQSPATLSVSPGERATLSCRASQSVRSNLAWYQQKPGQAPRLLTYGASTRATGIPA 60 |
| 1137 | EIVMTQSPATLSVSPGERATLSCRASQSVRSNLAWYQQKPGQAPRILTYGASTRATGIPA 60 |
| 8C1 | EIVMTQSPATLSVSPGERATLSCRASQSVRSNLAWYQQKPGQAPRILTYGASTRATGIPA 60 |
| 2A2 | EIVMTQSPATLSVSPGERATLSCRASQSVRSNLAWYQQKPGQAPRILTYGASTRATGIPA 60 |
| 207 | EIVMTQSPATLSVSPGERATLSCRASQSVRINLAWYQQKPGQAPRILIYGASTRATGIPA 60 |
| 3G7 | EIVMTQSPATLSVSPGERATLSCRASQSVRSNLAWYQQKEGQAPRILTYGASTRATGIPG 60 |
| | ******************************:****************************. |
| 8C1 | RFSGSGSGTEFTLTISSLHSEDFAVYYCQQYNNWPTFGQGTKVEIK 106 |
| 2A2 | RFSGSGSGTEFTLTISSLHSEDFAVYYCQQYNNWPTFGQGTKVEIK 106 |
| 11D7 | RFSGSGSGTEFTLTISSLQSEDFAVYCCQQYNNWPTFGQGTKVEIK 106 |
| 12E3 | RCSGSGSGTEFELTISSIQSEDFAVYYCQQYNNWPTFGQGTKVEIK 106 |
| 2D7 | RFSGSGSGTEFTLTISSLQSEDFAVYYCQQYNNWPTFGQGTKVEIK 106 |
| 3G7 | RFSGSGSGTEFILTISSLQSEDFAVYYCQQYNNWPTFGQGTKVEIK 106 |
| | * ********* ******:******* ******************* |

It can be concluded from these studies that the 11D7, 2A2, 12E3, 2D7, 3G7, and 8C1 AGRD017 clones have similar sequences but are distinct antibodies from Trastuzumab and Pertuzumab.

### Example 5: Anti-HER-2 Fully Human Antibodies that do not Compete with Trastuzumab for Binding to HER-2 Receptor

Efforts were also focused at characterizing anti-HER2 fully human antibodies that bound to HER-2 receptor, were internalizing but did not compete with trastuzumab for binding to HER-2. The rationale for that is to select fully human antibodies directed at HER2 alternate epitope from the ones that are targeted with present commercially available antibodies such as pertuzumab and trastuzumab and have therapeutic applications as being internalizing antibodies that can be used for antibody-drug conjugates. These antibodies were developed by the same antibody development procedure described above using human HER-ECD as immunogen. Antibody selection was performed by: 1) ELISA binding to HER-ECD; 2) Flow binding to SKBR3 cells; 3) No competition with trastuzumab for binding to HER2; 4) Internalization; 6) Antibody Kd determination and epitope binning; 7) cytotoxicity (killing) assay by anti-HER2 selected antibodies in presence of Fab-αHFc-NC-DM1 as described above; and, 8) IgG Sequencing. Among the antibodies studied with these characteristics, especially internalization and lack of competitive binding with trastuzumab for binding to HER-2, five (5) antibodies were selected (2A1, 7F9, 9E4, 11C9, and 12A6; all being of isotype IgG1-kappa (IgG1K)). It was also found through epitope binning studies that 2A1, 7F9, 9E4, 11C9, and 12A6 bind to epitopes on HER2 that are distinct from Trastuzumab. Table 8 summarizes the functional properties of these antibodies (“Clone[s]).

TABLE 8

| | | Her-2 | | ADC | | |
|---|---|---|---|---|---|---|
| | *Flow | binding | **Internal- | Killing | | |
| Clone | Binding | competition | ization | effect | Kd (M) | |
| 2A1 | 175% | 0 | 121% | 80% | 5.34 | 10−9 |
| 7F9 | 180% | 0 | 157% | 86% | 3.82 | 10−10 |
| 9E4 | 362% | 0 | 277% | 83% | 1.09 | 10−8 |
| 11C9 | 200% | 0 | 187% | 86% | 1.92 | 10−8 |
| 12A6 | 270 | 0 | 100% | 82% | 9.8 | 10−9 |

*flow binding data were reported as % of trastuzumab binding used as positive control and expressed as 100%.
**internalization data were reported as % of the internalization value of trastuzumab used as positive control and expressed as 100%.
All experiments were carried out using SKBR3 cells.

The amino acid and nucleic acid sequences of the variable heavy (VH) and variable light (VL) polypeptide chains of the fully human 2A1, 7F9, 9E4, 11C9, and 12A6 antibodies were determined as described above. The CDRs were determined according to the Kabat method and are highlighted in the amino acid sequences shown below.

1. 2A1 antibody

AGRD017-2A1-IgG (VH)
Amino Acid Sequence:
(SEQ ID NO: 53)
EVQLVESGGGLVQPGGSLRLSCAASGFTFSSYWMSWVRQAPGKGL
EWVANIKQDGSEKYYVDSVKGRFTISRDNAKNSLYLQMNSLRAED
TAVYYCAREVGPGDYWGQGTLVTVSS
DNA Sequence:
(SEQ ID NO: 75)
GAGGTGCAGCTGGTGGAGTCTGGGGGAGGCTTGGTCCAGCCTGGG
GGGTCCCTGAGACTCTCCTGTGCAGCCTCTGGATTCACCTTTAGT
AGCTATTGGATGAGCTGGGTCCGCCAGGCTCCAGGGAAGGGGCTG
GAGTGGGTGGCCAACATAAAGCAAGATGGAAGTGAGAAATACTAT
GTGGACTCTGTGAAGGGCCGATTCACCATCTCCAGAGACAACGCC
AAGAACTCACTGTATCTGCAAATGAACAGCCTGAGAGCCGAGGAC
ACGGCTGTGTATTACTGTGCGAGAGAGGTGGGGCCCGGGGACTAC
TGGGGCCAGGGAACCCTGGTCACCGTCTCCTCAG

| Region | Sequence Fragment | Residues | Length |
|---|---|---|---|
| HFR1 | EVQLVESGGGLVQPGGS | 1-30 | 30 |
| | LRLSCAASGFTFS | | |
| | (SEQ ID NO: 137) | | |
| CDR-H1 | SYWMS | 31-35 | 5 |
| | (SEQ ID NO: 14) | | |
| HFR2 | WVRQAPGKGLEWVA | 36-49 | 14 |
| | (SEQ ID NO: 138) | | |
| CDR-H2 | NIKQDGSEKYYVDSVKG | 50-66 | 17 |
| | (SEQ ID NO: 15) | | |
| HFR3 | RFTISRDNAKNSLYLQ | 67-98 | 32 |
| | MNSLRAEDTAVYYCAR | | |
| | (SEQ ID NO: 139) | | |
| CDR-H3 | EVGPGDY | 99-105 | 7 |
| | (SEQ ID NO: 16) | | |
| HFR4 | WGQGTLVTVSS | 106-116 | 11 |
| | (SEQ ID NO: 140) | | |
| | | | 116 |

116

AGRD017-2A1-IgK (VL)
Amino Acid Sequence:
(SEQ ID NO: 54)
DIQMTQSPSTLSASVGDRVTITCRASQSISNWVAWYQQKPGKAPK
LLIHTASSLESGVPSRESGSGSGTEFTLTISSLQPDDFATYYCQQ
YYSYSPTFGQGTKVEIK
DNA Sequence:
(SEQ ID NO: 76)
GACATCCAGATGACCCAGTCTCCTTCCACCCTGTCTGCATCTGTA
GGAGACAGAGTCACCATCACTTGCCGGGCCAGTCAGAGTATTAGT
AACTGGGGGCCTGGTATCAGCAGAAACCAGGGAAAGCCCCTAAGC
TCCTGATCCATACGGCATCTAGCTTAGAAAGTGGGGTCCCATCAA
GGTTCAGCGGCAGTGGATCTGGGACAGAATTCACTCTCACCATCA
GCAGCCTGCAGCCTGATGATTTTGCAACTTATTACTGCCAACAGT
ATTATAGTTATTCTCCGACGTTCGGCCAAGGGACCAAGGTGGAAA
TCAAAC

| Region | Sequence Fragment | Residues | Length |
|---|---|---|---|
| LFR1 | DIQMTQSPSTLSASV | 1-23 | 23 |
| | GDRVTITC | | |
| | (SEQ ID NO: 141) | | |
| CDR-L1 | RASQSISNWVA | 24-34 | 11 |
| | (SEQ ID NO: 17) | | |
| LFR2 | WYQQKPGKAPKLLIH | 35-49 | 15 |
| | (SEQ ID NO: 142) | | |
| CDR-L2 | TASSLES | 50-56 | 7 |
| | (SEQ ID NO: 18) | | |
| LFR3 | GVPSRFSGSGSGTEFTL | 57-88 | 32 |
| | TISSLQPDDFATYYC | | |
| | (SEQ ID NO: 143) | | |
| CDR-L3 | QQYYSYSPT | 89-97 | 9 |
| | (SEQ ID NO: 19) | | |
| LFR4 | FGQGTKVEIK | 98-107 | 10 |
| | (SEQ ID NO: 144) | | |
| | | | 107 |

### 2. ZE9 Antibody

AGRD017-7F9-IgG (VH)
Amino Acid Sequence:
(SEQ ID NO: 55)
QMQLVQSGPEVKKPGTSVKVSCKASGFTFTSSAVQWVRQARGQRL
EWIGWIVVGSGNTNYAQKFQERVTITRDMSTSTAYMELSSLRSED
TAVYYCAAGYYGSGSPYYYYYGMDVWGQGTTVTVSS
DNA Sequence:
(SEQ ID NO: 77)
CAAATGCAGCTGGTGCAGTCTGGGCCTGAGGTGAAGAAGCCTGGG
ACCTCAGTGAAGGTCTCCTGCAAGGCTTCTGGATTCACCTTTACT
AGCTCTGCTGTGCAGTGGGTGCGACAGGCTCGTGGACAACGCCTT
GAGTGGATAGGATGGATCGTCGTTGGCAGTGGTAACACAAACTAC
GCACAGAAGTTCCAGGAAAGAGTCACCATTACCAGGGACATGTCC
ACAAGCACAGCCTACATGGAGCTGAGCAGCCTGAGATCCGAGGAC
ACGGCCGTGTATTACTGTGCGGCGGGGTACTATGGTTCGGGGAGT
CCTTACTACTACTACTACGGTATGGACGTCTGGGGCCAAGGGACC
ACGGTCACCGTCTCCTCAG

| Region | Sequence Fragment | Residues | Length |
|---|---|---|---|
| HFR1 | QMQLVQSGPEVKKPGTS | 1-30 | 30 |
| | VKVSCKASGFTFT | | |
| | (SEQ ID NO: 145) | | |
| CDR-H1 | SSAVQ | 31-35 | 5 |
| | (SEQ ID NO: 20) | | |
| HFR2 | WVRQARGQRLEWIG | 36-49 | 14 |
| | (SEQ ID NO: 146) | | |
| CDR-H2 | WIVVGSGNTNYAQKFQE | 50-66 | 17 |
| | (SEQ ID NO: 21) | | |
| HER3 | RVTITRDMSTSTAYMELS | 67-98 | 32 |
| | SLRSEDTAVYYCAA | | |
| | (SEQ ID NO: 147) | | |
| CDR-H3 | GYYGSGSPYYYYYGMDV | 99-115 | 17 |
| | (SEQ ID NO: 22) | | |
| HFR4 | WGQGTTVTVSS | 116-126 | 11 |
| | (SEQ ID NO: 148) | | |
| | | | 126 |

AGRD017-ZE9-IgK (VL)
Amino Acid Sequence:
(SEQ ID NO: 56)
DIQMTQSPSSVSASVGDRVTITCRASQGISSWLAWYQQKSGKAPK
LLIYAASSLQSGVPSRFSGSGSGTDFTLTINSLQPEDFATYYCQQ
VDSFPFTEGGGTKVESK
DNA Sequence:
(SEQ ID NO: 78)
GACATCCAGATGACCCAGTCTCCATCTTCCGTGTCTGCATCTGTA
GGAGACAGAGTCACCATCACTTGTCGGGCGAGTCAGGGTATTAGC
AGCTGGTTAGCCTGGTATCAGCAGAAATCAGGGAAAGCCCCTAAG
CTCCTGATCTATGCTGCATCTAGTTTGCAAAGTGGGGTCCCATCA
AGGTTCAGCGGCAGTGGATCTGGGACAGATTTCACTCTCACCATC
AACAGCCTGCAGCCTGAAGATTTTGCAACTTACTATTGTCAACAG
GTTGACAGTTTCCCGTTCACTTTCGGCGGAGGGACCAAGGTGGAG
AGCAAAC

| Region | Sequence Fragment | Residues | Length |
|---|---|---|---|
| LFR1 | DIQMTQSPSSVSASVGDRV | 1-23 | 23 |
| | TITC | | |
| | (SEQ ID NO: 149) | | |
| CDR-L1 | RASQGISSWLA | 24-34 | 11 |
| | (SEQ ID NO: 23) | | |
| LFR2 | WYQQKSGKAPKLLIY | 35-49 | 15 |
| | (SEQ ID NO: 150) | | |
| CDR-L2 | AASSLQS | 50-56 | 7 |
| | (SEQ ID NO: 24) | | |
| LFR3 | GVPSRFSGSGSGTDFTL | 57-88 | 32 |
| | TINSLQPEDFATYYC | | |
| | (SEQ ID NO: 151) | | |
| CDR-L3 | QQVDSFPFT | 89-97 | 9 |
| | (SEQ ID NO: 25) | | |
| LFR4 | FGGGTKVESK | 98-107 | 10 |
| | (SEQ ID NO: 152) | | |
| | | | 107 |

### 3. 9E4 Antibody

AGRD017-9E4-IgG (VH)
Amino Acid Sequence:
(SEQ ID NO: 57)
QVQLVESGGGLVKPGGSLRLSCAASGFTFSDYYMSWLRQAPGRGL
EWVSYISSSGITIYYADSVKGRFTISRDNAKNSLYLQMNSLRAED
TAVYYCARDGQQLADYYYYGMDVWGQGTTVTVSS
DNA Sequence:
(SEQ ID NO: 79)
CAGGTGCAGCTGGTGGAGTCTGGGGGAGGCTTGGTCAAGCCTGGA
GGGTCCCTGAGACTCTCCTGTGCAGCCTCTGGATTCACCTTCAGT
GACTACTACATGAGCIGGCTCCGCCAGGCTCCAGGGAGGGGGCTG
GAGTGGGTTTCATACATTAGTAGTAGTGGTATTACCATATACTAC
GCAGACTCTGTGAAGGGCCGATTCACCATCTCCAGGGACAACGCC
AAGAACTCACTGTATCTGCAAATGAACAGCCTGAGAGCCGAGGAC
ACGGCTGTGTATTACTGTGCGAGAGATGGACAGCAGCTGGCCGAC
TACTACTACTACGGTATGGACGTCTGGGGCCAAGGGACCACGGTC
ACCGTCTCCTCAG

| Region | Sequence Fragment | Residues | Length |
|---|---|---|---|
| HFR1 | QVQLVESGGGLVKPGGSL | 1-30 | 30 |
| | RLSCAASGFTFS | | |
| | (SEQ ID NO: 153) | | |
| CDR-H1 | DYYMS | 31-35 | 5 |
| | (SEQ ID NO: 26) | | |
| HFR2 | WLRQAPGRGLEWVS | 36-49 | 14 |
| | (SEQ ID NO: 154) | | |
| CDR-H2 | YISSSGITIYYADSVKG | 50-66 | 17 |
| | (SEQ ID NO: 27) | | |
| HFR3 | RFTISRDNAKNSLYLQ | 67-98 | 32 |
| | MNSLRAEDTAVYYCAR | | |
| | (SEQ ID NO: 155) | | |
| CDR-H3 | DGQQLADYYYYGMDV | 99-113 | 15 |
| | (SEQ ID NO: 28) | | |
| HFR4 | WGQGTTVTVSS | 114-124 | 11 |
| | (SEQ ID NO: 156) | | 124 |

AGRD017-9E4-lgK (VL)
Amino Acid Sequence:
(SEQ ID NO: 58)
EIVLTQSPGILSLSPGERATLSCRASQSVSSSYLTWYQQKPGQAP
RLLIYGASSRATGIPDRFSGSGSGTDFTLTISRLEPEDEAVYYCQ
QYGSSLTEGGGTKVEIK
DNA Sequence:
(SEQ ID NO: 80)
GAAATTGTGTTGACGCAGTCTCCAGGCACCCTGTCTTTGTCTCCA
GGGGAAAGAGCCACCCTCTCCTGCAGGGCCAGTCAGAGTGTTAGC
AGCAGCTACTTAACCTGGTACCAGCAGAAACCTGGCCAGGCTCCC
AGGCTCCTCATCTATGGTGCATCCAGCAGGGCCACTGGCATCCCA
GACAGGTTCAGTGGCAGTGGGTCTGGGACAGACTTCACTCTCACC
ATCAGCAGACTGGAGCCTGAAGATTTTGCAGTGTATTACTGTCAG
CAGTATGGTAGCTCACTCACTTTCGGCGGAGGGACCAAGGTGGAG
ATCAAAC

| Region | Sequence Fragment | Residues | Length |
|---|---|---|---|
| LFR1 | EIVLTQSPGTLSLSPGE | 1-23 | 23 |
| | RATLSC (SEQ ID NO: | | |
| | 157) | | |
| CDR-L1 | RASQSVSSSYLT | 24-35 | 12 |
| | (SEQ ID NO: 29) | | |
| LFR2 | WYQQKPGQAPRLLIY | 36-50 | 15 |
| | (SEQ ID NO: 158) | | |
| CDR-L2 | GASSRAT | 51-57 | 7 |
| | (SEQ ID NO: 30) | | |
| LFR3 | GIPDRESGSGSGTDFTLT | 58-89 | 32 |
| | ISRLEPEDFAVYYC | | |
| | (SEQ ID NO: 159) | | |
| CDR-L3 | QQYGSSLT | 90-97 | 8 |
| | (SEQ ID NO: 31) | | |
| LFR4 | FGGGTKVEIK | 98-107 | 10 |
| | (SEQ ID NO: 160) | | |
| | | | 107 |

### 4. 11C9 Antibody

AGRD017-11C9-IgG (VH)
Amino Acid Sequence:
(SEQ ID NO: 59)
QVQLVESGGGVVQPGRSLRLSCAASGFTFSSYGMHWVRQAPGKGL
EWVAVIWYDGGNKDYADSVKGRFTISRDNSKNTLYLQMNSLRAED
TAVYYCARVGQLAHYYYGMDVWGQGTTVTVSS
DNA Sequence:
(SEQ ID NO: 81)
CAGGTGCAGCTGGTGGAGTCTGGGGGAGGCGTGGTCCAGCCTGGG
AGGTCCCTGAGACTCTCCTGTGCAGCGTCTGGATTCACGTTCAGT
AGCTATGGCATGCACTGGGTCCGCCAGGCTCCAGGCAAGGGGCTG
GAGTGGGTGGCAGTTATATGGTATGATGGAGGTAATAAAGACTAT
GCAGACTCCGTGAAGGGCCGATTCACCATCTCCAGAGACAATTCC
AAGAACACGCTGTATCTGCAAATGAACAGCCTGAGAGCCGAGGAC
ACGGCTGTGTATTACTGTGCGAGAGTAGGGCAGCTGGCACACTAC
TACTACGGTATGGACGTCTGGGGCCAAGGGACCACGGTCACCGTC
TCCTCAG

| Region | Sequence Fragment | Residues | Length |
|---|---|---|---|
| HFR1 | QVQLVESGGGVVQPGRSL | 1-30 | 30 |
| | RLSCAASGFTFS | | |
| | (SEQ ID NO: 161) | | |
| CDR-H1 | SYGMH | 31-35 | 5 |
| | (SEQ ID NO: 32) | | |
| HFR2 | WVRQAPGKGLEWVA | 36-49 | 14 |
| | (SEQ ID NO: 162) | | |
| CDR-H2 | VIWYDGGNKDYADSVKG | 50-66 | 17 |
| | (SEQ ID NO: 33) | | |
| HFR3 | RFTISRDNSKNTLYLQM | 67-98 | 32 |
| | NSLRAEDTAVYYCAR | | |
| | (SEQ ID NO: 163) | | |
| CDR-H3 | VGQLAHYYYGMDV | 99-111 | 13 |
| | (SEQ ID NO: 34) | | |
| HFR4 | WGQGTTVTVSS | 112-122 | 11 |
| | (SEQ ID NO: 164) | | |
| | | | 122 |

AGRD017-11C9-IgK (VL)
Amino Acid Sequence:
(SEQ ID NO: 60)
DIVMTQSPLSLPVTPGEPASISCRSSQSLLHSNGYNYLDWYLQKP
GQSPQLLIYLGSNRASGVPDRFSGSGSGTDFTLKISRVEAEDVGV
YYCMQALQSPPTFGQGTKVEIK
DNA Sequence:
(SEQ ID NO: 82)
GATATTGTGATGACTCAGTCTCCACTCTCCCTGCCCGTCACCCCT
GGAGAGCCGGCCTCCATCTCCTGCAGGTCTAGTCAGAGCCTCCTG
CATAGTAATGGATACAACTATTTGGATTGGTACCTGCAGAAGCCA
GGGCAGTCTCCACAGCTCCTGATCTATTTGGGTTCTAATCGGGCC
TCCGGGGTCCCTGACAGGTTCAGTGGCAGTGGATCAGGCACAGAT
TTTACACTGAAAATCAGCAGAGTGGAGGCTGAGGATGTTGGGGTT
TATTACTGCATGCAAGCTCTACAAAGTCCTCCGACGTTCGGCCAA
GGGACCAAGGTGGAAATCAAAC

| Region | Sequence Fragment | Residues | Length |
|---|---|---|---|
| LFR1 | DIVMTQSPLSLPVTPGEPA | 1-23 | 23 |
| | SISC (SEQ ID NO: 165) | | |
| CDR-L1 | RSSQSLLHSNGYNYLD | 24-39 | 16 |
| | (SEQ ID NO: 35) | | |
| LFR2 | WYLQKPGQSPQLLIY | 40-54 | 15 |
| | (SEQ ID NO: 166) | | |
| CDR-L2 | ILGSNRAS | 55-61 | 7 |
| | (SEQ ID NO: 36) | | |
| LFR3 | GVPDRFSGSGSGTDFTLKIS | 62-93 | 32 |
| | RVEAEDVGVYYC (SEQ | | |
| | ID NO: 167) | | |
| CDR-L3 | MQALQSPPT | 94-102 | 9 |
| | (SEQ ID NO: 37) | | |
| LFR4 | FGQGTKVEIK | 103-112 | 10 |
| | (SEQ ID NO: 168) | | |
| | | | 112 |

### 5. 12A6 Antibody

AGRD017-12A6-IqG (VH)
Amino Acid Sequence:
(SEQ ID NO: 61)
QVQLVESGGGVVQPGRSLRLSCAASGFTFSSYGMHWVRQAPGKGL
EWVAVIWYDGSDDYYADSVKGRFTISRDNSKNTLYLQMNSLRAED
TAVYYCARELVRGASFDYWGQGTLVTVSS
DNA Sequence:
(SEQ ID NO: 83)
CAGGTGCAACTGGTGGAGTCTGGGGGAGGCGTGGTCCAGCCTGGG
AGGTCCCTGAGACTCTCCTGTGCAGCGTCTGGCTTCACCTTCAGT
AGCTATGGCATGCACTGGGTCCGCCAGGCTCCAGGCAAGGGGCTG
GAGTGGGTGGCAGTTATATGGTATGATGGAAGTGATGACTACTAT
GCAGACTCCGTGAAGGGCCGATTCACCATCTCCAGAGACAATTCC
AAGAACACGCTGTATCTGCAAATGAACAGCCTGAGAGCCGAGGAC
ACGGCTGTGTATTACTGTGCGAGAGAGCTGGTTCGGGGAGCCTCC
TTTGACTACTGGGGCCAGGGAACCCTGGTCACCGTCTCCTCAG

| Region | Sequence Fragment | Residues | Length |
|---|---|---|---|
| HFR1 | QVQLVESGGGVVQPGRS | 1-30 | 30 |
| | LRLSCAASGFTES | | |
| | (SEQ ID NO: 169) | | |
| CDR-H1 | SYGMH | 31-35 | 5 |
| | (SEQ ID NO: (32) | | |
| HFR2 | WVRQAPGKGLEWVA | 36-49 | 14 |
| | (SEQ ID NO: 170) | | |
| CDR-H2 | VIWYDGSDDYYADSVKG | 50-66 | 17 |
| | (SEQ ID NO: 38) | | |
| HFR3 | RFTISRDNSKNTLYLQMNS | 67-98 | 32 |
| | LRAEDTAVYYCAR (SEQ | | |
| | ID NO: 171) | | |
| CDR-H3 | ELVRGASFDY | 99-108 | 10 |
| | (SEQ ID NO: 39) | | |
| HFR4 | WGQGTLVTVSS | 109-119 | 11 |
| | (SEQ ID NO: 172) | | |
| | | | 119 |

### AGRD017-12A6-IgK (VL)

Amino Acid Sequence:
(SEQ ID NO: 62)
EILMTQSPLSLPVTPGEPATISCRSSQSLLHSNGYNYLDWYLQKP
GQSPQLLIYLGSNRASGVPDRESGSGSGTDFTLRISRVEABDVGV
YYCMQALQTPLTEGGGTKVEIK
DNA Sequence:
(SEQ ID NO: 84)
GAAATTTTGATGACTCAGTCTCCACTCTCCCTGCCCGTCACCCCT
GGAGAGCCGGCCACCATCTCCTGCAGGTCTAGTCAGAGCCTCCTC
CATAGTAATGGATACAACTATTTGGATTGGTACCTGCAGAAGCCA
GGGCAGTCTCCACAGCTCCTGATCTATTTGGGTTCTAATCGGGCC
TCCGGGGTCCCTGACAGGTTCAGTGGCAGTGGATCAGGCACAGAT
TTTACACTGAGAATCAGCAGAGTGGAGGCTGAGGATGTTGGGGTT
TATTACTGCATGCAAGCTCTACAAACTCCGCTCACTTTCGGCGGA
GGGACCAAGGTGGAGATCAAAC

| Region | Sequence Fragment | Residues | Length |
|---|---|---|---|
| LFR1 | EILMTQSPLSLPVTPGEPAT | 1-23 | 23 |
| | ISC (SEQ ID NO: 173) | | |
| CDR-L1 | RSSQSLLHSNGYNYLD | 24-39 | 16 |
| | (SEQ ID NO: 35) | | |
| LFR2 | WYLQKPGQSPQLLIY | 40-54 | 15 |
| | (SEQ ID NO: 174) | | |
| CDR-L2 | LGSNRAS | 55-61 | 7 |
| | (SEQ ID NO: 36) | | |
| LFR3 | GVPDRFSGSGSGTDFTLRI | 62-93 | 32 |
| | SRVEAEDVGVYYC | | |
| | (SEQ ID NO: 175) | | |
| CDR-L3 | MQALQTPLT | 94-102 | 9 |
| | (SEQ ID NO: 40) | | |
| LFR4 | FGGGTKVEIK | 103-112 | 10 |
| | (SEQ ID NO: 176) | | |
| | | | 112 |

The variable heavy (VH) and variable light (VL) polypeptide chains are compared by alignment below, which shows these antibodies to be distinct from one another (CDRs as determined by Kabat underlined).

| | |
|---|---|
| | CDR1 CDR2 |
| 7F9-Heavy | QMQLVQSGPEVKKPGTSVKVSCKASGFTFTSSAVQWVRQARGQRLEWIGWIVVGSGNTNY 60 |
| 9E4-heavy | QVQLVESGGGLVKPGGSLRLSCAASGFTFSDYYMSWLRQAPGRGLEWVSYISSSGITIYY 60 |
| 2A1-Heavy | EVQLVESGGGLVQPGGSLRLSCAASGFTFSSYWMSWVRQAPGKGLEWVANIKQDGSEKYY 60 |
| 11C9-heavy | QVQLVESGGGVVQPGRSLRLSCAASGFTFSSYGMHWVRQAPGKGLEWVAVIWYDGGNKDY 60 |
| 12A6-Heavy | QVQLVESGGGVVQPGRSLRLSCAASGFTFSSYGMHNVRQAPGKGLEWVAVIWYDGSDDYY 60 |
| | ::***:** : :** *:::** ****.:.. : *:*** *: ***:. : . * |
| | CDR3 |
| 7F9-Heavy | AQKFQERVTITRDMSTSTAYMELSSLRSEDTAVYYCAAGYYGSGSPYYYYYGMDVWGQGT 120 |
| 954-heavy | ADSVKGRFTISRDNAKNSLYLQMNSLRAEDTAVYYCARDG--QQLADYYYYGMDVWGQGT 118 |
| 2A1-Heavy | VDSVKGRFTISRDNAKNSLYLQMNSLRAEDTAVYYCAREV--GP--G------DYWGQGT 110 |
| 1109-heavy | ADSVKGRFTISRDNSKNTLYLQMNSLRAEDTAVYYCARVG--QL--AHYYYGMDVWGQGT 116 |
| 12A6-Heavy | ADSVKGRFTISRDNSKNTLYLQMNSIRAEDTAVYYCAREL--VR--G---ASFDYWGQGT 113 |
| | : .: *.*:: * :..: *:::.***:*********: * ***** |
| 7F9-Heavy | TVTVSS 126 |
| 954-heavy | TVTVSS 124 |
| 2A1-Heavy | LVTVSS 116 |
| 1109-heavy | TVTVSS 122 |
| 12A6-Heavy | LVTVSS 119 |
| | ***** |
| | CDR1 CDR2 |
| 7F9-light | DIQMTQSPSSVSASVGDRVTITCRASQGIS-----SWLAWYQQKSGKAPKLLIYAASSLQ 55 |
| 2A1-light | DIQMTQSPSTLSASVGDRVTITCRASQSIS-----NWVAWYQQKPGKAPKLLIHTASSLE 55 |
| 9E4-light | EIVLTQSPGTLSLSPGERATLSCRASQSVS----SSYLTWYQQKPGQAPRLLIYGASSRA 56 |
| 1109-light | DIVMTQSPLSLEVTPGEPASISCRSSQSLLHSNGYNYLDWYLQKPGQSPQLLIYLGSNRA 60 |
| 12A6-light | EILMTQSPLSIPVTPGEPATISCRSSQSLLHSNGYNYLDWYLQKPGQSPQLLIYLGSNRA 60 |
| | :* :**** :: :*: .:::*::**.: : ** ** *::*:***: .* |
| | CDR3 |
| 7F9-light | SGVPSRESGSGSGTDFTLTINSLQPEDFATYYCQQVDSFPFTFGGGTKVESK 107 |
| 2A1-Light | SGVPSRESGSGSGTEFTLTISSLQPDDFATYYCQQYYSYSPTFGQGTKVEIK 107 |
| 9E4-light | TGIPDRESGSGSGTDFTLTISRLEPEDFAVYYCQQY-GSSLTFGGGTKVEIK 107 |
| 1109-light | SGVPDRESGSGSGTDFTLKISRVEAEDVGVYYCMQALQSPPTFGQGTKVEIK 112 |
| 12A6-light | SGVPDRESGSGSGTDFTLRISRVEAEDVGVYYCMQALQTPLTFGGGTKVEIK 112 |
| | :*:*.***** ***:*** *. :: :*...*** * *** ***** * |

The alignments shown below also show these antibodies CDRs sequences for VH and VL to be distinct from Trastuzumab and Pertuzumab.

| | |
|---|---|
| | CDR1 CDR2 |
| Trastuzumab-Heavy | EVQLVESGGGLVQPGGSLRISCAASGFNIKDTYIHWVRQAPGKGLEWVARTYPTNGYTRY 60 |
| Pertuzumab-Heavy | EVQLVESGGGLVQPGGSLRLSCAASGFTFTDYTMDWVRQAPGKGLEWVADVNPNSGGSIY 60 |
| 9E4-heavy | QVQLVESGGGLVKPGGSLRISCAASGFTESDYYMSWLRQAPGRGLEWYSYISSSGITIYY 60 |
| 2A1-Heavy | EVQLVESGGGLVQPGGSIRISCAASGFTESSYWMSWVRQAPGKGLEWVANIKQDGSEKYY 60 |
| 11C9-heavy | QVQLVESGGGVVQPGRSLRLSCAASGFTFSSYGMHWVRQAPGKGLEWVAVIWYDGGNKDY 60 |
| 12A6-Heavy | QVQLVESGGGVVQPGRSIRLSCAASGFTESSYGMHWVRQAPGKGLEWVAVIWYDGSDDYY 60 |
| 7F9-Heavy | QNQLVQSGEEVKKPGTSVKVSCKASGFTFTSSAVQWVRQARGQRLEWIGWIVVGSGNTNY 60 |
| | ::***:** : :** *:::** ****.:.. : *:*** *: ***:. : . * |
| | CDR3 |
| Trastuzumab-Heavy | ADSVKGRFTISADTSKNTAYLQMNSLRAEDTAVYYCSRWG--GD----GFYAMDYWGQGT 114 |
| Pertuzumab-Heavy | NQRFKGRFTLSVDRSKNTLYLQMNSLRAEDTAVYYCARNL--GPS-----FYFDYWGQGT 113 |
| 9E4-heavy | ADSVKGRETISRDNAKNSLYLQMNSLRAEDTAVYYCARDG--QQLADYYYYGMDVWGQGT 118 |
| 2A1-Heavy | VDSVKGRETISRDNAKNSLYLQMNSLRAEDTAVYYCAREV--GP--G------DYWGQGT 110 |
| 11C9-heavy | ADSVKGRETISRDNSKNTLYLQMNSLRAEDTAVYYCARVG--QL--AHYYYGMDVWGQGT 116 |
| 12A6-Heavy | ADSVKGRFTISRDNSKNTLYLQMNSLRAEDTAVYYCAREL--VR--G---ASFDYWGQGT 113 |
| 7F9-Heavy | AQKFQERVTITRDMSTSTAYMELSSLRSEDTAVYYCAAGYYGSGSPYYYYYGMDVWGQGT 120 |
| | : .: *.*:: * :..: *:::.***:********: * ***** |
| Trastuzumab-Heavy | LVTVSS 120 |
| Pertuzumab-Heavy | LVTVSS 119 |
| 9E4-heavy | TVTVSS 124 |
| 2A1-Heavy | LVTVSS 116 |
| 1109-heavy | TVTVSS 122 |
| 12A6-Heavy | LVTVSS 119 |
| 7F9-Heavy | TVTVSS 126 |
| | CDR1 CDR2 |
| Trastuzumab-light | DIQMTQSPSSLSASVGDRVTITCRASQDVN-----TAVAWYQQKPGKAPKLLIYSASFLY 107 |
| Pertuzumab-light | DIQMTQSPSSLSASVGDRVTITCKASQDVS-----IGVAWYQQKPGKAPKLLIYSASYRY 107 |
| 9E4-light | EIVLTQSPGTLSLSPGERATLSCRASQSVS----SSYLIWYQQKPGQAPRLLIYGASSRA 107 |
| 2A1-light | DIQMTQSPSTLSASVGDRVTITCRASQSIS-----NWVAWYQQKPGKAPKLLIHTASSLE 107 |
| 1109-light | DIVMTQSPLSLPVTPGEPASISCRSSQSLLHSNGYNYLDWYLQKPGQSPQLLIYLGSNRA 107 |
| 12A6-light | EILMTQSPISIPVTPGEPATISCRSSQSLLHSNGYNYLDWYLQKPGQSPQLLIYLGSNRA 107 |
| 7F9-light | DIQMTQSPSSVSASVGDRVTITCRASQGIS-----SWLAWYQQKSGKAPKLLIYAASSLQ 107 |
| | :. :**** :: : *: .:::*::**.: ** ** *::*:***: .* |
| | CDR3 |
| Trastuzumab-light | SGVPSRESGSRSGTDETLTISSLQPEDFATYYCQQHYTTPPTEGQGTKVEIK 107 |
| Pertuzumab-light | TGVPSRESGSGSGTDETLTISSLQPEDFATYYCQQYYTYPYTEGQGTKVEIK 107 |
| 9E4-light | TGIPDRESGSGSGTDETLTISRLEPEDFAVYYCQQY-GSSLTFGGGTKVEIK 107 |
| 2A1-light | SGVPSRESGSGSGTEFTLTISSLQPDDEATYYCQQYYSYSPTEGQGTKVEIK 107 |
| 1109-light | SGVPDRESGSGSGTDETLKISRVEAEDVGVYYCMQALQSPPTEGQGTKVEIK 112 |
| 12A6-light | SGVPDRESGSGSGTDETLRISRVEAEDVGVYYCMQALQTPLTEGGGTKVEIK 112 |
| 7F9-light | SGVPSRESGSGSGTDETLTINSLQPEDFATYYCQQVDSFPETEGGGTKVESK 107 |
| | :********* **** *** *. : : :*.. , *** * *** ***** * |

Other embodiments will be apparent to those skilled in the art from consideration of the specification and directions provided herein. It is intended that the specification and examples be considered as exemplary only, with a true scope and spirit herein being indicated by the following claims.

What is claimed is:

1. An isolated antibody or antigen binding fragment thereof, comprising
a) a heavy chain variable region comprising the CDR sequences SEQ ID NOs: 1, 2, and 3 and a light chain variable region comprising CDR sequences SEQ ID NOs: 4, 5, and 6 (antibody 11D7);
b) a heavy chain variable region comprising the CDR sequences SEQ ID NOs: 1, 7 and 3 and a light chain variable region comprising CDR sequences SEQ ID NOs: 4, 5, and 6 (antibody 2A2);
c) a heavy chain variable region comprising the CDR sequences SEQ ID NOs: 1, 9 and 3 and a light chain variable region comprising CDR sequences SEQ ID NOs: 4, 5, and 6 (antibody 12E3);
d) a heavy chain variable region comprising the CDR sequences SEQ ID NOs: 1, 10 and 3 and a light chain variable region comprising CDR sequences SEQ ID NOs: 4, 5, and 6 (antibody 2D7);
e) a heavy chain variable region comprising the CDR sequences SEQ ID NOs: 1, 12, and 3 and a light chain variable region comprising CDR sequences SEQ ID NOs: 4, 5, and 6 (antibody 3G7);
f) a heavy chain variable region comprising the CDR sequences SEQ ID NOs: 1, 13, and 3 and a light chain variable region comprising CDR sequences SEQ ID NOs: 4, 5, and 6 (antibody 8C1);
g) a heavy chain variable region comprising the CDR sequences SEQ ID NOs: 14, 15 and 16 and a light chain variable region comprising CDR sequences SEQ ID NOs: 17, 18, and 19 (antibody 2A1);
h) a heavy chain variable region comprising the CDR sequences SEQ ID NOs: 20, 21 and 22 and a light chain variable region comprising CDR sequences SEQ ID NOs: 23, 24, and 25 (antibody 7F9);
i) a heavy chain variable region comprising the CDR sequences SEQ ID NOs: 26, 27, and 28 and a light chain variable region comprising CDR sequences SEQ ID NOs: 29, 30, and 31 (antibody 9E4);
j) a heavy chain variable region comprising the CDR sequences SEQ ID NOs: 32, 33, 34 and a light chain variable region comprising CDR sequences SEQ ID NOs: 35, 36 and 37 (antibody 11C9);
k) a heavy chain variable region comprising the CDR sequences SEQ ID NOs: 32, 38, and 39 and a light chain variable region comprising CDR sequences SEQ ID NOs: 35, 36 and 40 (antibody 12A6);
l) a heavy chain variable region comprising SEQ ID NO:41 and a light chain variable region comprising SEQ ID NOs: 42 (antibody 11D7);
m) a heavy chain variable region comprising SEQ ID NO:43 and a light chain variable region comprising SEQ ID NOs: 44 (antibody 2A2);
n) a heavy chain variable region comprising SEQ ID NO:45 and a light chain variable region comprising SEQ ID NOs: 46 (antibody 12E3);
o) a heavy chain variable region comprising SEQ ID NO:47 and a light chain variable region comprising SEQ ID NOs: 48 (antibody 2D7);
p) a heavy chain variable region comprising SEQ ID NO:49 and a light chain variable region comprising SEQ ID NOs: 50 (antibody 3G7);
q) a heavy chain variable region comprising SEQ ID NO:51 and a light chain variable region comprising SEQ ID NOs: 52 (antibody 8C1);
r) a heavy chain variable region comprising SEQ ID NO:53 and a light chain variable region comprising SEQ ID NOs: 54 (antibody 2A1);
s) a heavy chain variable region comprising SEQ ID NO:55 and a light chain variable region comprising SEQ ID NOs: 56 (antibody 7F9);
t) a heavy chain variable region comprising SEQ ID NO:57 and a light chain variable region comprising SEQ ID NOs: 58 (antibody 9E4);
u) a heavy chain variable region comprising SEQ ID NO:59 and a light chain variable region comprising SEQ ID NOs: 60 (antibody 11C9);
v) a heavy chain variable region comprising SEQ ID NO:61 and a light chain variable region comprising SEQ ID NOs: 62 (antibody 12A6);
w) a heavy chain variable region and corresponding a light chain variable region encoded by any of polynucleotides of SEQ ID NOS. 63-84;
and/or a conservatively substituted derivative thereof optionally comprising up to three amino acid substitutions in one or more CDRs thereof and/or up to ten amino acid substitutions of a heavy and/or light chain thereof;
wherein the antibody or antigen binding fragment thereof specifically binds to human HER2.

2. An antibody of any prior claim wherein the antibody is internalized into a cell that expresses HER2 in vitro and/or in vivo.

3. An antibody of any preceding claim that competes with trastuzumab for binding to HER2 receptor on the cell.

4. The antibody of claim 3 selected from the group consisting of antibodies 11D7, 2A2, 12E3, 2D7, 3G7, and 8C1.

5. An antibody of any preceding claim that does not compete with trastuzumab for binding to HER2 receptor on the cell.

6. The antibody of claim 5 selected from the group consisting of antibodies 2A1, 7F9, 9E4, 11C9, and 12A6.

7. The antibody of any preceding claim that is an isolated monoclonal antibody.

8. The antibody of claim 8 wherein the monoclonal antibody is a human monoclonal antibody.

9. The antibody of any preceding claim wherein said antibody is derived from a human antibody, human IgG, human IgG1, human IgG2, human IgG2a, human IgG2b, human IgG3, human IgG4, human IgM, human IgA, human IgA1, human IgA2, human IgD, human IgE, canine antibody, canine IgGA, canine IgGB, canine IgGC, canine IgGD, chicken antibody, chicken IgA, chicken IgD, chicken IgE, chicken IgG, chicken IgM, chicken IgY, goat antibody, goat IgG, mouse antibody, mouse IgG, pig antibody, rat antibody, llaman antibody, alpacan antibody, shark antibody and a camel antibody.

10. A derivative of an antibody of any preceding claim, optionally selected from the group consisting of an F_{ab}, F_{ab2}, Fab′ single chain antibody, Fv, single chain, mono-specific antibody, bispecific antibody, trimeric antibody, multi-specific antibody, multivalent antibody, chimeric antibody, canine-human chimeric antibody, canine-mouse chimeric antibody, antibody comprising a canine Fc, humanized antibody, human antibody, caninized antibody, CDR-grafted antibody, shark antibody, and a nanobody.

11. A derivative of an antibody of any preceding claim comprising a detectable label fixably attached thereto, optionally wherein the detectable label is selected from the group consisting of fluorescein, DyLight, Cy3, Cy5, FITC, HiLyte Fluor 555, HiLyte Fluor 647, 5-carboxy-2,7-dichlorofluorescein, 5-carboxyfluorescein, 5-FAM, hydroxy tryptamine, 5-hydroxy tryptamine (5-HAT), 6-carboxyfluorescein (6-FAM), FITC, 6-carboxy-1,4-dichloro-2′,7′-dichlorofluorescein (TET), 6-carboxy-1,4-dichloro-2′,4′,5′,7′-tetrachlorofluorescein (HEX), 6-carboxy-4′,5′-dichloro-2′,7′-dimethoxyfluorescein (6-JOE), an Alexa fluor, Alexa fluor 350, Alexa fluor 405, Alexa fluor 430, Alexa fluor 488, Alexa fluor 500, Alexa fluor 514, Alexa fluor 532, Alexa fluor 546, Alexa fluor 555, Alexa fluor 568, Alexa fluor 594, Alexa fluor 610, Alexa fluor 633, Alexa fluor 635, Alexa fluor 647, Alexa fluor 660, Alexa fluor 680, Alexa fluor 700, Alexa fluor 750, a BODIPY fluorophores, BODIPY 492/515, BODIPY 493/503, BODIPY 500/510, BODIPY 505/515, BODIPY 530/550, BODIPY 542/563, BODIPY 558/568, BODIPY 564/570, BODIPY 576/589, BODIPY 581/591, BODIPY 630/650-X, BODIPY 650/665-X, BODIPY 665/676, FL, FL ATP, FI-Ceramide, R6G SE, TMR, TMR-X conjugate, TMR-X, SE, TR, TR ATP, TR-X SE, a rhodamine, rhodamine 110, rhodamine 123, rhodamine B, rhodamine B 200, rhodamine BB, rhodamine BG, rhodamine B extra, 5-carboxytetramethylrhodamine (5-TAMRA), 5 GLD, 6-carboxyrhodamine 6G, Lissamine, Lissamine Rhodamine B, Phallicidine, Phalloidine, rhodamine red, Rhod-2, 6-carboxy-X-rhodamine (ROX), carboxy-X-rhodamine (5-ROX), Sulphorhodamine B can C, Sulphorhodamine G Extra, 6-carboxytetramethylrhodamine (TAMRA), tetramethylrhodamine (TRITC), rhodamine WT, Texas Red, and Texas Red-X.

12. An antibody of any of any preceding claim comprising an effector moiety attached thereto, optionally wherein the effector moiety is selected from the group consisting of a cytotoxic drug, mertansine (DM1), toxin, diphtheria A chain, exotoxin A chain, ricin A chain, abrin A chain, curcin, crotin, phenomycin, enomycin, and radiochemical.

13. The antibody of claim 12 further comprising a cleavable linker positioned between the antibody and the effector moiety, wherein said cleavable linker releases the effector moiety into or within a cell.

14. An isolated polynucleotide encoding antibody of any preceding claim, optionally wherein a nucleic acid sequence of at least one of SEQ ID NOS. 63-84.

15. An expression vector comprising one or more polynucleotides of claim 14.

16. A host cell comprising the isolated polynucleotide of claim 14 and/or the expression vector of claim 15.

17. A composition comprising at least one antibody or derivative of any one of claims 1-13; at least one isolated polynucleotide of claim 14; at least one expression vector of claim 15; and/or, at least one host cell of claim 16; or a combination thereof; and, a pharmaceutically acceptable carrier.

18. The composition of claim 17 comprising at least one first antibody or derivative of any one of claims 1-14 and trastuzumab.

19. The composition of claim 17 comprising at least two or more antibodies and/or derivatives of any one of claims 1-14.

20. The composition of claim 18 wherein the at least one first antibody is internalized into a cell that expresses HER2 in vitro and/or in vivo and/or does not compete with trastuzumab for binding to HER2.

21. The composition of claim 19 or 20, the composition further comprising trastuzumab.

22. The composition of any one of claims 17-21 comprising at least one antibody selected from the group consisting of 11D7, 2A2, 12E3, 2D7, 3G7, and 8C1 and at least one antibody selected from the group consisting of 2A1, 7F9, 9E4, 11C9, and 12A6.

23. A method for detecting HER2 on a cell, the method comprising contacting a test biological sample with an antibody or derivative of any one of claims 1-13 and detecting the antibody bound to the biological sample or components thereof.

24. The method of claim 23, further comprising comparing the amount of binding to the test biological sample or components thereof to the amount of binding to a control biological sample or components thereof, wherein increased binding to the test biological sample or components thereof relative to the control biological sample or components thereof indicates the presence of a cell expressing HER2 in the test biological sample.

25. The method of claim 23 or 24 wherein the test biological sample is a mammalian cell, tissue, or blood.

26. The method of any one of claims 23-25 wherein the method is an in vivo method or an in vitro method.

27. A method for treating, preventing and/or ameliorating cancer in a mammal comprising administering to the mammal at least one effective dose of a pharmaceutical composition comprising at least one antibody or derivative of any one of claims 1-13.

28. The method of claim 28 wherein the antibody comprises a cytotoxic effector moiety attached thereto, optionally wherein the effector moiety is selected from the group consisting of a cytotoxic drug, mertansine (DM1), toxin, diphtheria A chain, exotoxin A chain, ricin A chain, abrin A chain, curcin, crotin, phenomycin, enomycin, and radiochemical.

29. The method of claim 29 further comprising a cleavable linker positioned between the antibody and the effector moiety, wherein said cleavable linker releases the effector moiety into or within a cell.

30. The method of any one of claims 27-29 wherein the antibody is administered as an antibody-drug conjugate.

31. The method of claim 27 comprising administering at least one first antibody or derivative of any one of claims 1-13 and trastuzumab.

32. The method of claim 27 comprising administering at least two or more types of antibodies and/or derivatives of any one of claims 1-13.

33. The method of claim 27 comprising administering at least two types of antibodies, wherein at least one type of antibody is internalized into a cell that expresses HER2 in vitro and/or in vivo and/or does not compete with trastuzumab for binding to HER2.

34. The method of claim 32 wherein at least one type antibody is internalized into a cell that expresses HER2 in vitro and/or in vivo and/or at least one type of antibody competes with trastuzumab for binding to HER2 receptor on the cell.

35. The method of any one of claims 32-34, comprising administering at least one antibody selected from the group consisting of 11D7, 2A2, 12E3, 2D7, 3G7, and 8C1 and at least one antibody selected from the group consisting of 2A1, 7F9, 9E4, 11C9, and 12A6; the method optionally further comprising administering trastuzumab.

36. The method of any one of claims 27-35 wherein multiple doses are administered to the animal; and/or, the antibody is administered in a dosage amount of about 1 to 50 mg/kg.

37. A kit for detecting the expression of HER2 in or on a cell, the kit comprising at least one antibody or derivative of any preceding claim and instructions for use.

38. The kit of claim 37 wherein the antibody or derivative is in lyophilized form.

39. The kit of claim 37 or 38 wherein the at least one antibody is selected from the group consisting of 11D7, 2A2, 12E3, 2D7, 3G7, 8C1, 2A1, 7F9, 9E4, 11C9, and 12A6.

40. The kit of claim 37 or 38 wherein the at least one antibody is selected from the group consisting of 11D7, 2A2, 12E3, 2D7, 3G7, and 8C1; the kit further comprising at least one antibody selected from the group consisting of 2A1, 7F9, 9E4, 11C9, and 12A6.


SEQUENCE LISTING (206 sequences)

SEQ ID NO: 1 - protein, 5 aa, synthetic construct
DYAMH

SEQ ID NO: 2 - protein, 17 aa, synthetic construct
GIAWNGGILGYADSVKG

SEQ ID NO: 3 - protein, 12 aa, synthetic construct
DTGSYSHWYFDL

SEQ ID NO: 4 - protein, 11 aa, synthetic construct
RASQSVRSNLA

SEQ ID NO: 5 - protein, 7 aa, synthetic construct
GASTRAT

SEQ ID NO: 6 - protein, 8 aa, synthetic construct
QQYNNWPT

SEQ ID NO: 7 - protein, 17 aa, synthetic construct
GISWNGGTLGYADSVKG

SEQ ID NO: 8 - skipped

SEQ ID NO: 9 - protein, 17 aa, synthetic construct
GISWNGGTLGYAGSVKG

SEQ ID NO: 10 - protein, 17 aa, synthetic construct
GISWNSGTMGYAGSVKG

SEQ ID NO: 11 - protein, 11 aa, synthetic construct
RASQSVRTNLA

SEQ ID NO: 12 - protein, 17 aa, synthetic construct
GVSWNGGTMGYAGSVKG

SEQ ID NO: 13 - protein, 17 aa, synthetic construct
GISWNGGTLGYADSVKG

SEQ ID NO: 14 - protein, 5 aa, synthetic construct
SYWMS

SEQ ID NO: 15 - protein, 17 aa, synthetic construct
NIKQDGSEKYYVDSVKG

SEQ ID NO: 16 - protein, 7 aa, synthetic construct
EVGPGDY

SEQ ID NO: 17 - protein, 11 aa, synthetic construct
RASQSISNWVA

SEQ ID NO: 18 - protein, 7 aa, synthetic construct
TASSLES

SEQ ID NO: 19 - protein, 9 aa, synthetic construct
QQYYSYSPT

SEQ ID NO: 20 - protein, 5 aa, synthetic construct
SSAVQ

SEQ ID NO: 21 - protein, 17 aa, synthetic construct
WIVVGSGNTNYAQKFQE

SEQ ID NO: 22 - protein, 17 aa, synthetic construct
GYYGSGSPYYYYYGMDV

SEQ ID NO: 23 - protein, 11 aa, synthetic construct
RASQGISSWLA

SEQ ID NO: 24 - protein, 7 aa, synthetic construct
AASSLQS

SEQ ID NO: 25 - protein, 9 aa, synthetic construct
QQVDSFPFT

SEQ ID NO: 26 - protein, 5 aa, synthetic construct
DYYMS

SEQ ID NO: 27 - protein, 17 aa, synthetic construct
YISSSGITIYYADSVKG

SEQ ID NO: 28 - protein, 15 aa, synthetic construct
DGQQLADYYYYGMDV

SEQ ID NO: 29 - protein, 12 aa, synthetic construct
RASQSVSSSYLT

SEQ ID NO: 30 - protein, 7 aa, synthetic construct
GASSRAT

SEQ ID NO: 31 - protein, 8 aa, synthetic construct
QQYGSSLT

SEQ ID NO: 32 - protein, 5 aa, synthetic construct
SYGMH

SEQ ID NO: 33 - protein, 17 aa, synthetic construct
VIWYDGGNKDYADSVKG

SEQ ID NO: 34 - protein, 13 aa, synthetic construct
VGQLAHYYYGMDV

SEQ ID NO: 35 - protein, 16 aa, synthetic construct
RSSQSLLHSNGYNYLD

SEQ ID NO: 36 - protein, 7 aa, synthetic construct
LGSNRAS

SEQ ID NO: 37 - protein, 9 aa, synthetic construct
MQALQSPPT

SEQ ID NO: 38 - protein, 17 aa, synthetic construct
VIWYDGSDDYYADSVKG

SEQ ID NO: 39 - protein, 10 aa, synthetic construct
ELVRGASFDY

SEQ ID NO: 40 - protein, 9 aa, synthetic construct
MQALQTPLT

SEQ ID NO: 41 - protein, 121 aa, synthetic construct
EVQLVESGGGLVQPGRSLRLSCAASGFTFDDYAMHWVRQAPGKGLEWVSGIAWNGGILGYADSVKGRFTISRDNAKNSLYLQMNSLRAEDTALYYCAKDTGSYSHWYFDLWGRGTLVTVSS

SEQ ID NO: 42 - protein, 106 aa, synthetic construct
EIVMTQSPATLSVSPGERATLSCRASQSVRSNLAWYQQKPGQAPRLLIYGASTRATGIPARFSGSGSGTEFTLTISSLQSEDFAVYCCQQYNNWPTFGQGTKVEIK

SEQ ID NO: 43 - protein, 121 aa, synthetic construct
EVQLVESGGGLVQPGRSLRLSCAASGFTFDDYAMHWVRQAPGKGLEWVSGISWNGGTLGYADSVKGRFTISRDNAKNSLYLQMNSLRAEDTALYYCAKDTGSYSHWYFDLWGRGTLVTVSS

SEQ ID NO: 44 - protein, 106 aa, synthetic construct
EIVMTQSPATLSVSPGERATLSCRASQSVRSNLAWYQQKPGQAPRLLIYGASTRATGIPARFSGSGSGTEFTLTISSLHSEDFAVYYCQQYNNWPTFGQGTKVEIK

SEQ ID NO: 45 - protein, 121 aa, synthetic construct
EVQLVGSGGGLVQPGRSLRLSCAASGFTFDDYAMHWVRQAPGKGLEWVSGISWNGGTLGYAGSVKGRFTISRDNAKNSLYLQMNSLRAEDTALYYCAKDTGSYSHWYFDLWGRGTLVTVSS

SEQ ID NO: 46 - protein, 106 aa, synthetic construct
EIVMTQSPATLSVSPGERATLSCRASQSVRSNLAWYQQKPGQAPRLLIYGASTRATGIPARCSGSGSGTEFTLTISSLQSEDFAVYYCQQYNNWPTFGQGTKVEIK

SEQ ID NO: 47 - protein, 121 aa, synthetic construct
EVQLVESGGGLVQPGRSLRLSCAASGFTFDDYAMHWVRQAPGKGLEWVSGISWNSGTMGYAGSVKGRFTISRDNAKNSLYLQMNSLRAEDTALYYCAKDTGSYSHWYFDLWGRGTLVTVSS

SEQ ID NO: 48 - protein, 106 aa, synthetic construct
EIVMTQSPATLSVSPGERATLSCRASQSVRTNLAWYQQKPGQAPRLLIYGASTRATGIPARFSGSGSGTEFTLTISSLQSEDFAVYYCQQYNNWPTFGQGTKVEIK

SEQ ID NO: 49 - protein, 121 aa, synthetic construct
EVQLVESGGGLVQPGRSLRLSCAASGFTFDDYAMHWVRQAPGKGLEWVSGVSWNGGTMGYAGSVKGRFTISRDNAKNSLYLQMNSLRAEDTALYYCAKDTGSYSHWYFDLWGRGTLVTVSS

SEQ ID NO: 50 - protein, 106 aa, synthetic construct
EIVMTQSPATLSVSPGERATLSCRASQSVRSNLAWYQQKPGQAPRLLIYGASTRATGIPGRFSGSGSGTEFILTISSLQSEDFAVYYCQQYNNWPTFGQGTKVEIK

SEQ ID NO: 51 - protein, 121 aa, synthetic construct
EVQLVESGGGLVQPGRSLRLSCTASGFTFDDYAMHWVRQAPGKGLEWVSGISWNGGTLGYADSVKGRFTISRDNAKNSLYLQMNSLRAEDTALYYCAKDTGSYSHWYFDLWGRGTLVTVSS

SEQ ID NO: 52 - protein, 106 aa, synthetic construct; identical to SEQ ID NO: 44

SEQ ID NO: 53 - protein, 116 aa, synthetic construct
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCAREVGPGDYWGQGTLVTVSS

SEQ ID NO: 54 - protein, 107 aa, synthetic construct
DIQMTQSPSTLSASVGDRVTITCRASQSISNWVAWYQQKPGKAPKLLIHTASSLESGVPSRFSGSGSGTEFTLTISSLQPDDFATYYCQQYYSYSPTFGQGTKVEIK

SEQ ID NO: 55 - protein, 126 aa, synthetic construct
QMQLVQSGPEVKKPGTSVKVSCKASGFTFTSSAVQWVRQARGQRLEWIGWIVVGSGNTNYAQKFQERVTITRDMSTSTAYMELSSLRSEDTAVYYCAAGYYGSGSPYYYYYGMDVWGQGTTVTVSS

SEQ ID NO: 56 - protein, 107 aa, synthetic construct
DIQMTQSPSSVSASVGDRVTITCRASQGISSWLAWYQQKSGKAPKLLIYAASSLQSGVPSRFSGSGSGTDFTLTINSLQPEDFATYYCQQVDSFPFTFGGGTKVESK

SEQ ID NO: 57 - protein, 124 aa, synthetic construct
QVQLVESGGGLVKPGGSLRLSCAASGFTFSDYYMSWLRQAPGRGLEWVSYISSSGITIYYADSVKGRFTISRDNAKNSLYLQMNSLRAEDTAVYYCARDGQQLADYYYYGMDVWGQGTTVTVSS

SEQ ID NO: 58 - protein, 107 aa, synthetic construct
EIVLTQSPGTLSLSPGERATLSCRASQSVSSSYLTWYQQKPGQAPRLLIYGASSRATGIPDRFSGSGSGTDFTLTISRLEPEDFAVYYCQQYGSSLTFGGGTKVEIK

SEQ ID NO: 59 - protein, 122 aa, synthetic construct
QVQLVESGGGVVQPGRSLRLSCAASGFTFSSYGMHWVRQAPGKGLEWVAVIWYDGGNKDYADSVKGRFTISRDNSKNTLYLQMNSLRAEDTAVYYCARVGQLAHYYYGMDVWGQGTTVTVSS

SEQ ID NO: 60 - protein, 112 aa, synthetic construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQSPPTFGQGTKVEIK

SEQ ID NO: 61 - protein, 119 aa, synthetic construct
QVQLVESGGGVVQPGRSLRLSCAASGFTFSSYGMHWVRQAPGKGLEWVAVIWYDGSDDYYADSVKGRFTISRDNSKNTLYLQMNSLRAEDTAVYYCARELVRGASFDYWGQGTLVTVSS

SEQ ID NO: 62 - protein, 112 aa, synthetic construct
EILMTQSPLSLPVTPGEPATISCRSSQSLLHSNGYNYLDWYLQKPGQSPQLLIYLGSNRASGVPDRFSGSGSGTDFTLRISRVEAEDVGVYYCMQALQTPLTFGGGTKVEIK

SEQ ID NO: 63 - DNA, 364 bp, synthetic construct
  source 1..364: mol_type = other DNA
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattgcttggaatggtggtatcttaggctatgcggactctgtgaagggccgattcaccatctccagagacaacgccaagaactccctgtatctgcaaatgaacagtctgagagctgaggacacggccttgtattactgtgcaaaagatacggggagttattctcactggtacttcgatctctggggccgtggcaccctggtcactgtctcctcag

SEQ ID NO: 64 - DNA, 319 bp, synthetic construct
  source 1..319: mol_type = other DNA
gaaatagtgatgacgcagtctccagccaccctgtctgtgtctccaggggaaagagccaccctctcctgcagggccagtcagagtgttaggagcaacttagcctggtatcagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattgctgtcagcagtataataactggcctacgttcggccaagggaccaaggtggaaatcaaac

SEQ ID NO: 65 - DNA, 364 bp, synthetic construct
  source 1..364: mol_type = other DNA
gaagtgcagttggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaatggtggtaccttaggctatgcggactctgtgaagggccgattcaccatctccagagacaacgccaagaactccctgtatctgcaaatgaacagtctgagagctgaggacacggccttgtattactgtgcaaaagatacggggagttattctcactggtacttcgatctctggggccgtggcaccctggtcactgtctcctcag

SEQ ID NO: 66 - DNA, 319 bp, synthetic construct
  source 1..319: mol_type = other DNA
gaaatagtgatgacgcagtctccagccaccctgtctgtgtctccaggggaaagagccaccctctcctgcagggccagtcagagtgttaggagcaacttagcctggtatcagcagaaacctggccaggctcccaggctcctcatctatggtgcatccaccagggccactggtatcccagccaggttcagtggcagtgggtctgggacagagttcactctcaccattagcagcctgcactctgaagattttgcagtttattactgtcagcagtataacaactggcctacgttcggccaagggaccaaggtggaaatcaaac

SEQ ID NO: 67 - DNA, 364 bp, synthetic construct
  source 1..364: mol_type = other DNA
gaagtgcagctggtggggtctgggggaggtttggtacagcctggcaggtccctgagactctcctgtgcagcctctggattcacctttgatgattatgccatgcactgggtccggcaagctccagggaagggcctggagtgggtctcaggtattagttggaatggtggtacgttgggctatgcgggctctgtgaagggccgattcaccatctccagagacaacgccaagaactccctgtatctgcaaatgaacagtctgagagctgaggacacggccttgtattactgtgcaaaagatacggggagttattctcactggtacttcgatctctggggccgtggcaccctggtcactgtctcctcag

SEQ ID NO: 68 - DNA, 319 bp, synthetic construct
  source 1..319: mol_type = other DNA
gaaatagtgatgacgcagtctccagccaccctgtctgtgtctccaggggaaagagccaccctctcctgcagggccagtcagagtgttagaagcaacttagcctggtaccagcagaaacctggccaggctcccaggctcctcatctatggtgcatccaccagggccactggtatcccagccaggtgcagtggcagtgggtctgggacagagttcactctcaccatcagcagcctgcagtctgaggattttgcagtttattactgtcagcagtataataactggcctacgttcggccaagggaccaaggtggaaatcaaac

SEQ ID NO: 69 - DNA, 364 bp, synthetic construct
  source 1..364: mol_type = other DNA
gaagtgcagctggtggagtctgggggaggtttggtacagcctggcaggtccctgagactctcctgtgcagcctctggattcacctttgatgattatgccatgcactgggtccggcaagctccagggaagggcctggagtgggtctcaggtattagttggaatagtggtaccatgggctatgcgggctctgtgaagggccgattcaccatctccagagacaacgccaagaactccctgtatctgcaaatgaacagtctgagagctgaggacacggccttgtattactgtgcaaaagatacggggagttattctcactggtacttcgatctctggggccgtggcaccctggtcactgtctcctcag

SEQ ID NO: 70 - DNA, 319 bp, synthetic construct
  source 1..319: mol_type = other DNA
gaaatagtgatgacgcagtctccagccaccctgtctgtgtctccaggggaaagagccaccctctcctgcagggccagtcagagtgttaggaccaacttagcctggtaccagcagaaacctggccaggctcccaggctcctcatctatggtgcatccaccagggccactggcatcccagccaggttcagtggtagtgggtctgggacagagttcactctcaccatcagcagcctgcagtctgaagattttgcagtttattactgtcagcagtataataactggcctacgttcggccaagggaccaaggtggaaatcaaac

SEQ ID NO: 71 - DNA, 364 bp, synthetic construct
  source 1..364: mol_type = other DNA
gaagtgcagctggtggagtctgggggaggtttggtacagcctggcaggtccctgagactctcctgtgcagcctctggattcacctttgatgattatgccatgcactgggtccggcaagctccagggaagggcctggagtgggtctcaggtgttagttggaatggtggtaccatgggctatgcgggctctgtgaagggccgattcaccatctccagagacaacgccaagaactccctgtatctgcaaatgaacagtctgagagctgaggacacggccttgtattactgtgcaaaagatacggggagttattctcactggtacttcgatctctggggccgtggcaccctggtcactgtctcctcag

SEQ ID NO: 72 - DNA, 319 bp, synthetic construct
  source 1..319: mol_type = other DNA
gaaatagtgatgacgcagtctccagccaccctgtctgtgtctccaggggaaagagccaccctctcctgcagggccagtcagagtgttaggagcaacttagcctggtaccagcagaaacctggccaggctcccaggctcctcatctatggtgcatccaccagggccactggtatcccaggcaggttcagtggcagtgggtctgggacagagttcattctcaccatcagcagcctgcagtctgaagattttgcagtttattactgtcagcagtataataactggcctacgttcggccaagggaccaaggtggaaatcaaac

SEQ ID NO: 73 - DNA, 364 bp, synthetic construct
  source 1..364: mol_type = other DNA
gaagtgcagttggtggagtctgggggaggcttggtacagcctggcaggtccctgagactctcctgtacagcctctggattcacctttgatgattatgccatgcactgggtccggcaagctccagggaagggcctggagtgggtctcaggtattagttggaatggtggtaccttaggctatgcggactctgtgaagggccgattcaccatctccagagacaacgccaagaactccctgtatctgcaaatgaacagtctgagagctgaggacacggccttgtattactgtgcaaaagatacggggagttattctcactggtacttcgatctctggggccgtggcaccctggtcactgtctcctcag

SEQ ID NO: 74 - DNA, 319 bp, synthetic construct
  source 1..319: mol_type = other DNA
gaaatagtgatgacgcagtctccagccaccctgtctgtgtctccaggggaaagagccaccctctcctgtagggccagtcagagtgttaggagcaacttagcctggtatcagcagaaacctggccaggctcccaggctcctcatctatggtgcatccaccagggccactggtatcccagccaggttcagtggcagtgggtctgggacagagttcactctcaccattagcagcctgcactctgaagattttgcagtttattactgtcagcagtataacaactggcctacgttcggccaagggaccaaggtggaaatcaaac

SEQ ID NO: 75 - DNA, 349 bp, synthetic construct
  source 1..349: mol_type = other DNA
gaggtgcagctggtggagtctgggggaggcttggtccagcctggggggtccctgagactctcctgtgcagcctctggattcacctttagtagctattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtgtattactgtgcgagagaggtggggcccggggactactggggccagggaaccctggtcaccgtctcctcag

SEQ ID NO: 76 - DNA, 322 bp, synthetic construct
  source 1..322: mol_type = other DNA
gacatccagatgacccagtctccttccaccctgtctgcatctgtaggagacagagtcaccatcacttgccgggccagtcagagtattagtaactgggtggcctggtatcagcagaaaccagggaaagcccctaagctcctgatccatacggcatctagcttagaaagtggggtcccatcaaggttcagcggcagtggatctgggacagaattcactctcaccatcagcagcctgcagcctgatgattttgcaacttattactgccaacagtattatagttattctccgacgttcggccaagggaccaaggtggaaatcaaac

SEQ ID NO: 77 - DNA, 379 bp, synthetic construct
  source 1..379: mol_type = other DNA
caaatgcagctggtgcagtctgggcctgaggtgaagaagcctgggacctcagtgaaggtctcctgcaaggcttctggattcacctttactagctctgctgtgcagtgggtgcgacaggctcgtggacaacgccttgagtggataggatggatcgtcgttggcagtggtaacacaaactacgcacagaagttccaggaaagagtcaccattaccagggacatgtccacaagcacagcctacatggagctgagcagcctgagatccgaggacacggccgtgtattactgtgcggcggggtactatggttcggggagtccttactactactactacggtatggacgtctggggccaagggaccacggtcaccgtctcctcag

SEQ ID NO: 78 - DNA, 322 bp, synthetic construct
  source 1..322: mol_type = other DNA
gacatccagatgacccagtctccatcttccgtgtctgcatctgtaggagacagagtcaccatcacttgtcgggcgagtcagggtattagcagctggttagcctggtatcagcagaaatcagggaaagcccctaagctcctgatctatgctgcatctagtttgcaaagtggggtcccatcaaggttcagcggcagtggatctgggacagatttcactctcaccatcaacagcctgcagcctgaagattttgcaacttactattgtcaacaggttgacagtttcccgttcactttcggcggagggaccaaggtggagagcaaac

SEQ ID NO: 79 - DNA, 373 bp, synthetic construct
  source 1..373: mol_type = other DNA
caggtgcagctggtggagtctgggggaggcttggtcaagcctggagggtccctgagactctcctgtgcagcctctggattcaccttcagtgactactacatgagctggctccgccaggctccagggagggggctggagtgggtttcatacattagtagtagtggtattaccatatactacgcagactctgtgaagggccgattcaccatctccagggacaacgccaagaactcactgtatctgcaaatgaacagcctgagagccgaggacacggctgtgtattactgtgcgagagatggacagcagctggccgactactactactacggtatggacgtctggggccaagggaccacggtcaccgtctcctcag

SEQ ID NO: 80 - DNA, 322 bp, synthetic construct
  source 1..322: mol_type = other DNA
gaaattgtgttgacgcagtctccaggcaccctgtctttgtctccaggggaaagagccaccctctcctgcagggccagtcagagtgttagcagcagctacttaacctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcactcactttcggcggagggaccaaggtggagatcaaac

SEQ ID NO: 81 - DNA, 367 bp, synthetic construct
  source 1..367: mol_type = other DNA
caggtgcagctggtggagtctgggggaggcgtggtccagcctgggaggtccctgagactctcctgtgcagcgtctggattcacgttcagtagctatggcatgcactgggtccgccaggctccaggcaaggggctggagtgggtggcagttatatggtatgatggaggtaataaagactatgcagactccgtgaagggccgattcaccatctccagagacaattccaagaacacgctgtatctgcaaatgaacagcctgagagccgaggacacggctgtgtattactgtgcgagagtagggcagctggcacactactactacggtatggacgtctggggccaagggaccacggtcaccgtctcctcag

SEQ ID NO: 82 - DNA, 337 bp, synthetic construct
  source 1..337: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaagtcctccgacgttcggccaagggaccaaggtggaaatcaaac

SEQ ID NO: 83 - DNA, 358 bp, synthetic construct
  source 1..358: mol_type = other DNA
caggtgcaactggtggagtctgggggaggcgtggtccagcctgggaggtccctgagactctcctgtgcagcgtctggcttcaccttcagtagctatggcatgcactgggtccgccaggctccaggcaaggggctggagtgggtggcagttatatggtatgatggaagtgatgactactatgcagactccgtgaagggccgattcaccatctccagagacaattccaagaacacgctgtatctgcaaatgaacagcctgagagccgaggacacggctgtgtattactgtgcgagagagctggttcggggagcctcctttgactactggggccagggaaccctggtcaccgtctcctcag

SEQ ID NO: 84 - DNA, 337 bp, synthetic construct
  source 1..337: mol_type = other DNA
gaaattttgatgactcagtctccactctccctgcccgtcacccctggagagccggccaccatctcctgcaggtctagtcagagcctcctccatagtaatggatacaactatttggattggtacctgcagaagccagggcagtctccacagctcctgatctatttgggttctaatcgggcctccggggtccctgacaggttcagtggcagtggatcaggcacagattttacactgagaatcagcagagtggaggctgaggatgttggggtttattactgcatgcaagctctacaaactccgctcactttcggcggagggaccaaggtggagatcaaac

SEQ ID NO: 85 - protein, 30 aa, synthetic construct
EVQLVESGGGLVQPGRSLRLSCAASGFTFD

SEQ ID NO: 86 - protein, 14 aa, synthetic construct
WVRQAPGKGLEWVS

SEQ ID NO: 87 - protein, 32 aa, synthetic construct
RFTISRDNAKNSLYLQMNSLRAEDTALYYCAK

SEQ ID NO: 88 - protein, 11 aa, synthetic construct
WGRGTLVTVSS

SEQ ID NO: 89 - protein, 23 aa, synthetic construct
EIVMTQSPATLSVSPGERATLSC

SEQ ID NO: 90 - protein, 15 aa, synthetic construct
WYQQKPGQAPRLLIY

SEQ ID NO: 91 - protein, 32 aa, synthetic construct
GIPARFSGSGSGTEFTLTISSLQSEDFAVYCC

SEQ ID NO: 92 - protein, 10 aa, synthetic construct
FGQGTKVEIK

SEQ ID NO: 93 - protein, 30 aa, synthetic construct; identical to SEQ ID NO: 85

SEQ ID NO: 94 - protein, 14 aa, synthetic construct
WVRQAPGKGLEWVS

SEQ ID NO: 95 - protein, 32 aa, synthetic construct; identical to SEQ ID NO: 87

SEQ ID NO: 96 - protein, 11 aa, synthetic construct
WGRGTLVTVSS

SEQ ID NO: 97 - protein, 23 aa, synthetic construct
EIVMTQSPATLSVSPGERATLSC

SEQ ID NO: 98 - protein, 15 aa, synthetic construct
WYQQKPGQAPRLLIY

SEQ ID NO: 99 - protein, 32 aa, synthetic construct
GIPARFSGSGSGTEFTLTISSLHSEDFAVYYC

SEQ ID NO: 100 - protein, 10 aa, synthetic construct
FGQGTKVEIK

SEQ ID NO: 101 - protein, 30 aa, synthetic construct
EVQLVGSGGGLVQPGRSLRLSCAASGFTFD

SEQ ID NO: 102 - protein, 14 aa, synthetic construct
WVRQAPGKGLEWVS

SEQ ID NO: 103 - protein, 32 aa, synthetic construct; identical to SEQ ID NO: 87

SEQ ID NO: 104 - protein, 11 aa, synthetic construct
WGRGTLVTVSS

SEQ ID NO: 105 - protein, 23 aa, synthetic construct
EIVMTQSPATLSVSPGERATLSC

SEQ ID NO: 106 - protein, 15 aa, synthetic construct
WYQQKPGQAPRLLIY

SEQ ID NO: 107 - protein, 32 aa, synthetic construct
GIPARCSGSGSGTEFTLTISSLQSEDFAVYYC

SEQ ID NO: 108 - protein, 10 aa, synthetic construct
FGQGTKVEIK

SEQ ID NO: 109 - protein, 30 aa, synthetic construct; identical to SEQ ID NO: 85

SEQ ID NO: 110 - protein, 14 aa, synthetic construct
WVRQAPGKGLEWVS

SEQ ID NO: 111 - protein, 32 aa, synthetic construct; identical to SEQ ID NO: 87

SEQ ID NO: 112 - protein, 11 aa, synthetic construct
WGRGTLVTVSS

SEQ ID NO: 113 - protein, 23 aa, synthetic construct
EIVMTQSPATLSVSPGERATLSC

SEQ ID NO: 114 - protein, 15 aa, synthetic construct
WYQQKPGQAPRLLIY

SEQ ID NO: 115 - protein, 32 aa, synthetic construct
GIPARFSGSGSGTEFTLTISSLQSEDFAVYYC

SEQ ID NO: 116 - protein, 10 aa, synthetic construct
FGQGTKVEIK

SEQ ID NO: 117 - protein, 30 aa, synthetic construct; identical to SEQ ID NO: 85

SEQ ID NO: 118 - protein, 14 aa, synthetic construct
WVRQAPGKGLEWVS

SEQ ID NO: 119 - protein, 32 aa, synthetic construct; identical to SEQ ID NO: 87

SEQ ID NO: 120 - protein, 11 aa, synthetic construct
WGRGTLVTVSS

SEQ ID NO: 121 - protein, 23 aa, synthetic construct
EIVMTQSPATLSVSPGERATLSC

SEQ ID NO: 122 - protein, 15 aa, synthetic construct
WYQQKPGQAPRLLIY

SEQ ID NO: 123 - protein, 32 aa, synthetic construct
GIPGRFSGSGSGTEFILTISSLQSEDFAVYYC

SEQ ID NO: 124 - protein, 10 aa, synthetic construct
FGQGTKVEIK

SEQ ID NO: 125 - protein, 30 aa, synthetic construct
EVQLVESGGGLVQPGRSLRLSCTASGFTFD

SEQ ID NO: 126 - protein, 14 aa, synthetic construct
WVRQAPGKGLEWVS

SEQ ID NO: 127 - protein, 32 aa, synthetic construct; identical to SEQ ID NO: 87

SEQ ID NO: 128 - protein, 11 aa, synthetic construct
WGRGTLVTVSS

SEQ ID NO: 129 - protein, 23 aa, synthetic construct
EIVMTQSPATLSVSPGERATLSC

SEQ ID NO: 130 - protein, 15 aa, synthetic construct
WYQQKPGQAPRLLIY

SEQ ID NO: 131 - protein, 32 aa, synthetic construct; identical to SEQ ID NO: 99

SEQ ID NO: 132 - protein, 10 aa, synthetic construct
FGQGTKVEIK

SEQ ID NO: 133 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 214 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 448 aa, synthetic construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 137 - protein, 30 aa, synthetic construct
EVQLVESGGGLVQPGGSLRLSCAASGFTFS

SEQ ID NO: 138 - protein, 14 aa, synthetic construct
WVRQAPGKGLEWVA

SEQ ID NO: 139 - protein, 32 aa, synthetic construct
RFTISRDNAKNSLYLQMNSLRAEDTAVYYCAR

SEQ ID NO: 140 - protein, 11 aa, synthetic construct
WGQGTLVTVSS

SEQ ID NO: 141 - protein, 23 aa, synthetic construct
DIQMTQSPSTLSASVGDRVTITC

SEQ ID NO: 142 - protein, 15 aa, synthetic construct
WYQQKPGKAPKLLIH

SEQ ID NO: 143 - protein, 32 aa, synthetic construct
GVPSRFSGSGSGTEFTLTISSLQPDDFATYYC

SEQ ID NO: 144 - protein, 10 aa, synthetic construct
FGQGTKVEIK

SEQ ID NO: 145 - protein, 30 aa, synthetic construct
QMQLVQSGPEVKKPGTSVKVSCKASGFTFT

SEQ ID NO: 146 - protein, 14 aa, synthetic construct
WVRQARGQRLEWIG

SEQ ID NO: 147 - protein, 32 aa, synthetic construct
RVTITRDMSTSTAYMELSSLRSEDTAVYYCAA

SEQ ID NO: 148 - protein, 11 aa, synthetic construct
WGQGTTVTVSS

SEQ ID NO: 149 - protein, 23 aa, synthetic construct
DIQMTQSPSSVSASVGDRVTITC

SEQ ID NO: 150 - protein, 15 aa, synthetic construct
WYQQKSGKAPKLLIY

SEQ ID NO: 151 - protein, 32 aa, synthetic construct
GVPSRFSGSGSGTDFTLTINSLQPEDFATYYC

SEQ ID NO: 152 - protein, 10 aa, synthetic construct
FGGGTKVESK

SEQ ID NO: 153 - protein, 30 aa, synthetic construct
QVQLVESGGGLVKPGGSLRLSCAASGFTFS

SEQ ID NO: 154 - protein, 14 aa, synthetic construct
WLRQAPGRGLEWVS

SEQ ID NO: 155 - protein, 32 aa, synthetic construct; identical to SEQ ID NO: 139

SEQ ID NO: 156 - protein, 11 aa, synthetic construct
WGQGTTVTVSS

SEQ ID NO: 157 - protein, 23 aa, synthetic construct
EIVLTQSPGTLSLSPGERATLSC

SEQ ID NO: 158 - protein, 15 aa, synthetic construct
WYQQKPGQAPRLLIY

SEQ ID NO: 159 - protein, 32 aa, synthetic construct
GIPDRFSGSGSGTDFTLTISRLEPEDFAVYYC

SEQ ID NO: 160 - protein, 10 aa, synthetic construct
FGGGTKVEIK

SEQ ID NO: 161 - protein, 30 aa, synthetic construct
QVQLVESGGGVVQPGRSLRLSCAASGFTFS

SEQ ID NO: 162 - protein, 14 aa, synthetic construct
WVRQAPGKGLEWVA

SEQ ID NO: 163 - protein, 32 aa, synthetic construct
RFTISRDNSKNTLYLQMNSLRAEDTAVYYCAR

SEQ ID NO: 164 - protein, 11 aa, synthetic construct
WGQGTTVTVSS

SEQ ID NO: 165 - protein, 23 aa, synthetic construct
DIVMTQSPLSLPVTPGEPASISC

SEQ ID NO: 166 - protein, 15 aa, synthetic construct
WYLQKPGQSPQLLIY

SEQ ID NO: 167 - protein, 32 aa, synthetic construct
GVPDRFSGSGSGTDFTLKISRVEAEDVGVYYC

SEQ ID NO: 168 - protein, 10 aa, synthetic construct
FGQGTKVEIK

SEQ ID NO: 169 - protein, 30 aa, synthetic construct; identical to SEQ ID NO: 161

SEQ ID NO: 170 - protein, 14 aa, synthetic construct
WVRQAPGKGLEWVA

SEQ ID NO: 171 - protein, 32 aa, synthetic construct; identical to SEQ ID NO: 163

SEQ ID NO: 172 - protein, 11 aa, synthetic construct
WGQGTLVTVSS

SEQ ID NO: 173 - protein, 23 aa, synthetic construct
EILMTQSPLSLPVTPGEPATISC

SEQ ID NO: 174 - protein, 15 aa, synthetic construct
WYLQKPGQSPQLLIY

SEQ ID NO: 175 - protein, 32 aa, synthetic construct
GVPDRFSGSGSGTDFTLRISRVEAEDVGVYYC

SEQ ID NO: 176 - protein, 10 aa, synthetic construct
FGGGTKVEIK

SEQ ID NO: 177 - protein, 121 aa, synthetic construct
EVQLVGSGGGLVQPGRSLRLSCAASGFTFDDYAMHWVRQAPGKGLEWVSGISWNGGTLGYAGSVKGRFTISRDNAKNSLYLOMNSLRAEDTALYYCAKDTGSYSHWYFDLWGRGTLVTVSS

SEQ ID NO: 178 - protein, 121 aa, synthetic construct
EVOLVESGGGLVQPGRSLRLSCAASGFTFDDYAMHWVRQAPGKGLEWVSGIAWNGGILGYADSVKGRFTISRDNAKNSLYLOMNSLRAEDTALYYCAKDTGSYSHWYFDLWGRGTLVTVSS

SEQ ID NO: 179 - protein, 121 aa, synthetic construct
EVOLVESGGGLVQPGRSLRLSCTASGFTFDDYAMHWVRQAPGKGLEWVSGISWNGGTLGYADSVKGRFTISRDNAKNSLYLOMNSLRAEDTALYYCAKDTGSYSHWYFDLWGRGTLVTVSS

SEQ ID NO: 180 - protein, 121 aa, synthetic construct
EVOLVESGGGLVQPGRSLRLSCAASGFTFDDYAMHWVRQAPGKGLEWVSGISWNGGTLGYADSVKGRFTISRDNAKNSLYLQMNSLRAEDTALYYCAKDTGSYSHWYFDLWGRGTLVTVSS

SEQ ID NO: 181 - protein, 121 aa, synthetic construct
EVOLVESGGGLVQPGRSLRLSCAASGFTFDDYAMHWVRQAPGKGLEWVSGISWNSGTMGYAGSVKGRFTISRDNAKNSLYLQMNSLRAEDTALYYCAKDTGSYSHWYFDLWGRGTLVTVSS

SEQ ID NO: 182 - protein, 121 aa, synthetic construct
EVOLVESGGGLVQPGRSLRLSCAASGFTFDDYAMHWVRQAPGKGLEWVSGVSWNGGTMGYAGSVKGRFTISRDNAKNSLYLQMNSLRAEDTALYYCAKDTGSYSHWYFDLWGRGTLVTVSS

SEQ ID NO: 183 - protein, 120 aa, synthetic construct
EVOLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSS

SEQ ID NO: 184 - protein, 119 aa, synthetic construct
EVOLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSS

SEQ ID NO: 185 - protein, 107 aa, synthetic construct [sequence omitted]

SEQ ID NO: 186 - protein, 107 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQOYYIYPYTFGQGTKVEIK

SEQ ID NO: 187 - protein, 120 aa, synthetic construct
EVOLVGSGGGLVQPGRSLRLSCAASGFTFDDYAMHWVRQAPGKGLEWVSGISWNGGTLGYAGSVKGRFTISRDNAKNSLYLQMNSLRAEDTALYYCAKDTGSYSHWYFDLWGRGTLVTVS

SEQ ID NO: 188 - protein, 120 aa, synthetic construct
EVOLVESGGGLVQPGRSLRLSCAASGFTFDDYAMHWVRQAPGKGLEWVSGIAWNGGILGYADSVKGRFTISRDNAKNSLYLQMNSLRAEDTALYYCAKDTGSYSHWYFDLWGRGTLVTVS

SEQ ID NO: 189 - protein, 120 aa, synthetic construct
EVQLVESGGGLVQPGRSLRLSCTASGFTFDDYAMHWVRQAPGKGLEWVSGISWNGGTLGYADSVKGRFTISRDNAKNSLYLQMNSLRAEDTALYYCAKDTGSYSHWYFDLWGRGTLVTVS

SEQ ID NO: 190 - protein, 120 aa, synthetic construct
EVOLVESGGGLVQPGRSLRLSCAASGFTFDDYAMHWVRQAPGKGLEWVSGISWNGGTLGYADSVKGRFTISRDNAKNSLYLQMNSLRAEDTALYYCAKDTGSYSHWYFDLWGRGTLVTVS

SEQ ID NO: 191 - protein, 120 aa, synthetic construct
EVOLVESGGGLVQPGRSLRLSCAASGFTFDDYAMHWVRQAPGKGLEWVSGISWNSGTMGYAGSVKGRFTISRDNAKNSLYLQMNSLRAEDTALYYCAKDTGSYSHWYFDLWGRGTLVTVS

SEQ ID NO: 192 - protein, 120 aa, synthetic construct
EVOLVESGGGLVQPGRSLRLSCAASGFTFDDYAMHWVRQAPGKGLEWVSGVSWNGGTMGYAGSVKGRFTISRDNAKNSLYLOMNSLRAEDTALYYCAKDTGSYSHWYFDLWGRGTLVTVS

SEQ ID NO: 193 - protein, 106 aa, synthetic construct
EIVMTOSPATLSVSPGERATLSCRASQSVRSNLAWYQQKPGQAPRLLIYGASTRATGIPGRFSGSGSGTEFILTISSLOSEDFAVYYCQOYNNWPTFGOGTKVEIK

SEQ ID NO: 194 - protein, 126 aa, synthetic construct
QMQLVQSGPEVKKPGTSVKVSCKASGFTFTSSAVQWVRQARGORLEWIGWIVVGSGNTNYAQKFQERVTITRDMSTSTAYMELSSLRSEDTAVYYCAAGYYGSGSPYYYYYGMDVWGQGTTVTVSS

SEQ ID NO: 195 - protein, 116 aa, synthetic construct
EVOLVESGGGLVQPGGSLRLSCAASGFTFSSYWMSWVRQAPGKGLEWVANIKQDGSEKYYVDSVKGRFTISRDNAKNSLYLOMNSLRAEDTAVYYCAREVGPGDYWGQGTLVTVSS

SEQ ID NO: 196 - protein, 107 aa, synthetic construct
DIQMTQSPSSVSASVGDRVTITCRASQGISSWLAWYQQKSGKAPKLLIYAASSLOSGVPSRFSGSGSGTDFTLTINSLOPEDFATYYCQQVDSFPFTFGGGTKVESK

SEQ ID NO: 197 - protein, 107 aa, synthetic construct
DIQMTQSPSTLSASVGDRVTITCRASQSISNWVAWYQQKPGKAPKLLIHTASSLESGVPSRFSGSGSGTEFTLTISSLOPDDFATYYCQOYYSYSPTFGQGTKVEIK

SEQ ID NO: 198 - protein, 107 aa, synthetic construct
EIVLTQSPGTLSLSPGERATLSCRASQSVSSSYLTWYQQKPGQAPRLLIYGASSRATGIPDRFSGSGSGTDFTLTISRLEPEDFAVYYCQOYGSSLTFGGGTKVEIK

SEQ ID NO: 199 - protein, 119 aa, synthetic construct; identical to SEQ ID NO: 184

SEQ ID NO: 200 - protein, 116 aa, synthetic construct
EVOLVESGGGLVQPGGSLRLSCAASGFTFSSYWMSWVRQAPGKGLEWVANIKQDGSEKYYVDSVKGRFTISRDNAKNSLYLQMNSLRAEDTAVYYCAREVGPGDYWGQGTLVTVSS

SEQ ID NO: 201 - protein, 119 aa, synthetic construct
QVQLVESGGGVVQPGRSLRLSCAASGFTFSSYGMHWVRQAPGKGLEWVAVIWYDGSDDYYADSVKGRFTISRDNSKNTLYLOMNSLRAEDTAVYYCARELVRGASFDYWGQGTLVTVSS

SEQ ID NO: 202 - protein, 107 aa, synthetic construct [sequence omitted]

SEQ ID NO: 203 - protein, 107 aa, synthetic construct
DIQMTQSPSTLSASVGDRVTITCRASQSISNWVAWYQQKPGKAPKLLIHTASSLESGVPSRFSGSGSGTEFTLTISSLQPDDFATYYCQOYYSYSPTFGQGTKVEIK

SEQ ID NO: 204 - protein, 112 aa, synthetic construct
EILMTQSPLSLPVTPGEPATISCRSSQSLLHSNGYNYLDWYLQKPGQSPQLLIYLGSNRASGVPDRFSGSGSGTDFTLRISRVEAEDVGVYYCMOALOTPLTFGGGTKVEIK

SEQ ID NO: 205 - protein, 107 aa, synthetic construct
DIQMTQSPSSVSASVGDRVTITCRASQGISSWLAWYQQKSGKAPKLLIYAASSLQSGVPSRFSGSGSGTDFTLTINSLOPEDFATYYCQOVDSFPFTFGGGTKVESK

SEQ ID NO: 206 - DNA, 15 bp, synthetic construct
  source 1..15: mol_type = other DNA
gattatgctatgcat